(19) United States
(12) Patent Application Publication
(10) Pub. No.: US 2020/0253996 A1
(43) Pub. Date: Aug. 13, 2020

(54) TCTP INHIBITING AGENTS FOR THE TREATMENT OF PROLIFERATIVE DISEASES, INFECTIOUS DISEASES, ALLERGIES, INFLAMMATIONS AND/OR ASTHMA

(71) Applicants: UNIVERSITE PARIS-SUD, Orsay (FR); CENTRE NATIONAL DE LA RECHERCHE SCIENTIFIQUE (CNRS), Paris (FR); INSTITUT NATIONAL DE LA SANTE ET DE LA RECHERCHE MEDICALE (INSERM), Paris (FR); INSTITUT GUSTAVE-ROUSSY, Villejuif (FR)

(72) Inventors: Samir MESSAOUDI, Chilly Mazarin (FR); Mouad ALAMI, Bussy Saint Georges (FR); Jean-Daniel BRION, Saint Leu La Foret (FR); Amélie CHABRIER, Cachan (FR); Adam TELERMAN, Paris (FR); Robert AMSON, Paris (FR)

(73) Assignees: UNIVERSITE PARIS-SUD, Orsay (FR); CENTRE NATIONAL DE LA RECHERCHE SCIENTIFIQUE (CNRS), Paris (FR); INSTITUT NATIONAL DE LA SANTE ET DE LA RECHERCHE MEDICALE (INSERM), Paris (FR); INSTITUT GUSTAVE-ROUSSY, Villejuif (FR)

(21) Appl. No.: 16/651,719

(22) PCT Filed: Oct. 1, 2018
(86) PCT No.: PCT/FR2018/052410

§ 371 Date: Mar. 27, 2020

(30) Foreign Application Priority Data
Sep. 29, 2017 (FR) 1759102

(51) Int. Cl. A61K 31/706; A61K 31/7068; A61P 35/00; A61K 31/015; A61K 31/085; A61K 31/03; A61K 31/09; A61K 31/235; A61K 31/11; A61K 31/165; A61K 31/352; A61K 31/404; A61K 31/472; A61K 31/382; A61K 31/704; A61K 31/197; A61K 31/24; A61K 31/135

(52) U.S. Cl. CPC A61K 31/706; A61K 31/135; A61P 35/00; A61K 31/015; A61K 31/085; A61K 31/03; A61K 31/09; A61K 31/235; A61K 31/11; A61K 31/165; A61K 31/352; A61K 31/404; A61K 31/472; A61K 31/382; A61K 31/704; A61K 31/197; A61K 31/24; A61K 31/7068

(57) ABSTRACT

The present invention relates to the compounds of formula (I) below:

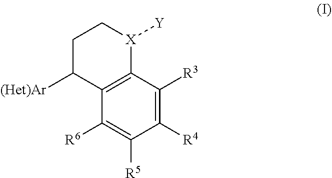

wherein:
X represents an oxygen atom, a sulfur atom, a nitrogen atom or a CH radical,
The bond X—Y and Y are absent if X represents an oxygen or sulfur atom, the bond X—Y and Y are present if X represents a nitrogen atom or a CH radical,
When present, Y represents
   a group R if X represents a nitrogen atom,
   a hydrogen atom or a group —NR¹R² if X represents a CH radical,
(Het)Ar is an aromatic ring selected from the group consisting of aryl and heteroaryl groups,
R³, R⁴, R⁵, R⁶ represent, independently of one another, a hydrogen atom, a halogen atom, a —NR¹²R¹³, a —SR¹⁴ group, a —OR¹⁴ group or a —CF₃ group,
When Y is —NR¹R², the groups —NR¹R² and (Het)Ar are in the cis-conformation,
   or a pharmaceutically acceptable salt thereof,
   for use in the treatment of proliferative diseases, infectious diseases, allergies, inflammation and/or asthma.

### FIELD OF THE INVENTION

The invention relates to TCTP inhibiting compounds for use in the treatment of proliferative diseases, infectious diseases, allergies, inflammation and/or asthma.

### PRIOR ART

Cancer is a major cause of death worldwide, with a number of new cases continuing to rise. According to the WHO, as many as 15 million deaths are due to cancer worldwide each year, or almost 1 death every two seconds. Cancer has already claimed 84 million victims between 2005 and 2015 and if nothing is done, it will kill 19 million people a year by 2025. Among the therapies used, so-called conventional antitumor chemotherapy involving cytotoxic agents, alone or in combination with surgery or radiotherapy, is the predominant approach. However, treatments are frequently accompanied by adverse effects due to a lack of selectivity towards tumor cells. In addition, multi-resistance, the main mechanism by which many cancers escape treatment, is a major factor in the failure of many chemotherapies. Consequently, the latter must constantly evolve in order to remove these main barriers. Recent advances in cancer treatment are linked to the advent of “targeted therapies” specifically targeting certain mechanisms involved in cell regulation and growth. This more rational approach has significantly changed patient management. The active agents used are generally better tolerated and do not cause the side effects specific to conventional chemotherapy (alopecia, nausea, vomiting). However, they can cause toxicities such as increased blood pressure, headaches, proteinuria, allergic reactions or digestive problems.

Targeted therapies include several families of antitumor drugs: monoclonal antibodies, tyrosine kinase receptor blockers and angiogenesis inhibitors. Despite the value of these targeted therapies, current treatments have shown limited results due to the high biological diversity of cancers and the development of resistance phenomena.

Tumor reversion (1, 3) has recently emerged as a cellular process in its own right, leading to reprogramming of the cancer cell and the disappearance of its malignant phenotype. Forcing tumor cells to “revert” to become “pseudo” normal is a wise strategy. The mechanism of tumor reversion, through the identification of proteins that play a major role in this process, is thus an extremely promising avenue for the development of new antitumor agents. In this respect, translationally controlled tumor protein (TCTP) (4) has been identified as a key element in tumor reversion. Indeed, it is overexpressed in many cancer cells and has antiapoptotic activity using p53/MDM2 and/or Bcl-xL/Mcl-1 dependent pathways. The loss of the malignant phenotype in tumor reversion is therefore likely to require the restoration of normal apoptotic activity following at least partial inactivation of the action of TCTP in the cell.

TCTP is also involved in the infection mechanisms of several parasites, including Plasmodium falciparum, which is responsible for malaria, and its inhibition makes it possible to prevent or treat infections induced by these parasites (6, 7).

In particular, TCTP can be inhibited by sertraline (WO 2004/080445). Despite sertraline's antitumor action in humans, its affinity for TCTP remains relatively low (Kd=198 μM). Therefore, the required effective dose is close to its maximum tolerated dose (MTD=400 mg/day) and leads to undesirable side effects. This phenomenon has been observed in Phase I/I clinical trials in refractory or relapsed acute myeloblastic leukemia (AML) patients.

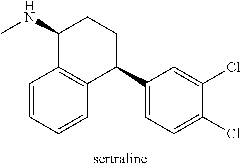 [SMILES: CN[C@H]1CC[C@@H](c2ccc(Cl)c(Cl)c2)c2ccccc21]

The applicant has discovered a novel family of compounds derived from sertraline with a strong affinity for TCTP. These compounds also possess good cytotoxicity in various human cancer cell lines.

### DISCLOSURE OF THE INVENTION

The invention relates to the compounds of formula (I) below:

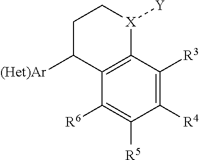

Wherein
X represents an oxygen atom, a sulfur atom, a nitrogen atom or a CH radical,
The bond X—Y and Y are absent if X represents an oxygen or sulfur atom, the bond X—Y and Y are present if X represents a nitrogen atom or a CH radical,
When present, Y represents
   a group R if X represents a nitrogen atom,
wherein R represents a hydrogen atom, a C₁ to C₆ alkyl group, an aryl group, a heteroaryl group, a (C₂-C₆)alkenyl group, a (C₂-C₆)alkynyl group or an acyl group,
   a hydrogen atom or a group —NR¹R² if X represents a CH radical, wherein R¹ and R² represent, independently of one another, a hydrogen atom, a C₁ to C₆ alkyl group, an aryl group, a heteroaryl group, a (C₂-C₆)alkenyl group, a (C₂-C₆)alkynyl group or an acyl group, or R¹ and R², together with the nitrogen atom carrying them, form a 5- or 6-membered heterocyclic ring,
(Het)Ar is an aromatic ring selected from the group consisting of aryl and heteroaryl groups,
said aromatic ring may be substituted by one or more groups selected from a halogen atom, a —COOR⁷ group, a —CONR⁸R⁹ group, a C₁ to C₆ alkyl group, a —SR¹⁰ group, a CF₃ group, a formyl group, an OR¹¹ group, a (C₂-C₆)alkenyl group,
with R⁷ representing a hydrogen atom, a C₁ to C₆ alkyl group, an aryl group, a heteroaryl group or a sugar residue,
with R⁸ and R⁹ representing, independently of one another, a hydrogen atom, a C₁ to C₆ alkyl group, an aryl group, a heteroaryl group, a (C₂-C₆)alkenyl group, a (C₂-C₆)alkynyl group, a sugar residue, an amino acid residue, a peptide residue or R⁸ and R⁹, together with the nitrogen atom carrying them, form a 5- or 6-membered heterocyclic ring,
with R¹⁰ representing a hydrogen atom, a C₁ to C₆ alkyl group, an aryl group, a heteroaryl group, a sugar residue, a peptide residue comprising at least one cysteine or —SR¹⁰ represents a cysteine residue,
with R₁₁ representing a hydrogen atom, a C₁ to C₆ alkyl group, an aryl group or a benzyl group,
R³, R⁴, R⁵, R⁶ represent, independently of one another, a hydrogen atom, a halogen atom, an —NR¹²R¹³ group, a —SR¹⁴ group, an —OR¹⁴ group or a —CF₃ group, where R¹² and R¹³ represent independently of one another a hydrogen atom, a C₁ to C₆ alkyl group, an aryl group, a heteroaryl group, a (C₂-C₆)alkenyl group, a (C₂-C₆)alkynyl group or an acyl group, or R¹² and R¹³, together with the nitrogen atom carrying them, form a 5- or 6-membered heterocyclic ring,
with R¹⁴ representing a hydrogen atom, a C₁-C₆ alkyl group, an aryl group, a heteroaryl group, a sugar residue, an amino acid residue or a peptide residue,
When Y represents a group —NR¹R², the groups —NR¹R² and (Het)Ar are in the cis-conformation,
the alcohol functions of the sugar residue and the amine functions of the amino acid residue, of the cysteine residue or of the peptide residue being in their free or protected form,

as well as their pharmaceutically acceptable salts,

with the exception of the compound of the following formula:

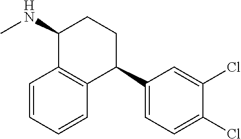 [SMILES: CN[C@H]1CC[C@@H](c2ccc(Cl)c(Cl)c2)c2ccccc21]

for use in the treatment of proliferative diseases, infectious diseases, allergies, inflammation and/or asthma.

More particularly, the compound of formula (I) according to the invention is not 4-(3,4-dichloro-phenyl)-1,2,3,4-tetrahydronaphthalene-1-ylamine or its hydrochloride salt. Preferably, the compound of formula (I) is not 4-(3,4-dichloro-phenyl)-1,2,3,4-tetrahydronaphthalene-1-ylamine or one of its pharmaceutically acceptable salts.

### DESCRIPTION OF THE FIGURES

FIG. 1: Western blot showing the expression on p53 of, from left to right, DMSO (control), sertraline at 10 μg (comparative) and the compound AC014 at 10 μg (invention) with the expression of each of these three compounds on GAPDH as a control.

FIG. 2. Diagram representing the average weight of tumors extracted from mice treated for 12 days by injection of DMSO (left column), sertraline (middle column) or the compound AC070 (right column).

### DEFINITIONS

For the purposes of the present invention, a “C₁ to C₆ alkyl” group is understood to be a monovalent saturated, linear or branched hydrocarbon chain containing 1 to 6, preferably 1 to 4, carbon atoms. Examples of such groups are methyl, ethyl, propyl, isopropyl, butyl, isobutyl, sec-butyl, tert-butyl, pentyl or hexyl.

For the purposes of the present invention, “aryl” means an aromatic hydrocarbon group, preferably containing 6 to 10 carbon atoms, and comprising one or more adjacent rings, such as, for example, a phenyl or naphthyl group. Advantageously, it is phenyl.

“Heteroaryl”, for the purposes of the present invention, means an aromatic group comprising one or more, in particular 1 or 2, fused hydrocarbon rings, wherein one or more carbon atoms, advantageously 1 to 4 and even more advantageously 1 or 2, are each replaced by a heteroatom such as, for example, a sulfur, nitrogen or oxygen atom. Examples of heteroaryl groups are furyl, thienyl, pyrrolyl, pyridyl, oxazolyl, isoxazolyl, thiazolyl, isothiazolyl, imidazolyl, pyrazolyl, oxadiazolyl, thiadiazolyl, triazolyl, tetrazolyl, pyridazinyl, pyrimidinyl, pyrazinyl, triazinyl, quinolyl, isoquinolyl, quinoxalyl, indyl, benzofuranyl or benzothiophenyl. Advantageously the heteroaryl group is selected from a pyridine, a pyrimidine, a pyrazine, a quinoline, an isoquinoline, an indole, a benzofurane and a benzothiophene.

For the purposes of the present invention, “aromatic ring” means an aryl group or a heteroaryl group as defined above.

“(C₂-C₆)alkenyl” group means, for the purposes of the present invention, a linear or branched hydrocarbon chain containing at least one double bond and comprising 2 to 6 carbon atoms. Examples include ethenyl or allyl.

“(C₂-C₆)alkynyl” group means, for the purposes of the present invention, a linear or branched hydrocarbon chain containing at least one triple bond and comprising 2 to 6 carbon atoms. Examples include ethynyl or propynyl groups.

For the purposes of the present invention, “acyl” means a C₁ to C₆ alkyl or aryl group as defined above, linked to the rest of the molecule via a carbonyl (CO) group. In particular, it can be an acetyl or benzoyl group.

For the purposes of the present invention, “5- or 6-membered heterocycle” means a 5- or 6-membered ring, saturated or unsaturated, but not aromatic, and containing one or more, advantageously 1 to 4, even more advantageously 1 or 2, heteroatoms, such as, for example, sulfur, nitrogen or oxygen atoms. These may in particular be the pyrrolidinyl, piperidinyl, piperazinyl or morpholinyl group. Advantageously, it is a pyrrolydinyl or morpholinyl group.

For the purposes of the present invention, “halogen atom” means fluorine, chlorine, bromine and iodine atoms.

For the purposes of the present invention, “sugar” means a monosaccharide or polysaccharide.

For the purposes of the present invention, “monosaccharide” means an aldose. It may include erythrose, threose, ribose, arabinose, xylose, lyxose, allose, altrose, glucose, mannose, gulose, idose, galactose, or talose, in a D or L form. It is in particular glucose.

“Polysaccharide” means, for the purposes of the present invention, a sequence of at least two monosaccharide units as defined above. In particular, it may be a disaccharide (sequence of two monosaccharide units), such as lactose.

For the purposes of the present invention, “sugar residue” means that a sugar molecule as defined above, devoid of oxygen atoms in its anomeric position, is bonded to the rest of the molecule via the carbon in the anomeric position.

For the purposes of the present invention, “amino acid” means a carboxylic acid which also has an amine functional group. In particular, all naturally occurring α-amino acids (for example alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr) and valine (Val)) in the D or L form, as well as unnatural amino acids (for example β-alanine, allylglycine, tert-leucine, 3-amino-adipic acid, 2-aminobenzoic acid, 3-aminobenzoic acid, 4-aminobenzoic acid, 2-aminobutanoic acid, 4-amino-1-carboxymethyl piperidine, 1-amino-1-cyclobutanecarboxylic acid, 4-aminocyclohexaneacetic acid, 1-amino-1-cyclohexanecarboxylic acid, (1R,2R)-2-aminocyclohexanecarboxylic acid, (1R,2S)-2-aminocyclohexanecarboxylic acid, (1S,2R)-2-aminocyclohexanecarboxylic acid, (1S,2S)-2-aminocyclohexanecarboxylic acid, 3-aminocyclohexanecarboxylic acid, 4-aminocyclohexanecarboxylic acid, (1R,2R)-2-aminocyclopentanecarboxylic acid, (1R,2S)-2-aminocyclopentanecarboxylic acid, 1-amino-1-cyclopentanecarboxylic acid, 1-amino-1-cyclopropanecarboxylic acid, 4-(2-aminoethoxy)-benzoic acid, 3-aminomethylbenzoic acid, 4-aminomethylbenzoic acid, 2-aminobutanoic acid, 4-aminobutanoic acid, 6-aminohexanoic acid, 1-aminoindane-1-carboxylic acid, 4-aminomethyl-phenylacetic acid, 4-aminophenylacetic acid, 3-amino-2-naphthoic acid, 4-aminophenylbutanoic acid, 4-amino-5-(3-indolyl)-pentanoic acid, (4R,5S)-4-amino-5-methylheptanoic acid, (R)-4-amino-5-methylhexanoic acid, (R)-4-amino-6-methylthiohexanoic acid, (S)-4-amino-pentanoic acid, (R)-4-amino-5-phenylpentanoic acid, 4-aminophenylpropionic acid, (R)-4-aminopimeric acid, (4R,5R)-4-amino-5-hyroxyhexanoic acid, (R)-4-amino-5-hydroxypentanoic acid, (R)-4-amino-5-(p-hydroxyphenyl)-pentanoic acid, 8-aminooctanoic acid, (2S,4R)-4-amino-pyrrolidine-2-carboxylic acid, (2S,4S)-4-amino-pyrrolidine-2-carboxylic acid, azetidine-2-carboxylic acid, (2S,4R)-4-benzyl-pyrrolidine-2-carboxylic acid, (S)-4,8-diaminooctanoic acid, tert-butylglycine acid, γ-carboxyglutamate, β-cyclohexylalanine, citruline, 2,3-diamino propionic acid, hippuric acid, homocyclohexylalanine, moleucine, homophenylalanine, 4-hydroxyproline, indoline-2-carboxylic acid, isonipecotic acid, α-methyl-alanine, nicopetic acid, norleucine, norvaline, octahydroindole-2-carboxylic acid, ornithine, penicillamine, phenylglycine, 4-phenyl-pyrrolidine-2-carboxylic acid, pipecolic acid, propargylglycine, 3-pyridinylalanine, 4-pyridinylalanine, 1-pyrrolidine-3-carboxylic acid, sarcosine, statins, tetrahydroisoquinoline-1-carboxylic acid, 1,2,3,4-tetrahydroisoquinoline-3-carboxylic acid, tranexamic acid).

For the purposes of the present invention, “amino acid residue” means an amino acid as defined above linked to the rest of the molecule by its amine function (NH₂), its carboxylic acid function (COOH) or any other functionality present on the amino acid such as a thiol function (SH) (for example in the case of a cysteine).

For the purposes of the present invention, “cysteine residue” means a cysteine amino acid linked to the rest of the molecule by its sulfur atom.

For the purposes of the present invention, “peptide” means a sequence of amino acids (at least two) linked together by peptide bonds (amide bonds).

For the purposes of the present invention, “peptide residue” means a peptide as defined above linked to the rest of the molecule by its amine function (NH₂), its acid function (COOH) or any other functionality present on the amino acid such as a thiol function (SH) (for example in the case of a cysteine).

“Peptide residue containing at least one cysteine” means, for the purposes of the present invention, a peptide as defined above containing at least one amino acid of the cysteine type and linked to the rest of the molecule by the thiol function (SH) of the cysteine.

For the purposes of the present invention, “alcohol function in its free form” means an —OH group.

For the purposes of the present invention, “alcohol function in its protected form” means an alcohol function (OH) wherein the hydrogen atom has been replaced by an O-protecting group.

For the purposes of the present invention, “O-protecting group” means any substituent that protects the hydroxyl or carboxyl group, i.e. a reactive oxygen atom, against adverse reactions such as the O-protecting groups described in “Greene's Protective Groups In Organic Synthesis”, 4th edition, 2007, John Wiley & Sons, Hoboken, N.J. A hydroxyl group protected by an O-protecting group may be for example an ether, ester, carbonate, acetal and similar. In particular, O-protecting groups include a (C₁-C₆)alkyl group optionally substituted by one or more (including 1 to 3) halogen atoms (such as chlorine atoms), such as methyl, ethyl, tert-butyl and 2,2,2-trichloroethyl groups; an aryl-(C₁-C₆)alkyl group, the aryl core being optionally substituted by one or more methoxy groups, such as benzyl (Bn) and p-methoxybenzyl (PMB) groups; a trityl group of formula —CAr¹Ar²Ar³, such as triphenylmethyl (also called trityl, or Tr), (4-methoxyphenyl)diphenylmethyl (also called methoxytrityl, or NMT) and bis-(4-methoxyphenyl)phenylmethyl (also called dimethoxytrityl, or DMT); a substituted methyl group of formula CH₂ORGP² or CH₂SRGP² (in particular CH₂ORGP²), such as methoxymethyl (MOM), benzyloxymethyl, 2-methoxyethoxymethyl (MEM), 2-(trimethylsilyl)ethoxymethyl and methylthiomethyl; a substituted ethyl group of formula —CH₂CH₂ORGP² or —CH₂CH₂SRGP² (in particular —CH₂CH₂ORGP²), such as ethoxyethyl (EE); a silyl group of formula —SiRGP³RGP⁴RGP⁵, such as trimethylsilyl (TMS), triethylsilyl (TES), t-butyldimethylsilyl (TBS or TBDMS) and t-butyldiphenylsilyl (TBDPS); a carbonyl group of formula CO-RGP⁶, such as acetyl (Ac), pivaloyl (Piv or Pv) and benzoyl (Bz), or of formula —CO₂—RGP⁷, such as allyloxycarbonyl (Alloc) and 9-fluorenylmethyloxycarbonyl (Fmoc); or a tetrahydropyranyl (THP) or tetrahydrofuranyl group;

with Ar¹, Ar² and Ar³ representing, independently of each other, an aryl, such as phenyl, optionally substituted by one or more methoxy groups; RGP² representing a (C₁-C₆)alkyl group (such as methyl or ethyl) optionally substituted by an aryl (such as phenyl), (C₁-C₆)alkoxy (such as methoxy) or trialkylsilyl (such as SiMe₃) group; RGP³, RGP⁴ and RGP⁵ representing, independently of one another, a (C₁-C₆)alkyl or aryl (such as phenyl) group; and RGP⁶ and RGP⁷ representing, independently of one another, a (C₁-C₆)alkyl, (C₂-C₆)alkenyl, aryl, aryl-(C₁-C₆)alkyl or 9-fluorenylmethyl group.

In particular, it is a benzyl (Bn) or acetyl (Ac) group.

“Amine function in its free form” means an —NH₂ group or an NH group which is not substituted by an N-protecting group.

For the purposes of the present invention, “amine function in its protected form” means an amine function (NH) wherein the hydrogen atom has been replaced by an N-protecting group.

For the purposes of the present invention, “N-protecting group” means any substituent that protects the NH₂ group against adverse reactions such as the N-protecting groups described in “Greene's Protective Groups In Organic Synthesis”, 4th edition, 2007, John Wiley & Sons, Hoboken, N.J. An amine function protected by an N-protecting group may be, for example, a carbamate, amide, sulfonamide, N-alkyl derivative, amino acetal derivative, N-benzyl derivative, imine derivative, enamine derivative or N-heteroatom derivative. In particular, the N-protecting groups comprise a formyl group; an aryl group, such as phenyl, optionally substituted by one or more methoxy groups, such as p-methoxyphenyl (PMP); an aryl-(C₁-C₆)alkyl group, such as benzyl, the aryl core being optionally substituted by one or more methoxy groups, such as benzyl (Bn), p-methoxybenzyl (PMB) and 3,4-dimethoxybenzyl (DMPM); a —CO-RGP⁸ group such as acetyl (Ac), pivaloyl (Piv or Pv), benzoyl (Bz) and p-methoxybenzylcarbonyl (Moz); a —CO₂-RGP⁸ group such as t-butyloxycarbonyl (Boc), trichloroethoxycarbonyl (TROC), allyloxycarbonyl (Alloc), benzyloxycarbonyl (Cbz or Z) and 9-fluorenylmethyloxycarbonyl (Fmoc); an —SO₂—RGP⁸ group such as phenylsulfonyl, tosyl (Ts or Tos) and 2-nitrobenzenesulfonyl (also called nosyl, Nos or Ns); and the like,

with RGP⁸ representing a (C₁-C₆)alkyl group optionally substituted by one or more halogen aromas such as F or Cl; a (C₂-C₆)alkenyl group such as an allyl group; an aryl group, such as phenyl, optionally substituted by one or more groups selected from OMe (methoxy) and NO₂ (nitro); an aryl-(C₁-C₆)alkyl group, such as benzyl, the aryl core being optionally substituted by one or more methoxy groups; or a 9-fluorenylmethyl group.

In particular, it is an acetyl (Ac) group.

For the purposes of the present invention, “the groups —NR¹R² and (Het)Ar are in the cis-conformation” means that the two groups —NR¹R² and (Het)Ar are located on the same side of the 1,2,3,4-tetrahydronaphthyl ring of the compound according to formula (I). Thus, when Y represents a group —NR¹R² and the groups —NR¹R² and (Het)Ar are in the cis-conformation, the compound of formula (I) according to the invention has the following formula:

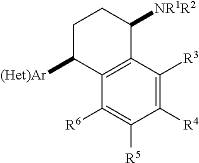

In the present invention, “pharmaceutically acceptable” means useful in preparation of a pharmaceutical composition that is generally safe, non-toxic and neither biologically nor otherwise undesirable and that is acceptable for veterinary as well as human pharmaceutical use.

“Pharmaceutically acceptable salt” of a compound means a salt that is pharmaceutically acceptable, as defined herein, and has the desired pharmacological activity of the parent compound.

Pharmaceutically acceptable salts include notably:
(1) pharmaceutically acceptable acid addition salts formed with pharmaceutically acceptable inorganic acids such as hydrochloric acid, hydrobromic acid, sulfuric acid, nitric acid, phosphoric acid and the like; or formed with pharmaceutically acceptable organic acids such as acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, mandelic acid, methanesulfonic acid, muconic acid, 2-naphthalenesulfonic acid, propionic acid, salicylic acid, succinic acid, dibenzoyl-L-tartaric acid, tartaric acid, p-toluenesulfonic acid, trimethylacetic acid, trifluoroacetic acid and the like, and
(2) pharmaceutically acceptable base addition salts formed when an acidic proton present in the parent compound is either replaced by a metal ion, for example an alkali metal ion, an alkaline earth metal ion or an aluminum ion; or coordinated with a pharmaceutically acceptable organic base such as diethanolamine, ethanolamine, N-methylglucamine,—triethanolamine, tromethamine and the like; or with a pharmaceutically acceptable inorganic base such as aluminum hydroxide, calcium hydroxide, potassium hydroxide, sodium carbonate, sodium hydroxide and the like.

Advantageously, the pharmaceutically acceptable salt is a hydrochloride.

“Proliferative disease” means a disease wherein cells proliferate in an uncontrolled manner.

“Infectious disease” means any infection, especially infections caused by parasites, also known as parasitic infections. An example of such an infection is malaria.

“Overexpressed TCTP level” means that the level of TCTP expression in the cell, particularly a cancerous cell, is higher than the level of TCTP expression in a healthy cell, particularly a non-cancerous cell.

“Management” of a disease means the prevention and/or treatment of the disease and/or its manifestations.

“4-(3,4-Dichlorophenyl)-1,2,3,4-tetrahydronaphthalene-1-ylamine” means all enantiomers and diastereomers of the molecule (including sertraline), alone or in combination.

### DETAILED DESCRIPTION OF THE INVENTION

The invention relates to the compounds of formula (I) below:

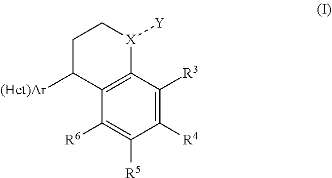

as defined above for use in the treatment of proliferative diseases, infectious diseases, allergies, inflammation and/or asthma.

Surprisingly, it was discovered that the compounds according to the invention have an affinity with TCTP. They can therefore be used in the treatment of proliferative diseases, including cancer treatment, as antitumor agents. They may also be used in the treatment of infectious diseases, in particular parasitic infectious diseases such as malaria.

In general, the compounds according to the invention may be used in the treatment or prevention of any disease or infection that requires, for its management, the inhibition of TCTP.

In addition, the compounds according to the invention have a strong affinity, being of the order of micromolar, towards TCTP. Thus, while for sertraline, the effective dose required is close to its maximum tolerated dose (MTD), the concentrations to be used in animals or humans with the compounds according to the invention can be reduced compared to the concentrations required for sertraline. The necessary effective doses are then reduced, leading to a greater difference with the maximum tolerated doses. The use of the compounds according to the invention is therefore possible in humans and animals. Moreover, this improvement in the affinity of the compounds according to the invention also makes it possible to reduce undesirable side effects. Advantageously, the affinity of the compounds according to the invention with respect to TCTP is between 1 μM and 200 μM, advantageously between 1 μM and 195 μM, preferably between 1 nM and 200 μM, advantageously between 1 nM and 195 μM, or even between 1 nM and 150 μM.

In connection with their affinity of the micromolar order for TCTP, the compounds according to the invention are also capable of inducing the overexpression of p53 (1, 5). The p53 protein is a transcription factor protein which regulates, among other things, certain important cellular functions such as mitosis or programmed death. It also plays a major role in tumor reversion. Its activation may involve either tumor reversion of cancer cells or their apoptosis. The induction of overexpression of p53 by the compounds according to the invention thus indicates that the compounds according to the invention can be used as an antitumor agent and that they appear to act in particular through a tumor reversion mechanism.

The compounds according to the invention thus present an innovative therapeutic class of antitumor agents acting in particular by a tumor reversion mechanism.

The compounds according to the invention are therefore particularly suitable for the treatment of cancer, especially cancer in which TCTP is overexpressed.

Compounds of Formula (I) for Use According to the Invention

The invention relates to the compounds of formula (I) below:

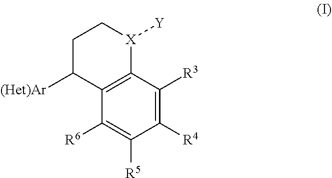

as defined above for use in the treatment of proliferative diseases, infectious diseases of allergies, inflammation and/or asthma.

The compound 4-(3,4-dichloro-phenyl)-1,2,3,4-tetrahydronaphthalene-1-ylamine or a pharmaceutically acceptable salt thereof, in particular its hydrochloride salt, is excluded from formula (I). This includes any diastereoisomer or enantiomer of this compound, alone or in a mixture. In particular, sertraline is excluded.

According to a first embodiment of the invention, X represents an oxygen atom or a sulfur atom, Y being absent.

In a first variant of the first embodiment, X represents an oxygen atom.

In a second variant of the first embodiment, X represents a sulfur atom.

According to a second embodiment of the invention, X represents a nitrogen atom or a CH radical, Y being present.

In a first variant of the second embodiment, X represents a nitrogen atom and Y is present.

Y then represents a group R. In particular R represents a hydrogen atom, a C₁ to C₆ alkyl group, or an acyl group.

In particular, R represents a hydrogen atom.

In a second variant of the second embodiment, X represents a CH radical and Y is present.

Advantageously, Y represents a hydrogen atom.

Advantageously, Y represents a group —NR¹R².

Advantageously, when R¹ or R² is an acyl group, it is selected from benzoyl and acetyl.

Advantageously, when R¹ and R² together with the nitrogen atom carrying them form a 5- or 6-membered heterocycle, —NR¹R² represents a pyrrolydine or a morpholine.

In particular, when Y represents a group —NR¹R², R¹ and/or R² represent, independently of one another, a hydrogen atom or an acyl group.

Preferably, when Y represents an —NR¹R² group, R¹ represents an acyl group, preferably an acetyl group, and advantageously R² represents a hydrogen atom.

In any one of these embodiments, according to a variant of the invention, when (Het)Ar is aryl, it is advantageously selected from phenyl and naphthyl. The aryl group thus defined may be substituted by one or more groups selected from a halogen atom, a —COOR⁷ group, a —CONR⁸R⁹ group, a C₁ to C₆ alkyl group, a —SR¹⁰ group, a CF₃ group, a formyl group, a (C₂-C₆) alkenyl group with R⁷, R⁸, R⁹ and R¹⁰ as described above, the aryl group in particular not being substituted by more than one halogen atom. In particular, the aryl group may be substituted by one or more groups selected from a halogen atom, a —COOR⁷ group, a —CONR⁸R⁹ group, a C₁ to C₆ alkyl group, a —SR¹⁰ group, and a CF₃ group, with R⁷, R⁸, R⁹ and R¹⁰ as described above, the aryl group not being substituted by more than one halogen atom.

Advantageously, when (Het)Ar is phenyl, it is unsubstituted or substituted in the meta or para position by any of the groups described above.

Advantageously, when the aryl is substituted with a halogen atom, the halogen atom is selected from fluorine, chlorine, bromine or iodine. In particular, it is selected from a chlorine, bromine and iodine atom.

In any one of these embodiments, according to a variant of the invention, when (Het)Ar is a heteroaryl, it is selected from a pyridine, a pyrimidine, a pyrazine, a quinoline, an isoquinoline, an indole, a benzofurane and a benzothiophene. The heteroaryl group thus defined may be substituted by one or more groups selected from a halogen atom, a —COOR⁷ group, a —CONR⁸R⁹ group, a C₁ to C₆ alkyl group, a —SR¹⁰ group, a CF₃ group, a formyl group, a (C₂-C₆) alkenyl group, with R⁷, R⁸, R⁹ and R¹⁰ as described above, the heteroaryl group in particular not being substituted by more than one halogen atom. In particular, the aryl group may be substituted by one or more groups selected from a halogen atom, a —COOR⁷ group, a —CONR⁸R⁹ group, a C₁-C₆ alkyl group, a —SR¹⁰ group, and a CF₃ group, with R⁷, R⁸, R⁹ and R¹⁰ as described above, the heteroaryl group in particular not being substituted by more than one halogen atom. In particular, the heteroaryl is unsubstituted.

In any one of the embodiments and variants of the invention, advantageously, according to another variant of the invention, the aromatic ring Het(Ar) is substituted by one or more groups selected from a halogen atom, a —COOR⁷ group, and a —CONR⁸R⁹ group with R⁷, R⁸ and R⁹ as described above, the aromatic ring Het(Ar) not being substituted by more than one halogen atom.

In any one of the embodiments and variants of the invention, advantageously, when the aromatic ring Het(Ar) is substituted by a halogen atom, the halogen atom is selected from a fluorine, chlorine, bromine and iodine atom. In particular, it is selected from a chlorine, bromine and iodine atom, preferably it is selected from a bromine and iodine atom.

In any one of the embodiments and variants of the invention, when the aromatic ring is substituted by a —COOR⁷ group, R⁷ advantageously represents a methyl group, an ethyl group or an isopropyl group.

In any one of the embodiments and variants of the invention, when the aromatic ring is substituted by a —CONR⁸R⁹ group and when R⁸ and/or R⁹ represent a sugar residue, the sugar is advantageously selected from glucose, mannose, arabinose or galactose. When R⁸ and R⁹ together with the nitrogen atom carrying them form a 5- or 6-membered heterocycle, —NR⁸R⁹ advantageously represents a pyrrolydine or a morpholine.

In any one of the embodiments and variants of the invention, when the aromatic ring is substituted by a group —SR¹⁰ and when R¹⁰ represents a sugar residue, the sugar is advantageously selected from glucose, mannose, arabinose or galactose.

In any one of the embodiments and variants of the invention, advantageously, according to another variant of the invention, the groups R³, R⁴, R⁵, R⁶ represent a hydrogen atom.

In any one of the modes of execution and variants of the invention, advantageously, according to another variant of the invention, R³, R⁴, R⁵, R⁶ represent, independently of one another, a hydrogen atom, a halogen atom, a —NR¹²R¹³ group, a —SR¹⁴ group, a —OR¹⁴ group, a —CF₃ group.

Advantageously R¹⁴ represents a methyl group.

Advantageously, when R¹⁴ represents a sugar residue, the sugar is selected from glucose, mannose, arabinose or galactose.

Compounds of Formula (II) for Use According to the Invention

Compounds of formula (I) preferred according to the invention are compounds of the following formula (II):

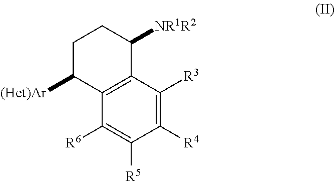

wherein R¹, R², R³, R⁴, R⁵, R⁶ and Het(Ar) are as previously defined for compounds of formula (I),

for use in the treatment of proliferative diseases, in particular cancer, infectious diseases, allergies, inflammation and/or asthma.

In particular, R¹ and/or R² represent, independently of one another, a hydrogen atom or an acyl group.

Advantageously, when R¹ or R² is an acyl group, it is selected from benzoyl and acetyl.

Preferably R¹ represents an acyl group, preferably an acetyl group, and advantageously R² represents a hydrogen atom.

According to a variant of the invention for the compounds of formula (II), when (Het)Ar is aryl, it is advantageously selected from phenyl or naphthyl. The aryl group thus defined may be substituted by one or more groups selected from a halogen atom, a —COOR⁷ group, a —CONR⁸R⁹ group, a C₁ to C₆ alkyl group, a —SR¹⁰ group, a CF₃ group, a formyl group, a (C₂-C₆) alkenyl group with R⁷, R⁸, R⁹ and R¹⁰ as described above, the aryl group in particular not being substituted by more than one halogen atom. In particular, the aryl group may be substituted by one or more groups selected from a halogen atom, a —COOR⁷ group, a —CONR⁸R⁹ group, a C₁ to C₆ alkyl group, a —SR¹⁰ group, and a CF₃ group, with R⁷, R⁸, R⁹ and R¹⁰ as described above, the aryl group in particular not being substituted by more than one halogen atom.

Advantageously, when (Het)Ar is phenyl, it is unsubstituted or substituted in the meta or para position by any of the groups described above.

When the aryl is substituted with a halogen atom, the halogen atom is advantageously selected from fluorine, chlorine, bromine and iodine. In particular, it is selected from a chlorine, bromine and iodine atom, preferably it is selected from a bromine and iodine atom, preferably it is selected from a bromine and iodine atom.

According to a variant of the invention for the compounds of formula (II), when (Het)Ar is a heteroaryl, it is advantageously selected from a pyridine, a pyrimidine, a pyrazine, a quinoline, an isoquinoline, an indole, a benzofurane and a benzothiophene.

The heteroaryl group thus defined may be substituted by one or more groups selected from a halogen atom, a —COOR⁷ group, a —CONR⁸R⁹ group, a C₁ to C₆ alkyl group, a —SR¹⁰ group, a CF₃ group, a formyl group, a (C₂-C₆) alkenyl group with R⁷, R⁸, R⁹ and R¹⁰ as described above, the heteroaryl group in particular not being substituted by more than one halogen atom. In particular, the heteroaryl group may be substituted by one or more groups selected from a halogen atom, a —COOR⁷ group, a —CONR⁸R⁹ group, a C₁ to C₆ alkyl group, a —SR¹⁰ group, and a CF₃ group, with R⁷, R⁸, R⁹ and R¹⁰ as described above, the heteroaryl group being in particular not substituted by more than one halogen atom. In particular, the heteroaryl is unsubstituted.

In any one of the variants of the invention for the compounds of formula (II), advantageously, according to another variant of the invention, the aromatic ring Het(Ar) is substituted by one or more groups selected from a halogen atom, a —COOR⁷ group, and a —CONR⁸R⁹ group with R⁷, R⁸ and R⁹ as described above, the aromatic ring Het(Ar) not being substituted by more than one halogen atom.

In any one of the variants of the invention for compounds of formula (II), when the aromatic ring Het(Ar) is substituted by a halogen atom, the halogen atom is advantageously selected from a fluorine, chlorine, bromine and iodine atom. In particular, it is selected from a chlorine, bromine and iodine atom, preferably it is selected from a bromine and iodine atom.

In any one of the variants of the invention for compounds of formula (II), when the aromatic ring is substituted by a —COOR⁷ group, R⁷ advantageously represents a methyl group, an ethyl group or an isopropyl group.

In any one of the variants of the invention for compounds of formula (II), when the aromatic ring is substituted by a —CONR⁸R⁹ group and when R⁸ and/or R⁹ represent a sugar residue, the sugar is advantageously selected from glucose, mannose, arabinose or galactose.

When R⁸ and R⁹ together with the nitrogen atom carrying them form a 5- or 6-membered heterocycle, —NR⁸R⁹ advantageously represents a pyrrolydine or a morpholine.

In any one of the variants of the invention for compounds of formula (II), when the aromatic ring is substituted by a group —SR¹⁰ and when R¹⁰ represents a sugar residue, the sugar is advantageously selected from glucose, mannose, arabinose or galactose.

In any one of the variants of the invention for the compounds of formula (II), advantageously, according to a variant of the invention, the groups R³, R⁴, R⁵, R⁶ represent a hydrogen atom.

In any one of the variants of the invention for the compounds of formula (II), advantageously, according to another variant of the invention, R³, R⁴, R⁵, R⁶ represent, independently of one another, a hydrogen atom, a halogen atom, a —NR¹²R¹³ group, a —SR¹⁴ group, a —OR¹⁴ group, a —CF₃ group.

Advantageously R¹⁴ represents a methyl group.

Advantageously, when R¹⁴ represents a sugar residue, the sugar is selected from glucose, mannose, arabinose or galactose.

In particular, Het(Ar) represents a naphthalene or a phenyl substituted by a bromine atom, an iodine atom or a —COOiPr group.

Compounds of Formula (III) for Use According to the Invention

In another embodiment, advantageous compounds of formula (I) according to the invention are compounds of the following formula (III):

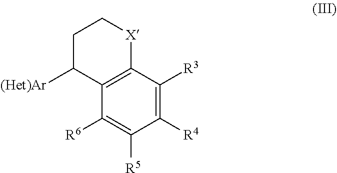

Wherein
X′ represents CH₂, O, S or N—R,
R, R³, R⁴, R⁵, R⁶ and Het(Ar) are as previously defined for the compounds of formula (I),

for use in the treatment of proliferative diseases, in particular cancer, infectious diseases, allergies, inflammation and/or asthma.

According to a first embodiment of the invention for the compounds of formula (III), X′ represents an oxygen atom or a sulfur atom.

In a first variant of this first embodiment, X′ represents an oxygen atom.

In a second variant of this first embodiment, X′ represents a sulfur atom.

According to a second embodiment of the invention for the compounds of formula (III), X′ represents N—R or CH₂.

In a first variant of this second embodiment, X′ represents N—R. In particular R represents a hydrogen atom, a C₁ to C₆ alkyl group, or an acyl group.

In particular, R represents a hydrogen atom and X′ therefore represents NH.

In a second variant of this second embodiment, X′ represents CH₂.

According to a variant of the invention for the compounds of formula (III), when (Het)Ar is aryl, it is advantageously selected from phenyl or naphthyl. The aryl group thus defined may be substituted by one or more groups selected from a halogen atom, a —COOR⁷ group, a —CONR⁸R⁹ group, a C₁ to C₆ alkyl group, a —SR¹⁰ group, a CF₃ group, a formyl group, a (C₂-C₆) alkenyl group, with R⁷, R⁸, R⁹ and R¹⁰ as described above, the aryl group in particular not being substituted by more than one halogen atom. In particular, the aryl group may be substituted by one or more groups selected from a halogen atom, a —COOR⁷ group, a —CONR⁸R⁹ group, a C₁ to C₆ alkyl group, a —SR¹⁰ group, and a CF₃ group, with R⁷, R⁸, R⁹ and R¹⁰ as described above, the aryl group in particular not being substituted by more than one halogen atom.

Advantageously, when (Het)Ar is phenyl, it is unsubstituted or substituted in the meta or para position by any of the groups described above.

When aryl is substituted with a halogen atom, the halogen atom is advantageously selected from fluorine, chlorine, bromine and iodine. In particular, it is selected from a chlorine, bromine and iodine atom, preferably it is selected from a bromine and iodine atom.

According to a variant of the invention for the compounds of formula (III), when (Het)Ar is a heteroaryl, it is advantageously selected from a pyridine, a pyrimidine, a pyrazine, a quinoline, an isoquinoline, an indole, a benzofurane and a benzothiophene. The heteroaryl group thus defined may be substituted by one or more groups selected from a halogen atom, a —COOR⁷ group, a —CONR⁸R⁹ group, a C₁ to C₆ alkyl group, a —SR¹⁰ group, a CF₃ group, a formyl group, a (C₂-C₆) alkenyl group, with R⁷, R⁸, R⁹ and R¹⁰ as described above, the heteroaryl group in particular not being substituted by more than one halogen atom. In particular, the heteroaryl group may be substituted by one or more groups selected from a halogen atom, a —COOR⁷ group, a —CONR⁸R⁹ group, a C₁ to C₆ alkyl group, a —SR¹⁰ group, and a CF₃ group, with R⁷, R⁸, R⁹ and R¹⁰ as described above, the heteroaryl group in particular not being substituted by more than one halogen atom. In particular, the heteroaryl is unsubstituted.

In any one of the embodiments and variants of the invention for the compounds of formula (III), advantageously, according to another variant of the invention, the aromatic ring Het(Ar) is substituted by one or more groups selected from a halogen atom, a —COOR⁷ group, and a —CONR⁸R⁹ group with R⁷, R⁸ and R⁹ as described above, the aromatic ring Het(Ar) not being substituted by more than one halogen atom.

In any one of the embodiments and variants of the invention for the compounds of formula (III), when the aromatic ring is substituted by a halogen atom, the halogen atom is advantageously selected from a fluorine, chlorine, bromine and iodine atom. In particular, it is selected from a chlorine, bromine and iodine atom, preferably it is selected from a bromine and iodine atom.

In any one of the embodiments and variants of the invention for the compounds of formula (III), when the aromatic ring Het(Ar) is substituted by a —COOR⁷ group, R⁷ advantageously represents a methyl group, an ethyl group or an isopropyl group.

In any one of the embodiments and variants of the invention for the compounds of formula (III), when the aromatic ring Het(Ar) is substituted by a —CONR⁸R⁹ group and when R⁸ and/or R⁹ represent a sugar residue, the sugar is advantageously selected from glucose, mannose, arabinose or galactose. When R⁸ and R⁹ together with the nitrogen atom carrying them form a 5- or 6-membered heterocycle, —NR⁸R⁹ represents in particular a pyrrolydine or a morpholine.

In any one of the embodiments and variants of the invention for the compounds of formula (III), when the aromatic ring Het(Ar) is substituted by an —SR¹⁰ group and when R¹⁰ represents a sugar residue, the sugar is advantageously selected from glucose, mannose, arabinose or galactose.

In any one of the embodiments and variants of the invention for the compounds of formula (III), advantageously, according to a variant of the invention, the groups R³, R⁴, R⁵, R⁶ represent a hydrogen atom.

In any one of the embodiments and variants of the invention for the compounds of formula (III), advantageously, according to another variant of the invention, R³, R⁴, R⁵, R⁶ represent, independently of one another, a hydrogen atom, a halogen atom, a —NR¹²R¹³ group, a —SR¹⁴ group, a —OR¹⁴ group, a —CF₃ group. Advantageously R¹⁴ represents a methyl group.

Advantageously, when R¹⁴ represents a sugar residue, the sugar is selected from glucose, mannose, arabinose or galactose.

The compounds of the present invention for use in the treatment of proliferative diseases, in particular cancer, infectious diseases, in particular parasitic infectious diseases, allergies, inflammation and/or asthma are preferably selected from

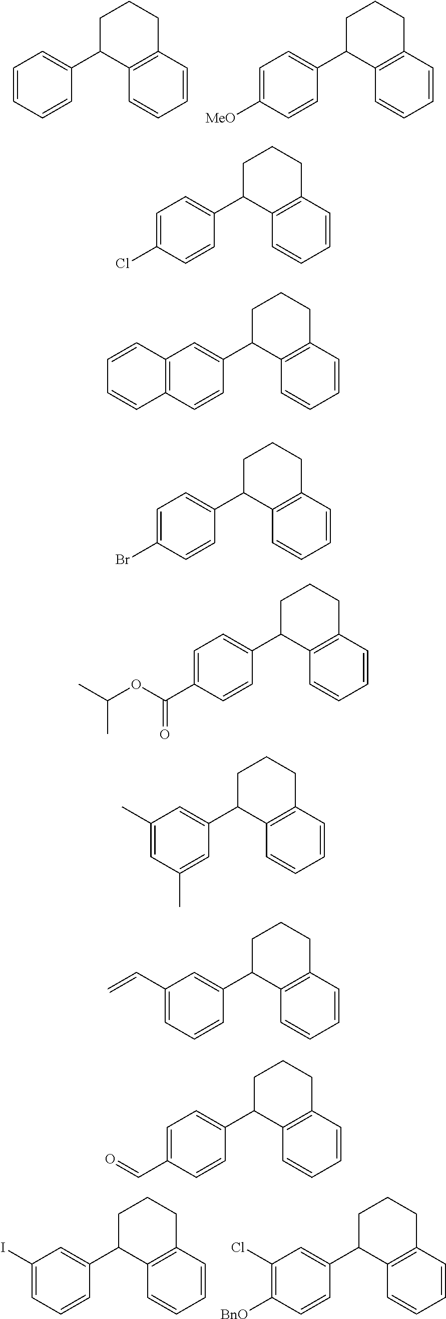

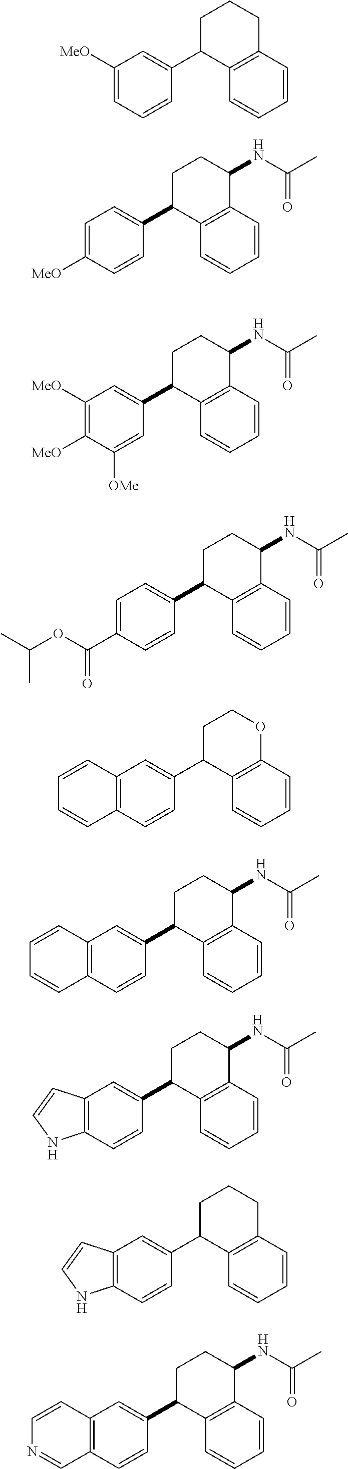

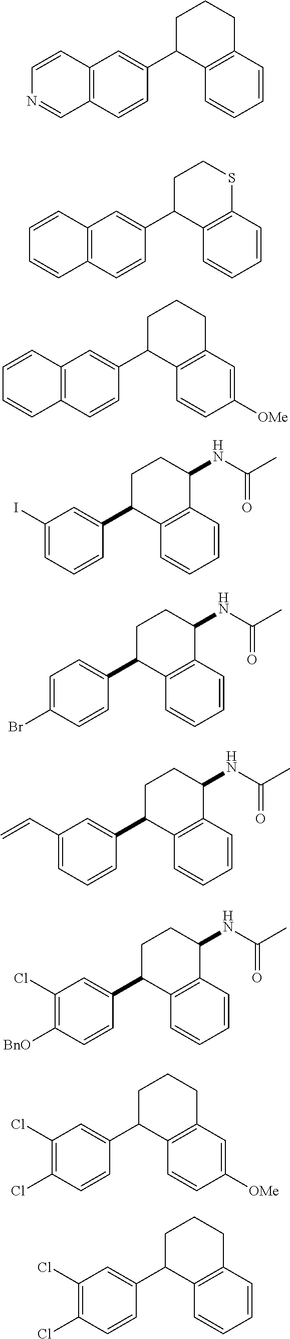

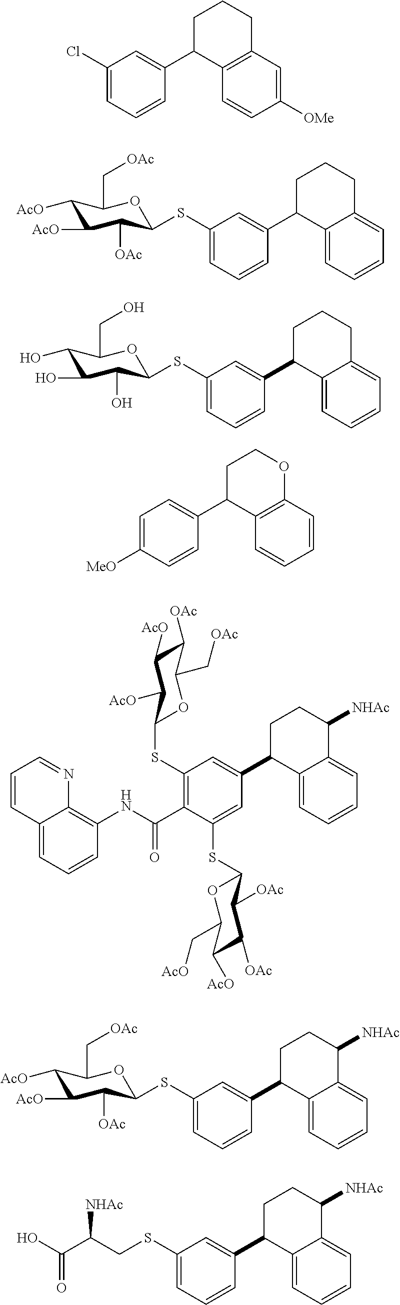

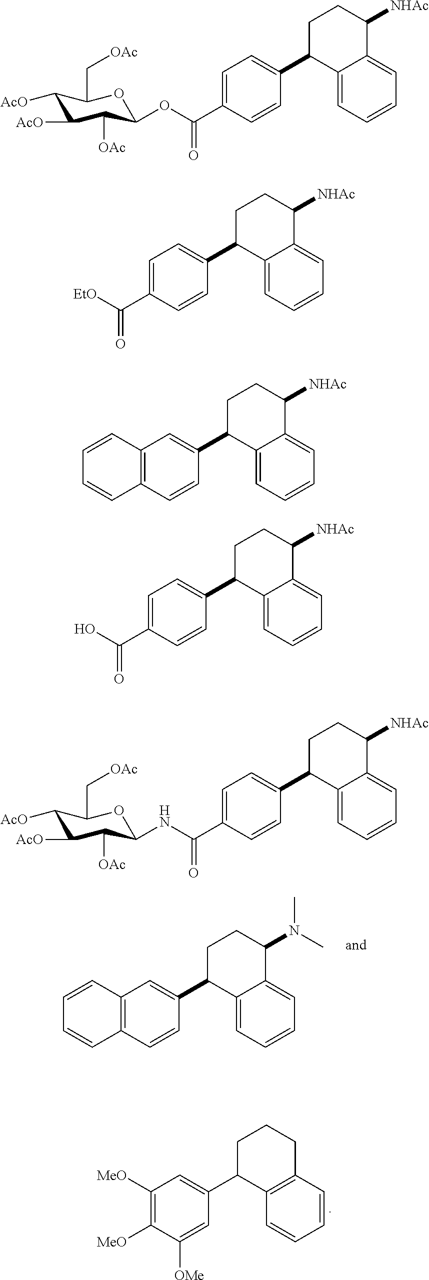

and their pharmaceutically acceptable salts, such as hydrochlorides.

The compounds of the present invention for use in the treatment of proliferative diseases, in particular cancer, infectious diseases, in particular parasitic infectious diseases, allergies, inflammation and/or asthma are preferably selected from

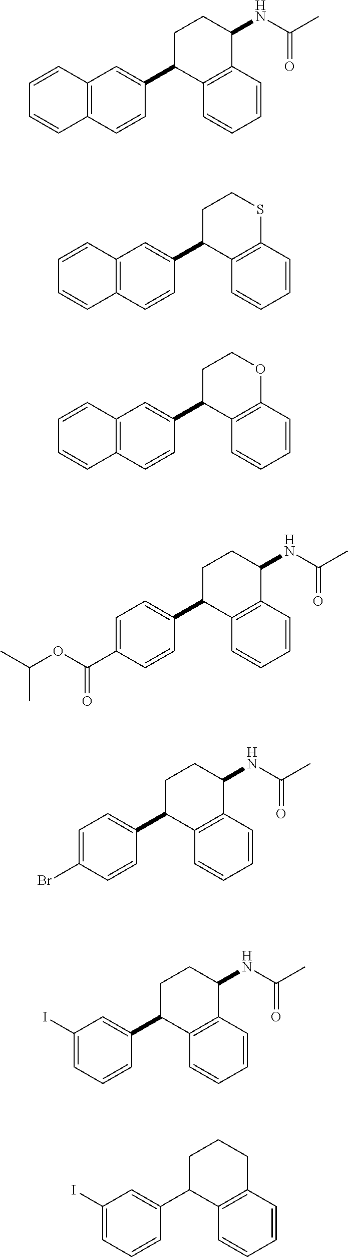

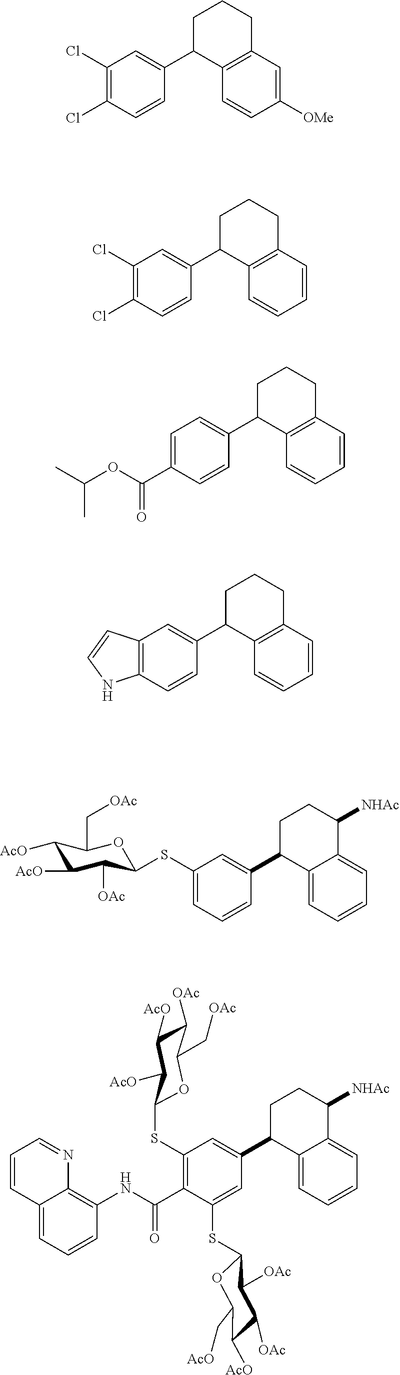

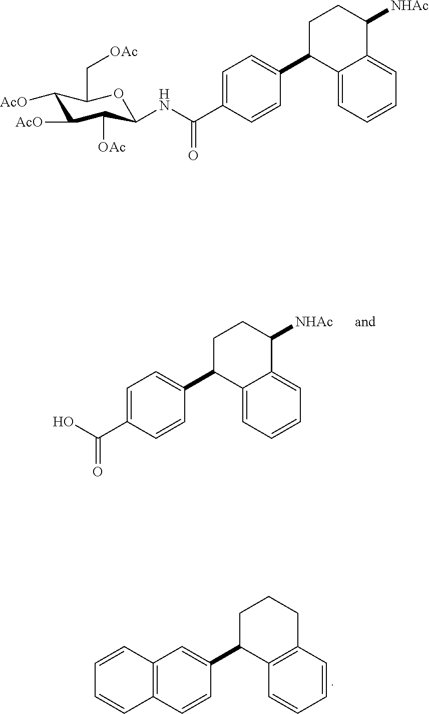

and their pharmaceutically acceptable salts, such as hydrochlorides.

Uses According to the Present Invention

The invention relates to the compounds according to the invention which can be used in the treatment of any disease which requires the inhibition of TCTP for its management.

In a first variant, the disease is a proliferative disease such as cancer, psoriasis, preferably cancer.

In a second variant, the disease is an infectious disease, in particular a parasitic infectious disease such as malaria (8).

In a third variant, the disease is an allergy, inflammation or asthma (9-14).

In particular, the invention relates to compounds according to the invention which can be used in the treatment of proliferative diseases, in particular cancer and psoriasis, more particularly cancer, the treatment of infectious diseases, in particular malaria, and/or the treatment of allergies, inflammation and/or asthma. More particularly, the invention relates to the compounds according to the invention for use in the treatment of cancer or malaria.

Advantageously, the invention relates to compounds according to the invention which can be used in the treatment of cancer by inhibition of TCTP, in particular in the treatment of cancer by tumor reversion.

Indeed, the compounds according to the invention exhibit an improved inhibition of TCTP compared to sertraline and induce an overexpression of the p53 protein. The inhibition of TCTP and overexpression of p53 protein are, among other things, two characteristics of the tumor reversion mechanism.

In particular, the invention relates to compounds according to the invention for use in the treatment of cancer, in particular cancer wherein TCTP is overexpressed. Among the cancers are, without limitation, leukemia, lymphoma, sarcoma, liver cancer, pancreatic cancer, lung cancer, stomach cancer, esophageal cancer, kidney cancer, pleural cancer, thyroid cancer, skin cancer, cervical cancer breast cancer, ovarian cancer, colon cancer, testicular cancer, prostate cancer, brain cancer, rectal cancer, or bone cancer.

In particular the invention relates to compounds according to the invention for use in the treatment of cancer when the cancer is acute myeloid leukemia, breast cancer, sarcoma, colon cancer, lung cancer, melanoma or brain cancer.

According to a particular embodiment of the invention, the compounds for use according to the invention are administered in association with another active agent, in particular an anticancer compound, whether cytotoxic or not.

Without limitation, the active agents which may be associated with the compounds for use according to the present invention may be selected from 6-mercaptopurine, fludarabine, cladribine, pentostatin, cytarabine, 5-fluorouracil, gemcitabine, methotrexate, raltitrexed, irinotecan, topotecan, etoposide, daunorubicin, doxorubicin, epirubicin, idarubicin, pirarubicin, mitoxantrone, chlormethine, cyclophosphamide, ifosfamide, melphalan, chlorambucil, busulfan, carmustine, fotemustine, streptozocin, carboplatin, cisplatin, oxaliplatin, procarbazine, dacarbazine, bleomycin, vinblastine, vincristine, vindesine, vinorelbine, paclitaxel, docetaxel, L-asparaginase, flutamide, nilutamide, bicalutamide, cyproterone acetate, triptorelin, leuprorelin, goserelin, buserelin, formestane, aminoglutethimide, anastrazole, letrozole, tamoxifen, octreotide, lanreotide, (Z)-3-[2,4-dimethyl-5-(2-oxo-1,2-dihydro-indol-3-ylidenemethyl)-1H-pyrrol-3-yl]-propionic acid, 4-((9-chloro-7-(2,6-difluorophenyl)-5H-pyrimidol(5,4-d)(2)benzazepin-2-yl)amino)benzoic acid, 5,6-dimethylxanthenone-4-acetic acid, 3-(4-(1,2-diphenylbut-1-enyl)phenyl)acrylic acid, imatinib, erlotinib, sunitinib, sorafenib, lapatenib, dasatinib, trastuzumab, cetuximab, and rituximab.

In particular, the compounds for use according to the invention are administered in combination with cytarabine.

A pharmaceutical composition comprising:
(i) at least one compound of the present invention,
(ii) at least one other active agent,

as combination products for simultaneous, separate or sequential use can be used for cancer treatment.

The active agent(s) may be as listed above. In particular, the active agent may be cytarabine.

According to a particular embodiment of the invention, the compounds for use according to the invention are administered during or possibly before and after radiotherapy.

The compounds according to the present invention may be administered by any usual route, in particular by oral, sublingual, parenteral subcutaneous, intramuscular, intravenous, transdermal, local, cutaneous, mucosal or rectal administration.

Compounds according to the present invention may be used in doses between 0.01 mg and 1000 mg per day, given in a single dose once a day or preferably administered in several doses throughout the day, for example twice a day in equal doses. The dose administered per day is advantageously between 5 mg and 500 mg, even more advantageously between 10 mg and 200 mg. It may be necessary to use doses outside these ranges, which practitioner can determine for themselves.

The present invention also relates to a method for inhibiting TCTP comprising administering to a patient in need thereof a compound of the present invention, alone or in association, advantageously synergistically, with at least one other active agent as defined above.

The present invention relates in particular to a method of treating proliferative diseases, especially in the treatment of cancer, comprising the administration to a patient in need thereof of a compound of the present invention, alone or in association, advantageously synergistic, with at least one other active agent as defined above.

The present invention relates in particular to a method of treating infectious diseases, in particular parasitic infectious diseases such as malaria, comprising the administration to a patient in need thereof of a compound of the present invention, alone or in association, advantageously synergistic, with at least one other active agent as defined above.

The present invention relates in particular to a method of treating allergies, inflammation and/or asthma, comprising administering to a patient in need thereof a compound of the present invention, alone or in association, advantageously synergistically, with at least one other active agent as defined above.

The present invention also relates to the use of a compound of the present invention for the preparation of a medicinal product for the treatment of proliferative diseases and infectious diseases, in particular for the treatment of cancer.

The present invention relates in particular to the use of a compound of the present invention for the preparation of a medicinal product for the treatment of proliferative diseases, in particular in the treatment of cancer.

The present invention relates in particular to the use of a compound of the present invention for the preparation of a medicinal product for the treatment of parasitic infectious diseases, in particular for the treatment of malaria.

The present invention relates in particular to the use of a compound of the present invention for the preparation of a medicinal product for the treatment of allergies, inflammation and/or asthma.

In particular, the patient in need of treatment is a mammal, in particular a human.

Novel Compounds According to the Invention

The invention also relates to the novel compounds selected from:

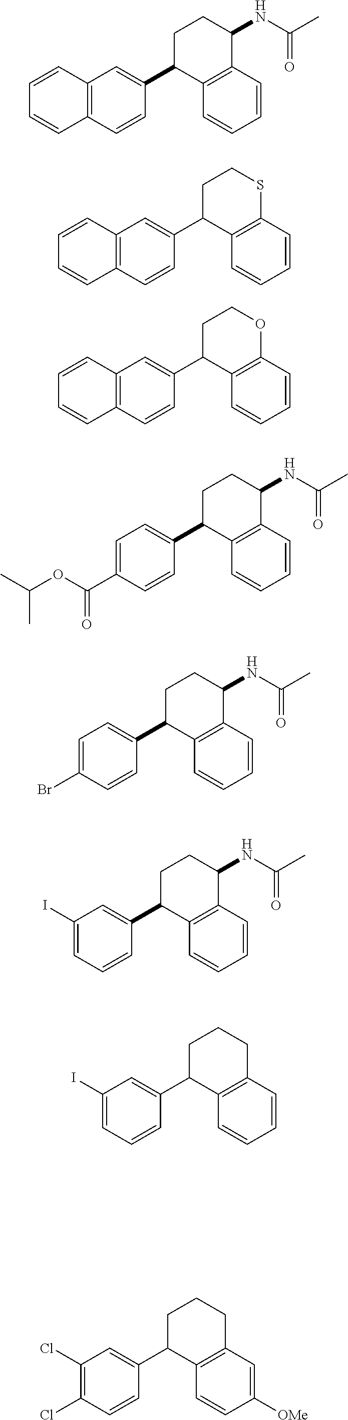

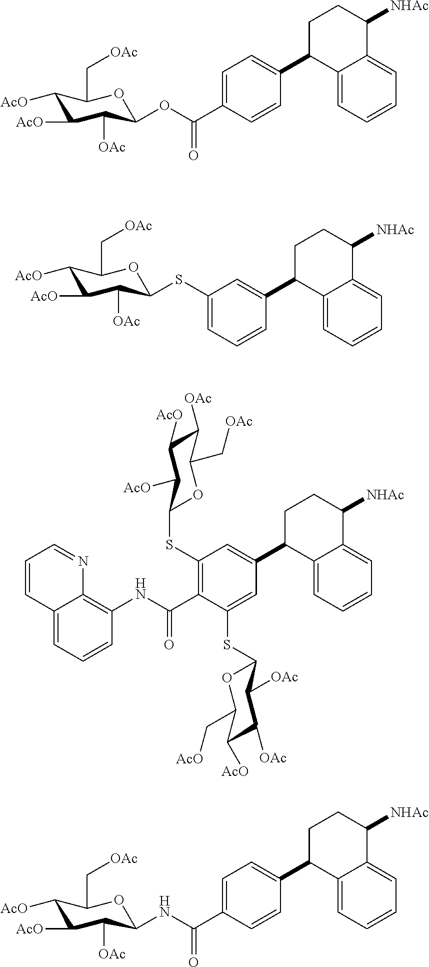

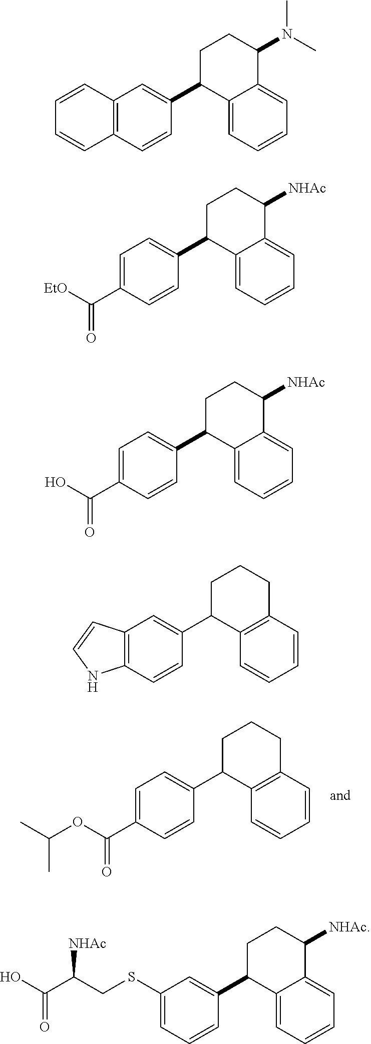

and their pharmaceutically acceptable salts.

In particular, the novel compounds are selected from:

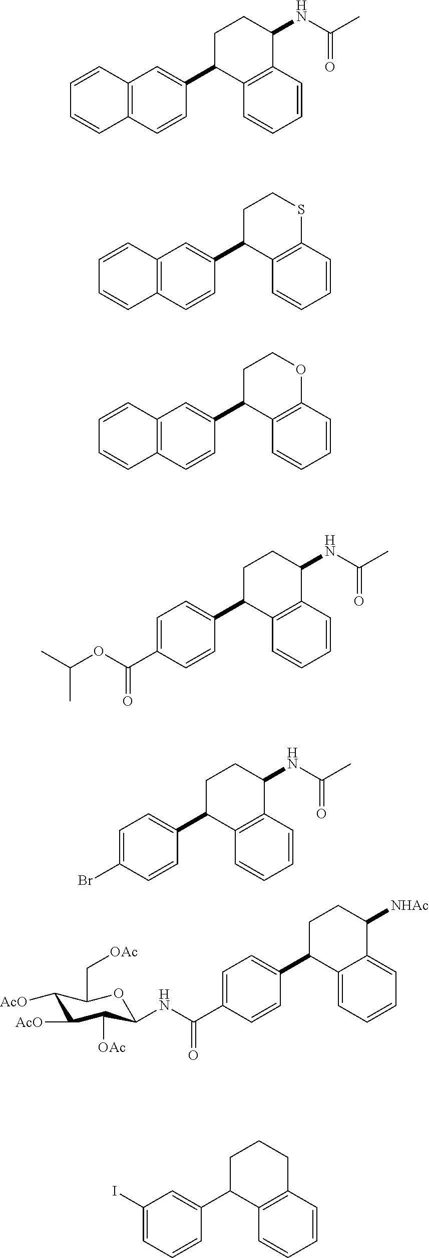

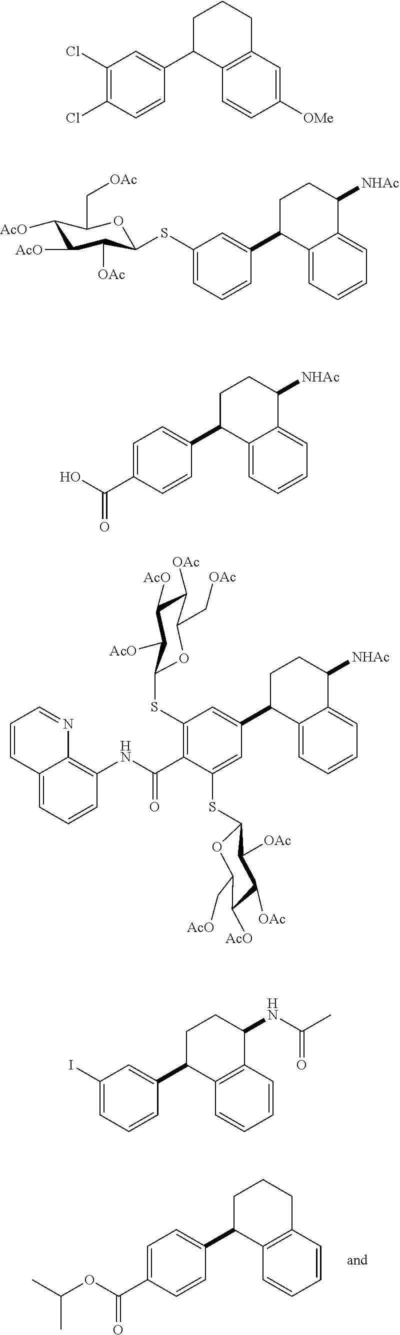

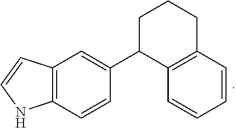 [SMILES: c1ccc2c(c1)CCCC2c1ccc2[nH]ccc2c1]

and their pharmaceutically acceptable salts.

Pharmaceutical Compositions According to the Invention

The invention also relates to a pharmaceutical composition for use in the treatment of proliferative diseases, infectious diseases, allergies, inflammation and/or asthma comprising a compound of formula (I), (II) or (III) according to the invention, according to any one of the embodiments described above, and a pharmaceutically acceptable excipient. The pharmaceutical compositions according to the invention may be intended for enteral (for example oral) or parenteral (for example intravenous) administration, preferably oral or intravenous administration. The active agent may be administered in unit dose forms for administration, mixed with conventional pharmaceutical carriers, to animals, preferably mammals, including man.

For oral administration, the pharmaceutical composition may be in solid or liquid form (solution or suspension).

A solid composition may be in the form of tablets, capsules, powders, granules and the like. In tablets, the active agent may be mixed with one or more pharmaceutical carrier(s) such as gelatin, starch, lactose, magnesium stearate, talc, gum arabic and the like, before being compressed. In addition, the tablets may be coated, in particular with sucrose or other suitable materials, or they may be treated in such a way that they have a prolonged or delayed activity. In powders or granules, the active agent may be mixed or granulated with dispersing, wetting or suspending agents and with flavor enhancers or sweeteners. In capsules, the active agent may be introduced in soft or hard capsules in the form of a powder or granules as mentioned above or in the form of a liquid composition as mentioned below.

A liquid composition may contain the active agent with a suitable sweetener, flavor enhancer or coloring agent in a solvent such as water. The liquid composition may also be obtained by suspending or dissolving a powder or granules, as mentioned above, in a liquid such as water, juice, milk, etc. This may be a syrup or an elixir, for example.

For parenteral administration, the composition may be in the form of an aqueous suspension or solution which may contain suspending and/or wetting agents. The composition is advantageously sterile. It may be in the form of an isotonic solution (especially in relation to blood).

The compounds according to the invention can be used in a pharmaceutical composition at a dose ranging from 0.01 mg to 1000 mg per day, administered in a single dose once a day or in several doses during the day, for example twice a day in equal doses. The dose administered daily is advantageously between 5 mg and 500 mg, and more advantageously between 10 mg and 200 mg. However, it may be necessary to use doses outside these ranges, which the skilled person can determine.

Process for the Synthesis of Compounds According to the Present Invention

The compounds according to the invention are obtained by short synthesis processes compatible with industrial requirements.

Through a convergent synthesis strategy, the synthesis pathway implements the key reaction between a tosylhydrazone of formula A and a boronic acid of formula B or an aryl iodide of formula C:

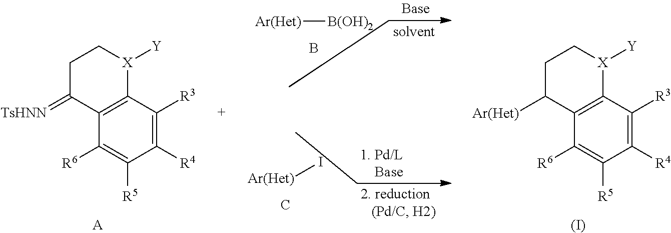

Advantageously, the compounds according to the invention can be prepared by a process comprising a step of coupling the compound of formula A, wherein X, Y, R³, R⁴, R⁵ and R⁶ are as previously defined, with the compound of formula B (Het)ArB(OH)₂ or the compound of formula C (Het)Ar—I, wherein (Het)Ar is as previously defined.

In particular, the compounds according to the invention can be prepared by a process comprising the following successive steps:
a) Reaction of tosylhydrazine with a compound of formula A′

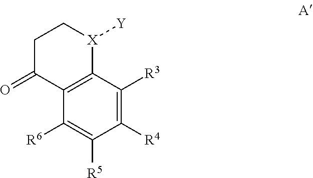

wherein X, Y, R³, R⁴, R⁵ and R⁶ are as defined above, to yield the tosylhydrazone compound of formula A,

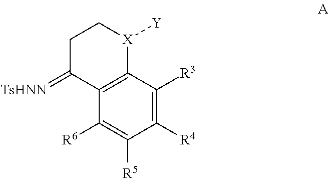

b) Coupling the compound of formula A with the compound of formula B (Het)ArB(OH)₂ or the compound of formula C (Het)Ar—I, wherein (Het)Ar is as previously defined
c) Possibly salification to give a pharmaceutically acceptable salt of the compound according to the invention
d) Separation of the compound according to the invention or its pharmaceutically acceptable salt from the reaction medium resulting from the coupling.

Compounds of formula (I), (II) or (III) are thus obtained by this process. The person skilled in the art will know which groups of (Het)Ar, X, Y, R³, R⁴, R⁵ and R⁶ as defined above are compatible with the coupling and which groups must first be protected and according to which method. The compounds of formula (I) can then be subjected to several transformations by means of processes known to the person skilled in the art in order to access other compounds according to formula (I) which are variously functionalized. The starting products of formulas A′, B, and C may be commercially available or prepared by methods known to the skilled person.

In particular, the coupling of the compound of formula A with the compound of formula B (Het)ArB(OH)₂ takes place in the presence of a base. Said base may be potassium carbonate, cesium carbonate, sodium carbonate, sodium tert-butoxide, potassium tert-butoxide, sodium methoxide or potassium methoxide.

In particular, the coupling of the compound of formula A with the compound of formula B (Het)ArB(OH)₂ takes place in a polar solvent, such as dioxane.

Advantageously, the coupling of the compound of formula A with the compound of formula C (Het)Ar—I takes place in the presence of a Pd/L catalyst system and a base. The catalyst system may be in the form of a complex of palladium and a ligand or a precatalyst. In particular, the Pd/L catalyst system can be Pd(OAc)₂/XPhos, Pd(OAc)₂/DPPE, Pd₂(dba)₃/XPhos. The base may be potassium carbonate, cesium carbonate, sodium carbonate, sodium tert-butoxide, potassium tert-butoxide, or triethylamine.

In particular, the coupling of the compound of formula A with the compound of formula C (Het)Ar—I takes place in a solvent, such as dioxane, THF or toluene.

### BIBLIOGRAPHICAL REFERENCES

1. Amson, R., Karp, J. E., and Telerman, A. Current Opinion in Oncology 2013, 25, 59-65.
2. Amson, R., Pece, S., Marine, J. C., Di Fiore, P. P., and Telerman, A. Trends in cell biology 2013, 23, 37-46.
3. Telerman, A., and Amson, R. Nature Reviews 2009, 9, 206-216.
4. Tuyunder, M., Fiucci, G., Prieur, S., Lespagnol, A., Geant, A., Beaucourt, S., Duflaut, D., Besse, S., Susini, L., Cavalleri, J., Moras, D., Amson, R., and Telerman, A. Proceedings of the national Academy of Science of the U.S. Pat. No. 2,004,101, 15364-15369.
5. Amson, R., Pece, S., Lespagnol, A., Vyas, R., Mazzarol, G., Tosoni, D., Colaluca, I., Viale, G., Rodirigues-Ferreira, S., Wynendaele, J., Chaloin, O., Hoebeke, J., Marine, J. C., Di Fiore, P. P., and Telerman, A. Nature Medicine, 2012, 18, 91-100.
6. Maeng J and Lee K, Pharm Anal Acta, 2014, 5:7
7. B. Calderon-Pérez, B. Xoconostle-Cazares, R. Lira-Carmona, R. Hernandez-Rivas, J. Ortega-Lopez, R. Ruiz-Medrano, Plos One, 2014, 9(1) e85514
8. MacDonald, S. M., Bhisutthibhan, J., Shapiro, T. A., Rogerson, S. J., Taylor, T. E., Tembo, M., Langdon, J. M., and Meshnick, S. R. (2001). Immune mimicry in malaria: Plasmodium falciparum secretes a functional histamine-releasing factor homolog in vitro and in vivo. Proceedings of the National Academy of Sciences of the United States of America 98, 10829-10832.
9. MacDonald, S. M., Rafnar, T., Langdon, J., and Lichtenstein, L. M. (1995). Molecular identification of an IgE-dependent histamine-releasing factor. Science 269, 688-690.
10. Yeh, Y. C., Xie, L., Langdon, J. M., Myers, A. C., Oh, S. Y., Zhu, Z., and Macdonald, S. M. (2010). The effects of overexpression of histamine releasing factor (HRF) in a transgenic mouse model. PLoS One 5, e11077.
11. Kashiwakura, J. C., Ando, T., Matsumoto, K., Kimura, M., Kitaura, J., Matho, M. H., Zajonc, D. M., Ozeki, T., Ra, C., MacDonald, S. M., et al. (2012). Histamine-releasing factor has a proinflammatory role in mouse models of asthma and allergy. J Clin Invest 122, 218-228.
12. Langdon, J. M., Schroeder, J. T., Vonakis, B. M., Bieneman, A. P., Chichester, K., and Macdonald, S. M. (2008). Histamine-releasing factor/translationally controlled tumor protein (HRF/TCTP)-induced histamine release is enhanced with SHIP-1 knockdown in cultured human mast cell and basophil models. J Leukoc Biol.
13. MacDonald, S. M. (1996). Histamine-releasing factors. Curr Opin Immunol 8, 778-783.
14. Macdonald, S. M. (2012). Potential role of histamine releasing factor (HRF) as a therapeutic target for treating asthma and allergy. J Asthma Allergy 5, 51-59.

### EXAMPLES

1. Synthesis—Experimental Procedure and Characterization of Products

1.1 General

All products described have been analyzed by conventional physical methods such as ¹H NMR, ¹³C NMR, infrared (IR), mass spectroscopy (MS). The ¹H and ¹³C NMR spectra were performed in deuterated chloroform CDCl₃, or deuterated methanol MeOD, using a Bruker 300 (or 400) spectrometer. The chemical displacements of the ¹H NMR spectra are reported in ppm according to an internal standard (TMS) or according to chloroform (7.26 ppm). The following abbreviations are used when describing the NMR spectra: (m) multiplet, s (singlet), d (doublet), t (triplet), dd (doublet of doublets), td (triplet of doublets), q (quadruplet), qui (quintuplet), sex (sextuplet). Chemical shifts in ¹³C NMR spectra are reported in ppm according to the central peak of chloroform (77.14 ppm). The IR spectra were made using a Bruker Vector 22 spectrophotometer and are reported in number of waves (cm⁻¹). The MS analyses were recorded by a Micromass spectrometer. TLC analyses were performed on Merck 60F silica supports. The silica used for the chromatographic column purifications corresponds to Merck gel 60 (0.015-0.040 mm). The melting points (mp) were performed on a Bichi B-450 apparatus and were not corrected. The high-resolution masses (HRMS) were made on a Brucker MicroTOF spectrometer, using methanol as solvent and ESI and APCI as ionization sources. The calculated and found values (m/z) are reported in daltons. Unless otherwise stated, the reagents used are commercial products that have been used without further purification beforehand. The same applies to the organic solvents used in the syntheses described in this document.

1.2 General Procedure 1

Introduce into a reaction tube the N-tosylhydrazone (1.0 mmol), the appropriate boronic acid (1.5 mmol), and K₂CO₃ (1.5 mmol). Dry dioxane (7 mL) is added and the mixture is first stirred at room temperature for 10 minutes under a stream of argon. In a second step, the tube is sealed, and the reaction medium is stirred at 110° C. for 12 hours (or until the end of the reaction which is followed by TLC). When the reaction is complete, the crude reaction product is cooled to room temperature and the solvent is evaporated under vacuum. Dichloromethane (DCM) and a saturated aqueous solution of NaHCO₃ are added and then the two phases are separated. Subsequently, the aqueous phase is extracted three times using DCM. The combined organic phases are then washed with a saturated aqueous solution of NaCl, then dried over MgSO₄, and finally filtered. The solvent is then removed under reduced pressure using a rotary evaporator. The products obtained are finally purified by silica or alumina chromatographic columns.

1.3 General Procedure No. 2

Place the precatalyst Xantphos-Pd-G3 (2 mol %), the thioglycoside (1.0 mmol) and the corresponding halide (1.0 mmol) in a round-bottomed flask. After purging the medium with argon, tetrahydrofuran (THF) is added (4 mL). While stirring at room temperature, triethylamine (NEt₃) (1.0 mmol) is added to the reaction medium. The mixture is stirred under argon at room temperature for 30 minutes. When the reaction is complete, the solvent is evaporated under reduced pressure and the reaction crude is purified by silica chromatographic column to deliver the expected product.

1.4 General Procedure No. 3

Place N-tosylhydrazone (1.0 mmol), t-BuLi (2.2 mmol), Pd₂dba₃ (10 mol %), XPhos (20 mol %) and the corresponding aryl iodide (1.1 mmol) in a reaction tube. Dry dioxane (5 mL) is added, the tube is sealed and the whole is stirred at 90° C. for 8 hours. When the reaction is complete, and cooled to room temperature, dichloromethane (DCM) is added to the reaction medium. The crude is then filtered through a celite block and the solvent is evaporated under reduced pressure. The last step consists of purification on a silica chromatographic column.

1.5 General Procedure No. 4

The first step is the dissolution of the appropriate carboxylic acid (1.0 mmol), HOBt (1.2 mmol), in N,N-dimethylformamide (DMF) (10 mL). The mixture is stirred for 15 minutes at room temperature under argon atmosphere. Then 8-aminoquinoline (1.2 mmol) is added and the reaction medium is stirred at room temperature overnight. The resulting crude reaction product is then extracted three times with a saturated aqueous solution of NH₄Cl. The combined organic phases are washed with a saturated aqueous solution of NaCl, then with water, before being dried over MgSO₄ and filtered. Once the solvent has been evaporated by rotary evaporator, the products obtained are purified by silica chromatography column before being fed to the next stage.

In a second step, introduce into a dry tube Cu(OAc)₂.H₂O (20 mol %), Ag₂CO₃ (2.0 mmol), benzamide (1.0 mmol) and thiosugar (2.0 mmol). The medium is then purged with argon for 10 minutes before the addition of dimethylsulfoxide (DMSO) (10 mL). Seal the tube and agitate at 110° C. for 12 hours. After completion of the reaction, the medium is cooled to room temperature before ice is added. Then the crude reaction product is extracted three times with ethyl acetate (EtOAc). Finally, the organic phases are dried over MgSO₄, filtered, evaporated and purified by silica column chromatography.

1.6 General Procedure No. 5

Place in a dry tube Co(acac)₂ (10 mol %), Ag₂CO₃ (0.53 mmol), the corresponding commercial carboxylic acid (0.19 mmol) and thiosugar (0.29 mmol). The medium is then purged with argon for 10 minutes before the addition of trifluorotoluene (2 mL). Seal the tube and agitate at 150° C. until the reaction is complete. Once the medium has cooled to room temperature, the reaction crude is filtered through a small celite block and rinsed three times with ethyl acetate (EtOAc). After evaporation of the solvent under reduced pressure, the crude is then purified by chromatography using a silica column.

1.7 Characteristic Data of the Different Tetrahydronaphthalene Type Derivatives

### 1-phenyl-1,2,3,4-tetrahydronaphthalene [RA002]

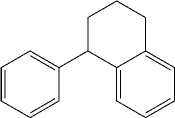 [SMILES: c1ccc(C2CCCc3ccccc32)cc1]

Transparent oil obtained according to general procedure 1 (31.8 mg, 48% yield); TLC R_{f}=0.50 (Cyclohexane, SiO₂); IR (film, cm⁻¹) 2924, 2853, 1672, 1599, 1491, 1448, 1158, 1079, 1033, 1003; ¹H NMR (300 MHz, CDCl₃) δ (ppm) 7.32-7.25 (m, 2H), 7.23-7.17 (m, 1H), 7.16-7.11 (m, 3H), 7.09 (d, J=3.3 Hz, 1H), 7.03 (ddd, J=8.7, 6.1, 2.7 Hz, 1H), 6.85 (d, J=8.0 Hz, 1H), 4.13 (t, J=6.6 Hz, 1H), 2.98-2.80 (m, 2H), 2.24-2.11 (m, 1H), 1.90 (qdd, J=10.0, 5.0, 2.0 Hz, 2H), 1.81-1.66 (m, 1H); ¹³C NMR (75 MHz, CDCl₃) δ (ppm) 147.7 (C_{q}), 139.5 (C_{q}), 137.7 (C_{q}), 130.3 (CH), 129.1 (CH), 129.0 (2×CH), 128.4 (2×CH), 126.1 (CH), 126.0 (CH), 125.8 (CH), 45.8 (CH), 33.4 (CH₂), 29.9 (CH₂), 21.1 (CH₂).

### 1-(4-methoxyphenyl)-1,2,3,4-tetrahydronaphthalene [RA004]

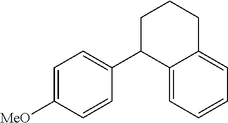 [SMILES: COc1ccc(C2CCCc3ccccc32)cc1]

Yellowish transparent oil obtained according to general procedure 1 (40.1 mg, 53% yield); TLC R_{f}=0.92 (Cyclohexane/EtOAc, 7:3, SiO₂); IR (film, cm⁻¹) 2925, 2854, 1611, 1583, 1511, 1463, 1448, 1302, 1243, 1177, 1109, 1038; ¹H NMR (300 MHz, CDCl₃) δ (ppm) 7.16-7.08 (m, 2H), 7.06-6.98 (m, 3H), 6.88-6.79 (m, 3H), 4.07 (t, J=6.5 Hz, 1H), 3.79 (s, 3H), 2.97-2.77 (m, 2H), 2.21-2.07 (m, 1H), 1.88 (tdd, J=11.6, 4.8, 1.9 Hz, 2H), 1.78-1.71 (m, 1H); ¹³C NMR (75 MHz, CDCl₃) δ (ppm) 145.5 (C_{q}), 139.9 (C_{q}), 139.8 (C_{q}), 137.7 (C_{q}), 130.3 (CH), 129.8 (2×CH), 129.1 (CH), 126.0 (CH), 125.7 (CH), 113.8 (2×CH), 55.4 (CH₃), 44.9 (CH), 33.5 (CH₂), 29.9 (CH₂), 21.1 (CH₂).

### 1-(4-chlorophenyl)-1,2,3,4-tetrahydronaphthalene [RA005]

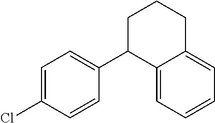 [SMILES: Clc1ccc(C2CCCc3ccccc32)cc1]

Amorphous solid obtained according to general procedure 1 (40.2 mg, 52% yield); TLC R_{f}=0.84 (Cyclohexane/EtOAc, 9:1, SiO₂); IR (film, cm⁻¹) 2926, 1672, 1595, 1489, 1452, 1400, 1092, 1014; ¹H NMR (300 MHz, CDCl₃) δ (ppm) 7.29-7.21 (m, 2H), 7.13 (dd, J=5.0, 1.0 Hz, 2H), 7.09-6.98 (m, 3H), 6.81 (d, J=7.7 Hz, 1H), 4.10 (t, J=6.4 Hz, 1H), 2.97-2.78 (m, 2H), 2.16 (qd, J=9.8, 5.1 Hz, 1H), 1.93-1.80 (m, 2H), 1.79-1.70 (m, 1H); ¹³C NMR (75 MHz, CDCl₃) δ (ppm) 146.2 (C_{q}), 138.9 (C_{q}), 137.7 (C_{q}), 131.8 (C_{q}), 130.3 (2×CH), 130.2 (CH), 129.2 (CH), 128.5 (2×CH), 126.3 (CH), 125.9 (CH), 45.2 (CH), 33.4 (CH₂), 29.8 (CH₂), 21.0 (CH₂).

### 1-(3,4,5-trimethoxyphenyl)-1,2,3,4-tetrahydronaphthalene [AX013]

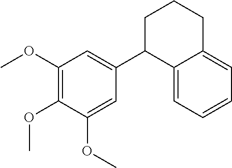 [SMILES: COc1cc(C2CCCc3ccccc32)cc(OC)c1OC]

Yellowish transparent oil obtained according to general procedure 1 (50.6 mg, 54% yield); TLC R_{f}=0.50 (Cyclohexane/EtOAc, 8:2, SiO₂); IR (film, cm⁻¹) 2927, 1588, 1508, 1449, 1418, 1329, 1232, 1124, 1011; ¹H NMR (300 MHz, CDCl₃) δ (ppm) 7.13 (dd, J=4.9, 1.1 Hz, 2H), 7.10-7.02 (m, 1H), 6.89 (d, J=7.7 Hz, 1H), 6.33 (s, 2H), 4.10-4.00 (m, 1H), 3.85 (s, 3H), 3.79 (s, 6H), 3.02-2.80 (m, 2H), 2.25-2.11 (m, 1H), 2.01-1.86 (m, 2H), 1.83-1.73 (m, 1H); ¹³C NMR (75 MHz, CDCl₃) δ (ppm) 153.1 (2×C_{q}), 143.2 (C_{q}), 139.3 (C_{q}), 137.5 (C_{q}), 136.3 (C_{q}), 130.1 (CH), 129.0 (CH), 126.1 (CH), 125.7 (CH), 106.0 (2×CH), 60.9 (CH), 56.2 (2×CH₃), 46.3 (CH₃), 33.4 (CH₂), 29.9 (CH₂), 21.5 (CH₂); HRMS (APCI) (M+Na)⁺ m/z calculated for C₁₉H₂₂O₃Na 321.1467, found 321.1470.

### 1,2,3,4-tetrahydro-1,2′-binaphthalene [AC014]

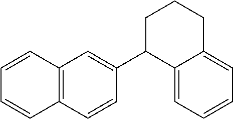 [SMILES: c1ccc2c(c1)CCCC2c1ccc2ccccc2c1]

White solid obtained according to general procedure 1 (39.0 mg, 47% yield); mp: 72.7-73.4° C.; TLC R_{f}=0.85 (Cyclohexane/EtOAc, 7:3, SiO₂); IR (film, cm⁻¹) 2927, 2854, 1589, 1507, 1491, 1449, 1127; ¹H NMR (300 MHz, CDCl₃) δ (ppm) 7.85-7.72 (m, 3H), 7.54 (s, 1H), 7.47-7.40 (m, 2H), 7.25 (d, J=6.7 Hz, 1H), 7.15 (q, J=7.2 Hz, 2H), 7.02 (t, J=7.4 Hz, 1H), 6.86 (d, J=7.8 Hz, 1H), 4.28 (t, J=5.6 Hz, 1H), 3.03-2.82 (m, 2H), 2.28-2.16 (m, 1H), 2.05-1.89 (m, 2H), 1.88-1.75 (m, 1H); ¹³C NMR (75 MHz, CDCl₃) δ (ppm) 145.0 (C_{q}), 139.4 (C_{q}), 137.8 (C_{q}), 133.6 (C_{q}), 132.2 (C_{q}), 130.4 (CH), 129.2 (CH), 128.1 (CH), 127.7 (2×CH), 127.5 (CH), 127.4 (CH), 126.1 (CH), 126.0 (CH), 125.8 (CH), 125.4 (CH), 46.0 (CH), 33.3 (CH₂), 30.0 (CH₂), 21.3 (CH₂).

### 1-(4-bromophenyl)-1,2,3,4-tetrahydronaphthalene [AC023]

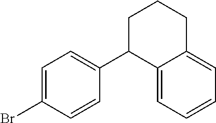 [SMILES: Brc1ccc(C2CCCc3ccccc32)cc1]

Transparent oil obtained according to general procedure 1 (30.0 mg, 33% yield); TLC R_{f}=0.81 (Cyclohexane/EtOAc, 7:3, SiO₂); IR (film, cm⁻¹) 3017, 2929, 2856, 1486, 1449, 1403, 1073, 1010; ¹H NMR (300 MHz, CDCl₃) δ (ppm) 7.42 (dt, J=8.0, 2.4 Hz, 2H), 7.16 (dt, J=5.1, 2.5 Hz, 2H), 7.10-7.02 (m, 1H), 6.99 (dt, J=8.0, 3.0 Hz, 2H), 6.83 (d, J=7.1 Hz, 1H), 4.11 (t, J=6.4 Hz, 1H), 3.00-2.81 (m, 2H), 2.25-2.09 (m, 1H), 1.94-1.72 (m, 3H); ¹³C NMR (75 MHz, CDCl₃) δ (ppm) 146.7 (C_{q}), 138.8 (C_{q}), 137.7 (C_{q}), 131.4 (2×CH), 130.7 (2×CH), 130.2 (CH), 129.2 (CH), 126.3 (CH), 125.9 (CH), 119.9 (C_{q}), 45.2 (CH), 33.3 (CH₂), 29.8 (CH₂), 20.9 (CH₂).

### Isopropyl 4-(1,2,3,4-tetrahydronaphthalen-1-yl)benzoate [AC034]

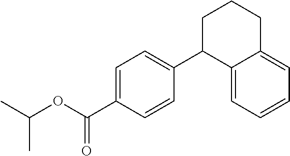 [SMILES: CC(C)OC(=O)c1ccc(C2CCCc3ccccc32)cc1]

Pale yellow oil obtained according to general procedure 1 (40.0 mg, 47% yield); TLC R_{f}=0.78 (Cyclohexane/EtOAc, 7:3, SiO₂); IR (film, cm⁻¹) 2979, 2931, 2856, 1713, 1610, 1451, 1415, 1373, 1352, 1274, 1178, 1098, 1019; ¹H NMR (300 MHz, CDCl₃) δ (ppm) 7.97 (d, J=8.1 Hz, 2H), 7.23-7.09 (m, 4H), 7.04 (dd, J=11.0, 5.7 Hz, 1H), 6.80 (d, J=7.6 Hz, 1H), 5.26 (dt, J=12.7, 6.4 Hz, 1H), 4.20 (t, J=6.6 Hz, 1H), 2.97-2.76 (m, 2H), 2.18 (dd, J=12.9, 6.3 Hz, 1H), 1.92-1.76 (m, 3H), 1.37 (d, J=6.2 Hz, 6H); ¹³C NMR (75 MHz, CDCl₃) δ (ppm) 166.3 (C_{q}), 152.9 (C_{q}), 145.6 (C_{q}), 138.7 (C_{q}), 137.7 (C_{q}), 130.2 (CH), 129.7 (2×CH), 129.2 (CH), 128.9 (2×CH), 126.3 (CH), 125.9 (CH), 68.3 (CH), 45.8 (CH), 33.3 (CH₂), 29.8 (CH₂), 22.1 (2×CH₃), 21.1 (CH₂); HRMS (APCI) (M+H)⁺ m/z calculated for C₂₀H₂₃O₂ 295.1693, found 295.1700.

### 1-(3,5-dimethylphenyl)-1,2,3,4-tetrahydronaphthalene [AC035]

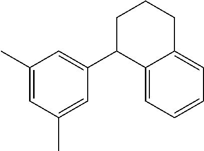 [SMILES: Cc1cc(C)cc(C2CCCc3ccccc32)c1]

White solid obtained according to general procedure 1 (47.0 mg, 62% yield); mp: 57.6-58.6° C.; TLC R_{f}=0.82 (Cyclohexane/EtOAc, 7:3, SiO₂); IR (film, cm⁻¹) 2932, 2856, 2361, 1706, 1602, 1437, 1179, 1120; ¹H NMR (300 MHz, CDCl₃) δ (ppm) 7.19-7.13 (m, 2H), 7.07 (ddd, J=8.6, 5.9, 3.0 Hz, 1H), 6.89 (dd, J=3.4, 2.7 Hz, 2H), 6.77 (s, 2H), 4.07 (t, J=6.1 Hz, 1H), 3.00-2.81 (m, 2H), 2.31 (d, J=0.5 Hz, 6H), 2.24-2.12 (m, 1H), 2.02-1.87 (m, 2H), 1.86-1.74 (m, 1H); ¹³C NMR (75 MHz, CDCl₃) δ (ppm) 147.6 (C_{q}), 139.8 (C_{q}), 137.8 (2×C_{q}), 137.6 (C_{q}), 130.3 (CH), 129.0 (CH), 127.8 (CH), 126.8 (2×CH), 125.9 (CH), 125.7 (CH), 45.8 (CH₃), 33.5 (CH₂), 30.0 (CH₂), 21.5 (CH₃), 21.4 5 (CH₂).

### 1-(3-vinylphenyl)-1,2,3,4-tetrahydronaphthalene [AC037]

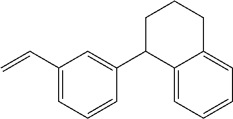 [SMILES: C=Cc1cccc(C2CCCc3ccccc32)c1]

Transparent oil obtained according to general procedure 1 (57.6 mg, 77% yield); TLC R_{f}=0.79 (Cyclohexane/EtOAc, 7:3, SiO₂); IR (film, cm⁻¹) 3016, 2930, 2856, 1631, 1600, 1578, 1491, 1451, 1403; ¹H NMR (300 MHz, CDCl₃) δ (ppm) 7.30 (dd, J=8.3, 2.4 Hz, 2H), 7.16 (dd, J=8.5, 5.5 Hz, 3H), 7.09-6.97 (m, 2H), 6.87 (d, J=7.6 Hz, 1H), 6.71 (dd, J=17.6, 10.8 Hz, 1H), 5.74 (d, J=17.6 Hz, 1H), 5.24 (d, J=10.9 Hz, 1H), 4.14 (t, J=6.0 Hz, 1H), 2.99-2.85 (m, 2H), 2.20 (ddd, J=11.8, 8.4, 5.6 Hz, 1H), 2.02-1.71 (m, 3H); ¹³C NMR (75 MHz, CDCl₃) δ (ppm) 147.9 (C_{q}), 139.4 (C_{q}), 137.7 (C_{q}), 137.6 (C_{q}), 137.1 (CH), 130.3 (CH), 129.1 (CH), 128.6 (2×CH), 127.0 (CH), 126.1 (CH), 125.8 (CH), 123.9 (CH), 113.8 (CH₂), 45.8 (CH), 33.4 (CH₂), 29.9 (CH₂), 21.3 (CH₂).

### 4-(1,2,3,4-tetrahydronaphthalen-1-yl)benzaldehyde [AC038]

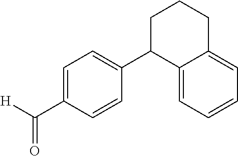 [SMILES: O=Cc1ccc(C2CCCc3ccccc32)cc1]

Opaque oil obtained according to general procedure 1 (25.0 mg, 33% yield); TLC R_{f}=0.71 (Cyclohexane/EtOAc, 7:3, SiO₂); IR (film, cm⁻¹) 2929, 2857, 1701, 1605, 1574, 1491, 1450, 1306, 1211, 1168; ¹H NMR (300 MHz, CDCl₃) δ (ppm) 9.99 (s, 1H), 7.83-7.77 (m, 2H), 7.27 (d, J=7.8 Hz, 2H), 7.18-7.13 (m, 2H), 7.05 (ddd, J=8.6, 5.6, 3.4 Hz, 1H), 6.79 (d, J=7.9 Hz, 1H), 4.23 (t, J=6.4 Hz, 1H), 2.98-2.82 (m, 2H), 2.27-2.14 (m, 1H), 1.96-1.76 (m, 3H); ¹³C NMR (75 MHz, CDCl₃) δ (ppm) 192.13 (CH), 155.07 (C_{q}), 138.26 (C_{q}), 137.73 (C_{q}), 134.72 (C_{q}), 130.19 (CH), 129.99 (2×CH), 129.63 (2×CH), 129.34 (CH), 126.46 (CH), 125.99 (CH), 45.97 (CH), 33.19 (CH₂), 29.77 (CH₂), 20.93 (CH₂); HRMS (APCI) (M+H)⁺ m/z calculated for C₁₇H₁₇O 237.1274, found 237.1272.

### 1-(3-iodophenyl)-1,2,3,4-tetrahydronaphthalene [AC041]

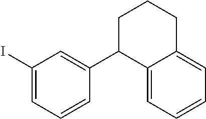 [SMILES: Ic1cccc(C2CCCc3ccccc32)c1]

Transparent oil obtained according to general procedure 1 (48.6 mg, 44% yield); TLC R_{f}=0.82 (Cyclohexane/EtOAc, 7:3, SiO₂); IR (film, cm⁻¹) 3058, 3016, 2931, 2855, 1674, 1587, 1562, 1491, 1470, 1451, 1417; ¹H NMR (300 MHz, CDCl₃) δ (ppm) 7.55 (dt, J=6.8, 1.9 Hz, 1H), 7.50 (d, J=1.7 Hz, 1H), 7.15 (dd, J=4.8, 1.0 Hz, 2H), 7.09-6.98 (m, 3H), 6.83 (d, J=7.7 Hz, 1H), 4.07 (t, J=6.5 Hz, 1H), 2.97-2.80 (m, 2H), 2.23-2.11 (m, 1H), 1.94-1.70 (m, 3H); ¹³C NMR (75 MHz, CDCl₃) δ (ppm) 150.1 (C_{q}), 138.6 (C_{q}), 137.8 (CH), 137.7 (C_{q}), 135.2 (CH), 130.1 (2×CH), 129.2 (CH), 128.3 (CH), 126.3 (CH), 125.9 (CH), 94.6 (CH), 45.4 (CH), 33.3 (CH₂), 29.8 (CH₂), 21.0 (CH₂); HRMS (ESI) (M+Na)⁺ m/z calculated for C₁₆H₁₅INa 357.0111, found 357.0121.

### 1-(4-(benzyloxy)-3-chlorophenyl)-1,2,3,4-tetrahydronaphthalene [AC042]

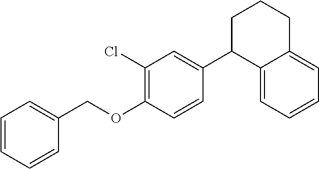 [SMILES: Clc1cc(C2CCCc3ccccc32)ccc1OCc1ccccc1]

Pale yellow solid obtained according to general procedure 1 (50.0 mg, 45% yield); mp: 89.9-91.5° C.; TLC R_{f}=0.80 (Cyclohexane/EtOAc, 7:3, SiO₂); IR (film, cm⁻¹) 2931, 2857, 1603, 1497, 1452, 1381, 1286, 1251, 1061, 1024; ¹H NMR (300 MHz, CDCl₃) δ (ppm) 7.49 (dd, J=8.0, 1.6 Hz, 2H), 7.44-7.32 (m, 3H), 7.15 (d, J=3.9 Hz, 3H), 7.06 (dt, J=8.7, 4.2 Hz, 1H), 6.90 (d, J=1.6 Hz, 2H), 6.85 (d, J=7.7 Hz, 1H), 5.14 (s, 2H), 4.06 (t, J=6.3 Hz, 1H), 2.98-2.79 (m, 2H), 2.21-2.04 (m, 1H), 1.94-1.70 (m, 3H); ¹³C NMR (75 MHz, CDCl₃) δ (ppm) 152.5 (C_{q}), 141.3 (C_{q}), 139.0 (C_{q}), 137.6 (C_{q}), 136.9 (C_{q}), 130.6 (CH), 130.2 (CH), 129.2 (CH), 128.7 (2×CH), 128.0 (CH), 128.0 (CH), 127.2 (2×CH), 126.2 (CH), 125.9 (CH), 123.1 (C_{q}), 114.0 (CH), 71.0 (CH₂), 44.7 (CH), 33.3 (CH₂), 29.8 (CH₂), 20.9 (CH₂); HRMS (ESI) (M+Na)⁺ m/z calculated for C₂₃H₂₁ClONa 371.1179, found 371.1181.

### 1-(3-methoxyphenyl)-1,2,3,4-tetrahydronaphthalene [AC046]

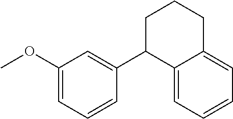 [SMILES: COc1cccc(C2CCCc3ccccc32)c1]

Transparent oil obtained according to general procedure 1 (60.8 mg, 80% yield); TLC R_{f}=0.76 (Cyclohexane/EtOAc, 7:3, SiO₂); IR (film, cm⁻¹) 2933, 2858, 2834, 2360, 2341, 1610, 1582, 1489, 1452, 1281, 1262, 1223; ¹H NMR (300 MHz, CDCl₃) δ (ppm) 7.25 (dd, J=9.5, 6.3 Hz, 1H), 7.20-7.15 (m, 2H), 7.13-7.05 (m, 1H), 6.92 (d, J=7.9 Hz, 1H), 6.80 (ddd, J=8.2, 2.6, 0.9 Hz, 1H), 6.75 (d, J=7.6 Hz, 1H), 6.73-6.70 (m, 1H), 4.18-4.11 (m, 1H), 3.81 (s, 3H), 3.04-2.81 (m, 2H), 2.26-2.14 (m, 1H), 2.02-1.89 (m, 2H), 1.87-1.75 (m, 1H); ¹³C NMR (75 MHz, CDCl₃) δ (ppm) 159.6 (C_{q}), 149.3 (C_{q}), 139.3 (C_{q}), 137.6 (C_{q}), 130.3 (CH), 129.3 (CH), 129.1 (CH), 126.0 (CH), 125.8 (CH), 121.5 (CH), 115.1 (CH), 111.1 (CH), 55.3 (CH), 45.8 (CH₃), 33.2 (CH₂), 29.9 (CH₂), 21.1 (CH₂); HRMS (APCI) (M+H)⁺ m/z calculated for C₁₇H₁₉O 239.1430, found 239.1464.

### N-(4-(4-methoxyphenyl)-1,2,3,4-tetrahydronaphthalen-1-yl)acetamide [AC050]

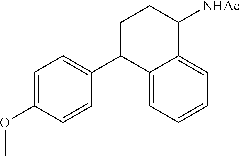 [SMILES: COc1ccc(C2CCC(NC(C)=O)c3ccccc32)cc1]

Transparent amorphous solid obtained according to general procedure 1 (34.8 mg, 55% yield, d.e=72%); mixture of diastereoisomers [cis (min): 14%, trans (maj): 86%]; TLC R_{f}=0.06 (Cyclohexane/EtOAc, 7:3, SiO₂); IR (film, cm⁻¹) 2932, 2857, 2835, 1635, 1542, 1511, 1445, 1243, 1178, 1112, 1036; ¹H NMR (300 MHz, CDCl₃) δ (ppm) major dia (trans): 7.33 (d, J=7.4 Hz, 1H), 7.23-7.08 (m, 2H), 7.04-6.98 (m, 2H), 6.88 (d, J=7.6 Hz, 1H), 6.86-6.77 (m, 2H), 5.92 (d, J=8.2 Hz, 1H), 5.21 (dt, J=8.2, 5.4 Hz, 1H), 4.03 (t, J=6.3 Hz, 1H), 3.79 (s, 3H), 2.22-2.09 (m, 1H), 2.04 (s, 3H), 2.00-1.81 (m, 3H); minor dia (cis): 7.33 (d, J=7.4 Hz, 1H), 7.23-7.08 (m, 2H), 6.97-6.92 (m, 2H), 6.88 (d, J=7.6 Hz, 1H), 6.86-6.77 (m, 2H), 5.85 (d, J=8.8 Hz, 1H), 5.31 (dd, J=12.7, 7.7 Hz, 1H), 4.17-4.07 (m, 1H), 3.78 (s, 3H), 2.22-2.09 (m, 1H), 2.05 (s, 3H), 2.00-1.81 (m, 3H); ¹³C NMR (75 MHz, CDCl₃) δ (ppm) major dia (trans): 169.4 (C_{q}), 158.1 (C_{q}), 140.3 (C_{q}), 138.6 (C_{q}), 137.2 (C_{q}), 130.3 (CH), 129.7 (2×CH), 128.9 (CH), 127.6 (CH), 126.8 (CH), 113.9 (2×CH), 55.4 (CH₃), 47.8 (CH), 44.6 (CH), 29.7 (CH₂), 27.6 (CH₂), 23.7 (CH₃); minor dia (cis): 169.4 (C_{q}), 158.1 (C_{q}), 140.3 (C_{q}), 138.6 (C_{q}), 137.2 (C_{q}), 130.3 (CH), 129.7 (2×CH), 128.9 (CH), 128.0 (CH), 127.5 (CH), 113.9 (2×CH), 55.4 (CH₃), 48.0 (CH), 44.7 (CH), 30.6 (CH₂), 28.5 (CH₂), 23.7 (CH₃); HRMS (ESI) (M+Na)⁺ m/z calculated for C₁₉H₂₁NO₂Na 318.1470, found 318.1466.

### N-(4-(3,4,5-trimethoxyphenyl)-1,2,3,4-tetrahydronaphthalen-1-yl)acetamide [AC051]

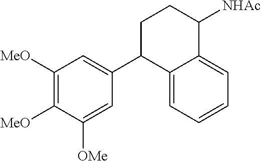 [SMILES: COc1cc(C2CCC(NC(C)=O)c3ccccc32)cc(OC)c1OC]

Transparent amorphous solid obtained according to general procedure 1 (26.3 mg, 28% yield, d.e=80%); crude reaction product initially composed of a mixture of diastereoisomers [cis (min): 47%, trans (maj): 53%, d.e: 6%] enriched by recrystallization with a cyclohexane/diisopropanol mixture [cis (initially min): 90%, trans (initially maj)]: 10%, d.e: 80%]; TLC R_{f}=0.12 (Cyclohexane/EtOAc, 6:4, SiO₂); IR (film, cm⁻¹) 3277, 2937, 1648, 1539, 1421, 1330, 1235, 1126; ¹H NMR (300 MHz, CDCl₃) δ (ppm) major dia (cis): 7.35 (d, J=6.7 Hz, 1H), 7.25-7.11 (m, 2H), 6.93 (d, J=7.3 Hz, 1H), 6.31 (s, 2H), 5.78-5.73 (m, 1H), 5.22 (ddd, J=5.3, 4.7, 2.7 Hz, 1H), 4.01-3.95 (m, 1H), 3.85 (s, 3H), 3.80 (s, 6H), 2.20-2.10 (m, 2H), 2.06 (s, 3H), 2.05-1.96 (m, 1H), 1.94-1.85 (m, 1H); minor dia (trans): 7.35 (d, J=6.7 Hz, 1H), 7.25-7.11 (m, 2H), 6.93 (d, J=7.3 Hz, 1H), 6.26 (s, 2H), 5.85-5.81 (m, 1H), 5.18 (ddd, J=5.2, 2.6, 1.2 Hz, 1H), 4.01-3.95 (m, 1H), 3.84 (s, 3H), 3.77 (s, 6H), 2.20-2.10 (m, 1H), 2.06 (s, 3H), 2.05-1.96 (m, 2H), 1.94-1.85 (m, 1H); ¹³C NMR (75 MHz, CDCl₃) δ (ppm) major dia (cis): 169.4 (C_{q}), 153.3 (2×C_{q}), 142.0 (C_{q}), 139.9 (C_{q}), 137.1 (C_{q}), 136.8 (C_{q}), 130.1 (CH), 129.0 (CH), 127.7 (CH), 127.0 (CH), 106.3 (2×CH), 61.0 (CH), 56.4 (2×CH₃), 47.7 (CH), 46.0 (CH₃), 29.5 (CH₂), 28.1 (CH₂), 23.7 (CH₃); minor dia trans): 169.4 (C_{q}), 153.3 (2×C_{q}), 142.0 (C_{q}), 139.9 (C_{q}), 137.1 (C_{q}), 136.8 (C_{q}), 130.3 (CH), 129.0 (CH), 127.5 (CH), 127.0 (CH), 105.9 (2×CH), 61.0 (CH), 56.2 (2×CH₃), 48.1 (CH), 46.2 (CH₃), 29.5 (CH₂), 28.1 (CH₂), 23.7 (CH₃); HRMS (ESI) (M+Na)⁺ m/z calculated for C₂₁H₂₅NO₄Na 378.1681, found 378.1675.

### Cis-isopropyl 4-(4-acetamido-1,2,3,4-tetrahydronaphthalen-1-yl)benzoate [AC056-cis]

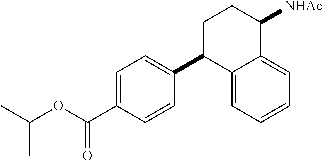

White solid obtained according to general procedure 1 (15.0 mg, 18% yield); Purified by HPLC using an Xbridge C18 type column (4.6×150 mm, 5 μm) and a H₂O/MeOH mixture (30:70) as solvent; mp: 146.8-148.4° C.; TLC R_{f}=0.50 (EtOAc, SiO₂); IR (film, cm⁻¹) 2959, 2923, 2852, 1701, 1649, 1605, 1450; ¹H NMR (300 MHz, CDCl₃) δ (ppm) 7.95 (d, J=8.2 Hz, 2H), 7.35 (d, J=7.9 Hz, 1H), 7.22 (t, J=7.5 Hz, 1H), 7.11 (d, J=8.2 Hz, 2H), 6.80 (d, J=7.6 Hz, 1H), 5.75 (d, J=8.1 Hz, 1H), 5.27 (ddd, J=18.6, 12.6, 7.2 Hz, 2H), 4.18 (d, J=6.2 Hz, 1H), 2.30-2.15 (m, 2H), 2.06 (s, 3H), 1.95-1.83 (m, 1H), 1.79-1.70 (m, 1H), 1.64 (s, 1H), 1.35 (d, J=6.2 Hz, 6H); ¹³C NMR (75 MHz, CDCl₃) δ (ppm) 169.6 (C_{q}), 166.1 (C_{q}), 151.7 (C_{q}), 139.3 (C_{q}), 137.6 (C_{q}), 130.3 (CH), 129.9 (2×CH), 129.3 (C_{q}), 128.8 (2×CH), 128.1 (CH), 127.7 (CH), 127.1 (CH), 68.4 (CH), 48.0 (CH), 45.7 (CH), 30.5 (CH₂), 28.5 (CH₂), 23.7 (CH₃), 22.1 (2×CH₃); HRMS (ESI) (M+Na)⁺ m/z calculated for C₂₂H₂₅NO₃Na 374.1727, found 374.1740.

### 4-(naphthalen-2-yl)chroman [AC069]

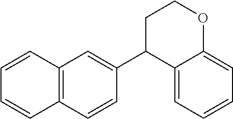 [SMILES: c1ccc2c(c1)OCCC2c1ccc2ccccc2c1]

Beige solid obtained according to general procedure 1 (22.3 mg, 27% yield); mp: mp: 84.8-85.6° C.; TLC R_{f}=0.74 (Cyclohexane/EtOAc, 7:3, SiO₂); IR (film, cm⁻¹) 3054, 2950, 2878, 1604, 1581, 1487, 1452, 1308, 1269, 1248, 1222; ¹H NMR (300 MHz, CDCl₃) δ (ppm) 7.90-7.76 (m, 3H), 7.60 (s, 1H), 7.54-7.45 (m, 2H), 7.33 (dd, J=8.5, 1.7 Hz, 1H), 7.21 (tdd, J=7.0, 1.8, 0.6 Hz, 1H), 6.97 (dd, J=8.1, 0.8 Hz, 1H), 6.87 (tdd, J=8.7, 7.8, 1.2 Hz, 2H), 4.38 (t, J=6.5 Hz, 1H), 4.31-4.21 (m, 2H), 2.46-2.33 (m, 1H), 2.24 (dtd, J=18.1, 6.7, 4.1 Hz, 1H); ¹³C NMR (75 MHz, CDCl₃) δ (ppm) 155.34 (C_{q}), 143.07 (C_{q}), 133.48 (C_{q}), 132.41 (C_{q}), 130.86 (CH), 128.41 (CH), 128.06 (CH), 127.80 (CH), 127.72 (CH), 127.67 (CH), 126.78 (CH), 126.25 (CH), 125.75 (CH), 124.58 (C_{q}), 120.51 (CH), 116.96 (CH), 64.09 (CH₂), 41.37 (CH), 31.63 (CH₂); HRMS (APCI) (M+H)⁺ m/z calculated for C₁₉H₁₇O 261.1274, found 261.1273.

### N-(1,2,3,4-tetrahydro-[1,2′-binaphthalen]-4-yl) acetamide [AC070]

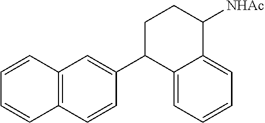 [SMILES: CC(=O)NC1CCC(c2ccc3ccccc3c2)c2ccccc21]

Transparent solid obtained according to general procedure 1 (39.0 mg, 48% yield, d.e=74%) mixture of diastereoisomers [cis (min): 13%, trans (maj): 87%]; TLC R_{f}=0.56 (EtOAc, SiO₂); IR (film, cm⁻¹) 3268, 3055, 2930, 2855, 1634, 1540, 1450, 1371; ¹H NMR (300 MHz, CDCl₃) δ (ppm) 7.79 (td, J=9.7, 5.3 Hz, 3H), 7.53 (s, 1H), 7.49-7.43 (m, 2H), 7.39 (d, J=7.4 Hz, 1H), 7.26 (d, J=1.9 Hz, 1H), 7.23 (d, J=7.0 Hz, 1H), 7.14 (dd, J=10.7, 4.3 Hz, 1H), 6.91 (d, J=7.7 Hz, 1H), 5.88 (d, J=7.8 Hz, 1H), 5.29-5.19 (m, 1H), 4.24 (t, J=6.1 Hz, 1H), 2.19 (dd, J=11.7, 6.1 Hz, 1H), 2.07 (s, 3H), 2.03 (dd, J=6.8, 4.2 Hz, 3H); ¹³C NMR (75 MHz, CDCl³) δ (ppm) 169.4 (C_{q}), 143.9 (C_{q}), 139.9 (C_{q}), 137.3 (C_{q}), 133.5 (C_{q}), 132.3 (C_{q}), 130.5 (CH), 129.1 (CH), 127.8 (2×CH), 127.7 (CH), 127.6 (CH), 127.0 (2×CH), 126.3 (CH), 125.7 (CH), 47.9 (CH), 45.6 (CH), 29.4 (CH₂), 27.8 (CH₂), 23.8 (CH₃); HRMS (APCI) (M+H)⁺ m/z calculated for C₂₂H₂₂NO 316.1696, found 316.1699.

### Cis-N-(1,2,3,4-tetrahydro-[1,2′-binaphthalen]-4-yl)acetamide [AC070-cis]

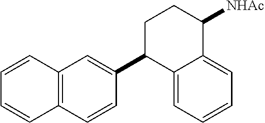

White solid obtained according to general procedure 1 (10.0 mg, 3% yield); Purified by HPLC using an Xbridge C18 type column (4.6×150 mm, 5 μm) and a H₂O/MeOH mixture (25:75) as solvent; mp: 175.3-176.2° C.; TLC R_{f}=0.67 (EtOAc, SiO₂); IR (film, cm⁻¹) 3268, 3055, 2930, 2855, 1634, 1540, 1450, 1371; ¹H NMR (300 MHz, CDCl₃) δ (ppm) 7.79 (ddd, J=14.5, 7.4, 5.7 Hz, 3H), 7.50 (s, 1H), 7.48-7.43 (m, 2H), 7.39 (d, J=7.4 Hz, 1H), 7.26-7.17 (m, 2H), 7.12 (t, J=7.2 Hz, 1H), 6.89 (d, J=7.5 Hz, 1H), 5.73 (d, J=9.0 Hz, 1H), 5.40 (dd, J=13.1, 8.9 Hz, 1H), 4.36-4.27 (m, 1H), 2.33-2.22 (m, 2H), 2.10 (s, 3H), 2.02 (dd, J=17.5, 8.3 Hz, 1H), 1.77 (dd, J=18.9, 9.6 Hz, 1H); ¹³C NMR (75 MHz, CDCl₃) δ (ppm) 170.5 (C_{q}), 143.9 (C_{q}), 143.3 (C_{q}), 140.0 (C_{q}), 137.7 (C_{q}), 133.6 (C_{q}), 130.6 (CH), 128.4 (CH), 128.0 (CH), 127.8 (2×CH), 127.6 (CH), 127.5 (CH), 127.1 (CH), 127.0 (CH), 126.2 (CH), 125.7 (CH), 48.2 (CH), 45.9 (CH), 30.5 (CH₂), 28.8 (CH₂), 23.8 (CH₃); HRMS (APCI) (M+H)⁺ m/z calculated for C₂₂H₂₂NO 316.1696, found 316.1699.

### Cis-N-(4-(1H-indol-5-yl)-1,2,3,4-tetrahydronaphthalen-yl)acetamide [AC081-cis]

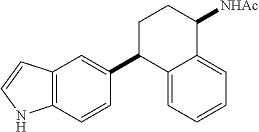

White solid obtained according to general procedure 1 (17.0 mg, 22% yield); mp: 109.3-110.8° C.; TLC R_{f}=0.46 (EtOAc, SiO₂); IR (film, cm⁻¹) 3413, 3282, 3054, 2924, 2853, 1650, 1511, 1451, 1373, 1344, 1264, 1096; ¹H NMR (300 MHz, CDCl₃) δ (ppm) 8.23 (s, 1H), 7.38-7.28 (m, 3H), 7.20 (dd, J=8.6, 5.5 Hz, 2H), 7.09 (t, J=7.5 Hz, 1H), 6.90 (dd, J=15.1, 8.2 Hz, 2H), 6.51-6.44 (m, 1H), 5.77 (d, J=8.9 Hz, 1H), 5.37 (dt, J=8.4, 4.5 Hz, 1H), 4.23 (dd, J=8.2, 4.8 Hz, 1H), 2.29-2.19 (m, 2H), 2.07 (s, 3H), 2.06-1.95 (m, 1H), 1.72 (ddd, J=18.6, 10.8, 5.6 Hz, 1H); ¹³C NMR (75 MHz, CDCl₃) δ (ppm) 169.6 (C_{q}), 141.2 (C_{q}), 138.1 (C_{q}), 137.5 (C_{q}), 134.7 (C_{q}), 130.7 (CH), 128.0 (C_{q}), 127.8 (CH), 127.4 (CH), 126.6 (CH), 124.6 (CH), 123.1 (CH), 120.8 (CH), 111.2 (CH), 102.6 (CH), 48.2 (CH), 45.7 (CH), 31.1 (CH₂), 28.8 (CH₂), 23.8 (CH₃); HRMS (APCI) (M+Na)⁺ m/z calculated for C₂₀H₂₀N₂ONa 327.1473, found 327.1473.

### 5-(1,2,3,4-tetrahydronaphthalen-1-yl)-1H-indole [AC082]

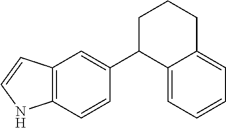 [SMILES: c1ccc2c(c1)CCCC2c1ccc2[nH]ccc2c1]

Beige solid obtained according to general procedure 1 (56.0 mg, 71% yield); mp: 117.4-119.3° C.; TLC R_{f}=0.71 (Cyclohexane/EtOAc, 7:3, SiO₂); IR (film, cm⁻¹); ¹H NMR (300 MHz, CDCl₃) δ (ppm) 8.03 (s, 1H), 7.39 (s, 1H), 7.32 (d, J=8.4 Hz, 1H), 7.16 (dd, J=11.4, 4.7 Hz, 3H), 7.07-6.91 (m, 3H), 6.51 (d, J=2.1 Hz, 1H), 4.30-4.18 (m, 1H), 2.93 (qd, J=16.4, 7.8 Hz, 2H), 2.32-2.17 (m, 1H), 2.05-1.91 (m, 2H), 1.86-1.77 (m, 1H); ¹³C NMR (75 MHz, CDCl₃) δ (ppm) 140.6 (C_{q}), 139.2 (C_{q}), 137.7 (C_{q}), 134.5 (C_{q}), 130.5 (CH), 129.0 (CH), 128.0 (C_{q}), 125.8 (CH), 125.7 (CH), 124.4 (CH), 123.4 (CH), 120.9 (CH), 110.9 (CH), 102.6 (CH), 45.9 (CH), 33.9 (CH₂), 30.1 (CH₂), 21.4 (CH₂); HRMS (APCI) (M+H)⁺ m/z calculated for C₁₈H₁₈N 248.1434, found 248.1436.

### Cis-N-(4-(isoquinolin-6-yl)-1,2,3,4-tetrahydronaphthalen-1 yl)acetamide [AC083-cis]

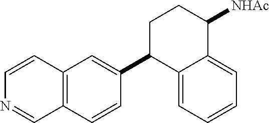

White solid obtained according to general procedure 1 (10.7 mg, 13% yield); mp: 203.3-204.5° C.; TLC R_{f}=0.53 (EtOAc, SiO₂); IR (film, cm⁻¹) 3268, 3050, 2924, 2853, 1648, 1540, 1501, 1449, 1371, 1261, 1102, 1036; ¹H NMR (300 MHz, CDCl₃) δ (ppm) 8.87 (d, J=2.8 Hz, 1H), 8.04 (t, J=8.2 Hz, 2H), 7.45 (d, J=8.7 Hz, 1H), 7.43-7.34 (m, 3H), 7.24 (d, J=7.7 Hz, 1H), 7.13 (t, J=7.4 Hz, 1H), 6.87 (d, J=7.7 Hz, 1H), 5.78 (d, J=8.6 Hz, 1H), 5.38 (dd, J=13.3, 8.1 Hz, 1H), 4.35 (t, J=6.0 Hz, 1H), 2.33-2.14 (m, 2H), 2.08 (s, 3H), 2.02 (dd, J=12.1, 9.1 Hz, 1H), 1.77 (ddd, J=12.4, 10.4, 5.1 Hz, 1H); ¹³C NMR (75 MHz, CDCl₃) δ (ppm) 169.6 (C_{q}), 150.2 (CH), 147.4 (C_{q}), 144.8 (C_{q}), 139.4 (C_{q}), 137.7 (C_{q}), 135.9 (CH), 130.8 (CH), 130.4 (CH), 129.8 (CH), 128.3 (C_{q}), 128.2 (CH), 127.7 (CH), 127.1 (2×CH), 121.4 (CH), 48.0 (CH), 45.6 (CH), 30.4 (CH₂), 28.5 (CH₂), 23.7 (CH₃); HRMS (APCI) (M+H)⁺ m/z calculated for C₂₁H₂₁N₂O 317.1648, found 317.1648.

### 6-(1,2,3,4-tetrahydronaphthalen-1-isoquinoline [AC084]

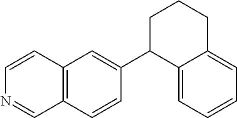 [SMILES: c1ccc2c(c1)CCCC2c1ccc2cnccc2c1]

Transparent oil obtained according to general procedure 1 (36.6 mg, 52% yield); TLC R_{f}=0.36 (Cyclohexane/EtOAc, 7:3, SiO₂); IR (film, cm⁻¹) 2958, 2924, 2853, 1734, 1595, 1495, 1467, 1264; ¹H NMR (300 MHz, CDCl₃) δ (ppm) 8.87 (d, J=3.0 Hz, 1H), 8.05 (dd, J=10.9, 8.8 Hz, 2H), 7.55-7.45 (m, 2H), 7.37 (dd, J=8.3, 4.2 Hz, 1H), 7.21-7.12 (m, 2H), 7.08-7.00 (m, 1H), 6.85 (d, J=7.6 Hz, 1H), 4.33 (t, J=6.8 Hz, 1H), 3.02-2.82 (m, 2H), 2.33-2.17 (m, 1H), 2.03-1.89 (m, 2H), 1.86-1.75 (m, 1H); ¹³C NMR (75 MHz, CDCl₃) δ (ppm) 150.0 (CH), 147.4 (C_{q}), 146.0 (C_{q}), 138.9 (C_{q}), 137.8 (C_{q}), 135.9 (CH), 131.1 (CH), 130.4 (CH), 129.5 (CH), 129.3 (CH), 128.3 (C_{q}), 127.2 (CH), 126.3 (CH), 125.9 (CH), 121.2 (CH), 45.7 (CH), 33.2 (CH₂), 29.9 (CH₂), 21.1 (CH₂); HRMS (ESI) (M+H)⁺ m/z calculated for C₁₉H₁₈N 260.1434, found 260.1432.

### 4-(naphthalen-2-yl)thiochroman [AC085]

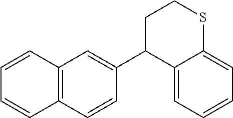 [SMILES: c1ccc2c(c1)SCCC2c1ccc2ccccc2c1]

Beige solid obtained according to general procedure 1 (134.0 mg, 49% yield); mp: 65.6-67.3° C.; TLC R_{f}=0.84 (Cyclohexane/EtOAc, 7:3, SiO₂); IR (film, cm⁻¹) 3054, 2919, 2851, 1600, 1506, 1474, 1434, 1264, 1164, 1090; ¹H NMR (300 MHz, CDCl₃) δ (ppm) 7.89-7.76 (m, 3H), 7.49 (t, J=4.7 Hz, 3H), 7.37-7.27 (m, 2H), 7.22-7.14 (m, 1H), 6.98 (q, J=7.4 Hz, 2H), 4.43 (t, J=5.1 Hz, 1H), 2.98 (dd, J=8.9, 4.2 Hz, 2H), 2.51-2.42 (m, 2H); ¹³C NMR (75 MHz, CDCl₃) δ (ppm) 142.7 (C_{q}), 135.4 (C_{q}), 133.7 (C_{q}), 133.5 (C_{q}), 132.3 (C_{q}), 131.5 (CH), 128.3 (CH), 127.9 (CH), 127.7 (CH), 127.6 (CH), 127.1 (CH), 126.8 (CH), 126.6 (CH), 126.2 (CH), 125.8 (CH), 124.2 (CH), 44.5 (CH), 30.8 (CH₂), 23.8 (CH₂).

### 6-methoxy-1,2,3,4-tetrahydro-1,2′-binaphthalene [AC086]

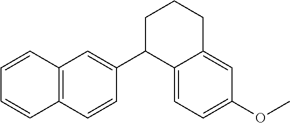 [SMILES: COc1ccc2c(c1)CCCC2c1ccc2ccccc2c1]

Beige solid obtained according to general procedure 1 (70.2 mg, 83% yield); mp: 66.6-68.3° C.; TLC R_{f}=0.80 (Cyclohexane/EtOAc, 7:3, SiO₂); IR (film, cm⁻¹) 3055, 2929, 2856, 2833, 1608, 1500, 1464, 1253, 1155, 1038; ¹H NMR (300 MHz, CDCl₃) δ (ppm) 7.92-7.81 (m, 3H), 7.63 (s, 1H), 7.52 (dd, J=6.7, 2.9 Hz, 2H), 7.35 (dd, J=8.5, 1.7 Hz, 1H), 6.88 (d, J=8.5 Hz, 1H), 6.82 (d, J=2.6 Hz, 1H), 6.71 (dd, J=8.5, 2.7 Hz, 1H), 4.31 (t, J=6.0 Hz, 1H), 3.87 (s, 3H), 3.10-2.89 (m, 2H), 2.37-2.22 (m, 1H), 2.13-1.96 (m, 2H), 1.93-1.80 (m, 1H); ¹³C NMR (75 MHz, CDCl₃) δ (ppm) 157.8 (C_{q}), 145.2 (C_{q}), 138.9 (C_{q}), 133.5 (C_{q}), 132.2 (C_{q}), 131.6 (C_{q}), 131.4 (CH), 128.0 (CH), 127.7 (2×CH), 127.3 (2×CH), 126.0 (CH), 125.4 (CH), 113.4 (CH), 112.2 (CH), 55.3 (CH), 45.3 (CH₃), 33.5 (CH₂), 30.3 (CH₂), 21.3 (CH₂); HRMS (ESI) (M+H)⁺ m/z calculated for C₂₁H₂₁O 289.1587, found 289.1588.

### N-(4-(3-iodophenyl)-1,2,3,4-tetrahydronaphthalen-1-yl)acetamide [AC087]

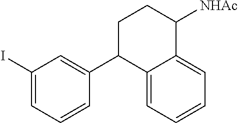 [SMILES: CC(=O)NC1CCC(c2cccc(I)c2)c2ccccc21]

Amorphous solid obtained according to general procedure 1 (47.3 mg, 47% yield, d.e=12%) mixture of diastereoisomers (obtained after trituration of the purified product, using MeOH of HPLC grade), during trituration the major dia (trans) has become a minority (and conversely for the cis dia) [cis (maj): 56%, trans (min): 44%]; TLC R_{f}=0.62 (EtOAc, SiO₂); IR (film, cm⁻¹) 3273, 3057, 2928, 2855, 1632, 1540, 1371, 1263, 1107, 1066; ¹H NMR (300 MHz, CDCl₃) δ (ppm) major dia (trans): 7.55 (d, J=6.9 Hz, 1H), 7.45 (s, 1H), 7.34 (d, J=7.5 Hz, 1H), 7.25-7.12 (m, 2H), 7.06-6.92 (m, 2H), 6.87-6.80 (m, 1H), 5.87-5.79 (m, 1H), 5.24-5.16 (m, 1H), 4.11-3.96 (m, 1H), 2.21-2.13 (m, 1H), 2.06 (s, 3H), 1.91 (dd, J=18.5, 6.5 Hz, 2H), 1.79-1.67 (m, 1H); minor dia (cis): 7.55 (d, J=6.9 Hz, 1H), 7.45 (s, 1H), 7.34 (d, J=7.5 Hz, 1H), 7.25-7.12 (m, 2H), 7.06-6.92 (m, 2H), 6.87-6.80 (m, 1H), 5.77-5.67 (m, 1H), 5.36-5.28 (m, 1H), 4.11-3.96 (m, 1H), 2.21-2.13 (m, 1H), 2.06 (s, 3H), 1.91 (dd, J=18.5, 6.5 Hz, 2H), 1.79-1.67 (m, 1H); ¹³C NMR (75 MHz, CDCl₃) δ (ppm) major dia (trans): 169.7 (C_{q}), 149.0 (C_{q}), 139.1 (C_{q}), 137.7 (CH), 137.5 (C_{q}), 135.6 (CH), 130.3 (2×CH), 129.1 (CH), 128.1 (CH), 127.7 (CH), 127.2 (CH), 94.7 (C_{q}), 47.8 (CH), 45.2 (CH), 29.6 (CH₂), 27.6 (CH₂), 23.7 (CH₃); minor dia (cis): 169.7 (C_{q}), 149.0 (C_{q}), 139.1 (C_{q}), 137.7 (CH), 137.5 (C_{q}), 135.6 (CH), 130.2 (2×CH), 129.1 (CH), 128.3 (CH), 127.9 (CH), 127.2 (CH), 94.7 (C_{q}), 48.0 (CH), 45.2 (CH), 30.5 (CH₂), 28.5 (CH₂), 23.7 (CH₃); HRMS (ESI) (M+Na)⁺ m/z calculated for C₁₈H₁₈INONa 414.0325, found 414.0328.

### N-(4-(4-bromophenyl)-1,2,3,4-tetrahydronaphthalen-1-yl)acetamide [AC088]

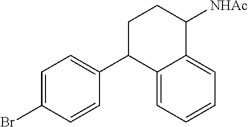 [SMILES: CC(=O)NC1CCC(c2ccc(Br)cc2)c2ccccc21]

Amorphous solid obtained according to general procedure 1 (37.6 mg, 42% yield, d.e=48%) mixture of diastereoisomers (obtained after trituration of the purified product, using HPLC grade MeOH) [cis (min): 26%, trans (maj): 74%]; TLC R_{f}=0.44 (EtOAc, SiO₂); IR (film, cm⁻¹) 3268, 3059, 2928, 2856, 1633, 1543, 1487, 1372, 1262; ¹H NMR (300 MHz, CDCl₃) δ (ppm) major dia (trans): 7.48-7.29 (m, 3H), 7.24-7.18 (m, 1H), 7.16-7.10 (m, 1H), 7.02-6.88 (m, 2H), 6.87-6.79 (m, 1H), 5.80-5.62 (m, 1H), 5.19 (dd, J=12.6, 8.1 Hz, 1H), 4.09 (dd, J=17.2, 8.5 Hz, 1H), 2.16 (dd, J=10.3, 7.6 Hz, 1H), 2.09-2.02 (s, 3H), 2.00-1.80 (m, 3H); minor dia (cis): 7.48-7.29 (m, 3H), 7.24-7.18 (m, 1H), 7.16-7.10 (m, 1H), 7.02-6.88 (m, 2H), 6.87-6.79 (m, 1H), 5.80-5.62 (m, 1H), 5.35-5.27 (m, 1H), 4.09 (dd, J=17.2, 8.5 Hz, 1H), 2.16 (dd, J=10.3, 7.6 Hz, 1H), 2.09-2.02 (s, 3H), 2.00-1.80 (m, 3H); ¹³C NMR (75 MHz, CDCl₃) δ (ppm) major dia (trans): 164.6 (C_{q}), 145.6 (C_{q}), 139.3 (C_{q}), 137.3 (C_{q}), 131.7 (2×CH), 130.6 (2×CH), 130.3 (CH), 129.1 (CH), 127.9 (CH), 127.2 (CH), 120.3 (C_{q}), 47.9 (CH), 44.9 (CH), 29.6 (CH₂), 27.5 (CH₂), 23.8 (CH₃); minor dia (cis: 164.6 (C_{q}), 145.6 (C_{q}), 139.3 (C_{q}), 137.3 (C_{q}), 131.7 (2×CH), 130.6 (2×CH), 130.3 (CH), 128.2 (CH), 127.7 (CH), 127.2 (CH), 120.3 (C_{q}), 48.0 (CH), 45.1 (CH), 30.5 (CH₂), 28.5 (CH₂), 23.8 (CH₃); HRMS (ESI) (M+Na)⁺ m/z calculated for C₁₈H₁₈BrNONa 366.0464, found 366.0478.

### N-(4-(3-vinylphenyl)-1,2,3,4-tetrahydronaphthalen-1-yl)acetamide [AC093]

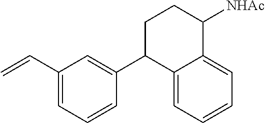 [SMILES: C=Cc1cccc(C2CCC(NC(C)=O)c3ccccc32)c1]

White solid obtained according to general procedure 1 (31.6 mg, 42% yield, d.e=52%) mixture of diastereoisomers [cis (min): 24%, trans (maj): 76%]; TLC R_{f}=0.32 (Cyclohexane/EtOAc, 1:1, SiO₂); IR (film, cm⁻¹) 3275, 3057, 2931, 2856, 1647, 1553, 1487, 1450, 1372, 1265; ¹H NMR (300 MHz, CDCl₃) δ (ppm) major dia: 7.32 (dd, J=18.0, 6.1 Hz, 2H), 7.25-7.17 (m, 2H), 7.17-7.10 (m, 2H), 6.96 (d, J=7.3 Hz, 1H), 6.92-6.86 (m, 1H), 6.68 (dt, J=17.8, 8.9 Hz, 1H), 5.93-5.82 (m, 1H), 5.71 (dd, J=17.6, 6.6 Hz, 1H), 5.23 (dd, J=11.2, 5.3 Hz, 2H), 4.08 (d, J=7.8 Hz, 1H), 2.21-2.11 (m, 1H), 2.05 (s, 3H), 2.02-1.90 (m, 3H); minor dia: 7.32 (dd, J=18.0, 6.1 Hz, 2H), 7.25-7.17 (m, 2H), 7.17-7.10 (m, 2H), 6.96 (d, J=7.3 Hz, 1H), 6.92-6.86 (m, 1H), 6.68 (dt, J=17.8, 8.9 Hz, 1H), 5.93-5.82 (m, 1H), 5.71 (dd, J=17.6, 6.6 Hz, 1H), 5.38-5.29 (m, 2H), 4.19-4.11 (m, 1H), 2.21-2.11 (m, 1H), 2.06 (s, 3H), 2.02-1.90 (m, 3H); ¹³C NMR (75 MHz, CDCl₃) δ (ppm) major dia: 169.4 (C_{q}), 146.7 (C_{q}), 139.8 (C_{q}), 137.9 (C_{q}), 137.2 (C_{q}), 137.0 (CH), 130.3 (CH), 129.0 (CH), 128.7 (CH), 128.4 (CH), 127.7 (CH), 127.0 (CH), 127.0 (CH), 124.2 (CH), 114.1 (CH₂), 48.1 (CH), 45.6 (CH), 29.5 (CH₂), 27.7 (CH₂), 23.7 (CH₃); minor dia: 169.4 (C_{q}), 146.7 (C_{q}), 139.8 (C_{q}), 137.9 (C_{q}), 137.2 (C_{q}), 137.0 (CH), 130.4 (CH), 129.0 (CH), 128.7 (CH), 128.0 (CH), 127.6 (CH), 127.0 (CH), 126.9 (CH), 124.2 (CH), 114.1 (CH₂), 47.8 (CH), 45.5 (CH), 30.6 (CH₂), 28.7 (CH₂), 23.7 (CH₃); HRMS (ESI) (M+Na)⁺ m/z calculated for C₂₀H₂₁NONa 314.1515, found 314.1518.

### N-((4-(4-(benzyloxy)-3-chlorophenyl)-1,2,3,4-tetrahydronaphthalen-1-yl) acetamide

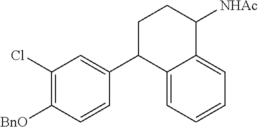 [SMILES: CC(=O)NC1CCC(c2ccc(OCc3ccccc3)c(Cl)c2)c2ccccc21]

Amorphous solid obtained according to general procedure 1 (49.1 mg, 48% yield, d.e=54%) mixture of diastereoisomers [cis (min): 23%, trans (maj): 77%]; TLC R_{f}=0.20 (Pentane/EtOAc, 1:1, SiO₂); IR (film, cm⁻¹) 2958, 2923, 2853, 1736, 1654, 1501, 1467, 1377, 1261; ¹H NMR (300 MHz, CDCl₃) δ (ppm) major dia (trans): 7.50-7.44 (m, 2H), 7.37 (ddd, J=15.2, 8.7, 1.8 Hz, 4H), 7.24-7.09 (m, 3H), 6.95-6.79 (m, 3H), 6.02-5.89 (m, 1H), 5.20 (dd, J=9.6, 4.3 Hz, 1H), 5.14 (s, 2H), 4.00 (t, J=6.3 Hz, 1H), 2.25-2.07 (m, 1H), 2.04 (s, 3H), 1.98-1.81 (m, 3H); minor dia (cis): 7.50-7.44 (m, 2H), 7.37 (ddd, J=15.2, 8.7, 1.8 Hz, 4H), 7.24-7.09 (m, 3H), 6.95-6.79 (m, 3H), 5.84-5.77 (m, 1H), 5.30 (dd, J=8.4, 5.3 Hz, 1H), 5.12 (s, 2H), 4.00 (t, J=6.3 Hz, 1H), 2.25-2.07 (m, 2H), 2.03 (s, 3H), 1.98-1.81 (m, 1H), 1.76-1.65 (m, 1H); ¹³C NMR (75 MHz, CDCl₃) δ (ppm) major dia trans): 169.5 (C_{q}), 152.8 (C_{q}), 140.2 (C_{q}), 139.5 (C_{q}), 137.2 (C_{q}), 136.7 (C_{q}), 130.4 (CH), 130.2 (CH), 129.0 (CH), 128.7 (2×CH), 128.1 (2×CH), 127.8 (CH), 127.2 (2×CH), 127.1 (CH), 123.3 (C_{q}), 114.1 (CH), 71.1 (CH₂), 47.8 (CH), 44.4 (CH), 29.6 (CH₂), 27.5 (CH₂), 23.7 (CH₃); minor dia (cis): 169.6 (C_{q}), 152.8 (C_{q}), 140.2 (C_{q}), 139.5 (C_{q}), 137.5 (C_{q}), 136.7 (C_{q}), 130.6 (CH), 130.3 (CH), 129.0 (CH), 128.7 (2×CH), 128.1 (2×CH), 127.7 (CH), 127.2 (2×CH), 127.1 (CH), 123.2 (C_{q}), 114.1 (CH), 71.1 (CH₂), 47.9 (CH), 44.5 (CH), 30.4 (CH₂), 28.4 (CH₂), 23.7 (CH₃); HRMS (ESI) (M+Na)⁺ m/z calculated for C₂₅H₂₄ClNO₂Na 428.1388, found 428.1388.

### 1-(3,4-dichlorophenyl)-6-methoxy-1,2,3,4-tetrahydronaphthalene [AC095]

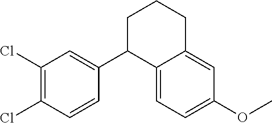 [SMILES: COc1ccc2c(c1)CCCC2c1ccc(Cl)c(Cl)c1]

Transparent oil obtained according to general procedure 1 (34.8 mg, 40% yield); TLC R_{f}=0.76 (Cyclohexane/EtOAc, 7:3, SiO₂); IR (film, cm⁻¹) 2932, 2859, 2834, 1609, 1576, 1501, 1466, 1255, 1124, 1041; ¹H NMR (300 MHz, CDCl₃) δ (ppm) 7.33 (d, J=8.3 Hz, 1H), 7.18 (d, J=2.0 Hz, 1H), 6.92 (dd, J=8.2, 2.0 Hz, 1H), 6.76-6.61 (m, 3H), 4.03 (t, J=6.3 Hz, 1H), 3.79 (s, 3H), 2.92-2.75 (m, 2H), 2.22-2.08 (m, 1H), 1.88-1.65 (m, 3H); ¹³C NMR (75 MHz, CDCl₃) δ (ppm) 158.1 (C_{q}), 148.3 (C_{q}), 138.9 (C_{q}), 132.3 (C_{q}), 131.1 (CH), 130.7 (CH), 130.3 (C_{q}), 130.3 (CH), 129.9 (C_{q}), 128.3 (CH), 113.6 (CH), 112.5 (CH), 55.3 (CH), 44.3 (CH₃), 33.4 (CH₂), 30.1 (CH₂), 20.8 (CH₂); HRMS (ESI) (M+H)⁺ m/z calculated for C₁₇H₁₇C₁₂O 307.0651, found 307.0508.

### 1-(3,4-dichlorophenyl)-1,2,3,4-tetrahydronaphthalene [AC096]

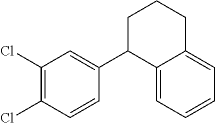 [SMILES: Clc1ccc(C2CCCc3ccccc32)cc1Cl]

Transparent oil obtained according to general procedure 1 (61.5 mg, 70% yield); TLC R_{f}=0.89 (Cyclohexane/EtOAc, 7:3, SiO₂); IR (film, cm⁻¹) 2932, 2858, 1588, 1560, 1491, 1468, 1448, 1395, 1130, 1030; ¹H NMR (300 MHz, CDCl₃) δ (ppm) 7.35 (d, J=8.2 Hz, 1H), 7.20 (d, J=1.9 Hz, 1H), 7.16 (d, J=4.2 Hz, 2H), 7.06 (dt, J=8.4, 4.3 Hz, 1H), 6.93 (dd, J=8.2, 2.0 Hz, 1H), 6.81 (d, J=7.6 Hz, 1H), 4.10 (t, J=6.4 Hz, 1H), 2.97-2.77 (m, 2H), 2.24-2.06 (m, 1H), 1.94-1.71 (m, 3H); ¹³C NMR (75 MHz, CDCl₃) δ (ppm) 148.0 (C_{q}), 138.1 (C_{q}), 137.7 (C_{q}), 132.3 (C_{q}), 130.8 (CH), 130.3 (CH), 130.1 (CH), 130.0 (C_{q}), 129.4 (CH), 128.4 (CH), 126.5 (CH), 126.0 (CH), 45.0 (CH), 33.2 (CH₂), 29.7 (CH₂), 20.9 (CH₂).

### 1-(3-chlorophenyl)-6-methoxy-1,2,3,4-tetrahydronaphthalene [AC101]

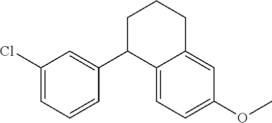 [SMILES: COc1ccc2c(c1)CCCC2c1cccc(Cl)c1]

Transparent oil obtained according to general procedure 1 (47.9 mg, 60% yield); TLC R_{f}=0.80 (Cyclohexane/EtOAc, 7:3, SiO₂); IR (film, cm⁻¹) 2932, 2857, 2834, 1610, 1593, 1573, 1501, 1466, 1428, 1256, 1039; ¹H NMR (300 MHz, CDCl₃) δ (ppm) 7.24-7.14 (m, 2H), 7.09 (s, 1H), 6.98 (d, J=6.7 Hz, 1H), 6.74 (d, J=8.4 Hz, 1H), 6.69-6.58 (m, 2H), 4.05 (t, J=6.2 Hz, 1H), 3.79 (s, 3H), 2.94-2.76 (m, 2H), 2.20-2.06 (m, 1H), 1.90-1.71 (m, 3H); ¹³C NMR (75 MHz, CDCl₃) δ (ppm) 157.9 (C_{q}), 150.0 (C_{q}), 138.9 (C_{q}), 134.7 (C_{q}), 131.2 (CH), 130.8 (C_{q}), 129.6 (CH), 128.9 (CH), 127.1 (CH), 126.2 (CH), 113.5 (CH), 112.4 (CH), 55.3 (CH₃), 44.8 (CH₃), 33.5 (CH₂), 30.2 (CH₂), 20.9 (CH₂); HRMS (ESI) (M+H)⁺ m/z calculated for C₁₇H₁₈ClO 273.1041, found 273.1046.

### (2R,3R,4S,5R,6S)-2-(acetoxymethyl)-6-((3-(1,2,3,4-tetrahydronaphthalen-1yl)phenyl)thio)tetrahydro-2H-pyran-3,4,5-triyl triacetate [AC116]

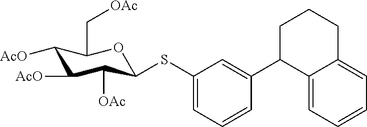

White solid obtained according to general procedure 2 (169.9 mg, 90% yield, d.e=0%) mixture of diastereoisomers [dia 1: 50%, dia 2: 50%]; TLC R_{f}=0.53 (Cyclohexane/EtOAc, 6:4, SiO₂); IR (film, cm⁻¹) 2929, 2854, 1756, 1590, 1366, 1248, 1213, 1091, 1036; ¹H NMR (300 MHz, CDCl₃) δ (ppm) dia 1: 7.30 (d, J=7.5 Hz, 1H), 7.20 (dd, J=15.1, 4.4 Hz, 2H), 7.13 (d, J=4.0 Hz, 2H), 7.09-7.01 (m, 2H), 6.83 (dd, J=7.2, 2.7 Hz, 1H), 5.20 (td, J=9.3, 2.7 Hz, 1H), 5.11-4.89 (m, 2H), 4.67 (dd, J=13.7, 10.1 Hz, 1H), 4.22-4.06 (m, 2H), 4.06-3.93 (m, 1H), 3.70-3.64 (m, 1H), 2.86 (tt, J=16.8, 8.3 Hz, 2H), 2.21-2.10 (m, 1H), 2.08-1.96 (m, 12H), 1.92-1.81 (m, 2H), 1.81-1.71 (m, 1H); dia 2: 7.30 (d, J=7.5 Hz, 1H), 7.20 (dd, J=15.1, 4.4 Hz, 2H), 7.13 (d, J=4.0 Hz, 2H), 7.09-7.01 (m, 2H), 6.83 (dd, J=7.2, 2.7 Hz, 1H), 5.20 (td, J=9.3, 2.7 Hz, 1H), 5.11-4.89 (m, 2H), 4.67 (dd, J=13.7, 10.1 Hz, 1H), 4.22-4.06 (m, 2H), 4.06-3.93 (m, 1H), 3.56 (dd, J=9.8, 2.4 Hz, 1H), 2.86 (tt, J=16.8, 8.3 Hz, 2H), 2.21-2.10 (m, 1H), 2.08-1.96 (m, 12H), 1.92-1.81 (m, 2H), 1.81-1.71 (m, 1H); ¹³C NMR (75 MHz, CDCl₃) δ (ppm) dia 1: 170.19 (2×C_{q}), 169.37 (2×C_{q}), 148.76 (C_{q}), 138.71 (C_{q}), 137.73 (C_{q}), 132.42 (CH), 131.54 (C_{q}), 130.17 (2×CH), 129.11 (CH), 128.76 (2×CH), 126.16 (CH), 125.76 (CH), 85.65 (CH), 75.72 (CH), 74.04 (CH), 69.83 (CH), 68.08 (CH), 62.05 (CH₂), 45.38 (CH), 33.21 (CH₂), 29.73 (CH₂), 20.87 (CH₂), 20.63 (4×CH₃); dia 2: 170.58 (2×C_{q}), 169.23 (C_{q}), 169.14 (C_{q}), 148.63 (C_{q}), 138.71 (C_{q}), 137.59 (C_{q}), 132.95 (CH), 132.29 (C_{q}), 130.24 (CH), 129.75 (CH), 129.16 (CH), 128.90 (CH), 128.84 (CH), 126.23 (CH), 125.87 (CH), 86.25 (CH), 75.90 (CH), 74.07 (CH), 69.90 (CH), 68.24 (CH), 62.26 (CH₂), 45.44 (CH), 33.21 (CH₂), 29.73 (CH₂), 20.87 (CH₂), 20.76 (4×CH₃); HRMS (ESI) (M+Na)⁺ m/z calculated for C₃₀H₃₄O₉SNa 593.1826, found 593.1829.

### (2R,3S,4S,5R,6S)-2-(hydroxymethyl)-6-((3-(1,2,3,4-tetrahydronaphthalen-1-yl)phenyl)thio) tetrahydro-2H-pyran-3,4,5-triol [AC121]

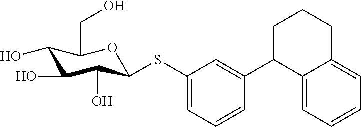

Compound prepared according to general procedure 2, followed by a deprotection reaction using sodium methanolate [sodium (0.26 mmol, 1.5 eq) dissolved in methanol (0.6 mL)]. Then, the reaction medium is stirred at room temperature for 30 minutes before being acidified with DOWEX® 50WX8-200 for 20 minutes. The crude reaction mixture is then filtered and concentrated under vacuum to give a light brown solid (68.6 mg, 98% yield, d.e=24%) mixture of diastereoisomers [dia 1: 60%, dia 2: 38%]; TLC R_{f}=0.17 (EtOAc, SiO₂); IR (film, cm⁻¹) 3357, 2926, 2854, 1589, 1451, 1418, 1275, 1022; ¹H NMR (300 MHz, MeOD) δ (ppm) dia 1: 7.39 (t, J=7.1 Hz, 1H), 7.32-7.20 (m, 2H), 7.15-7.07 (m, 2H), 7.06-6.98 (m, 2H), 6.80 (s, 1H), 4.86 (s, 4H), 4.56 (dd, J=12.1, 9.8 Hz, 1H), 4.13 (t, J=6.4 Hz, 1H), 3.82-3.77 (m, 1H), 3.69-3.56 (m, 1H), 3.44-3.31 (m, 2H), 3.30-3.16 (m, 2H), 2.87 (qd, J=16.7, 7.8 Hz, 2H), 2.20-2.09 (m, 1H), 1.96-1.81 (m, 2H), 1.81-1.68 (m, 1H); dia 2: 7.39 (t, J=7.1 Hz, 1H), 7.32-7.20 (m, 2H), 7.15-7.07 (m, 2H), 7.06-6.98 (m, 2H), 6.78 (s, 1H), 4.86 (s, 4H), 4.56 (dd, J=12.1, 9.8 Hz, 1H), 4.13 (t, J=6.4 Hz, 1H), 3.77-3.72 (m, 1H), 3.69-3.56 (m, 1H), 3.44-3.31 (m, 2H), 3.30-3.16 (m, 2H), 2.87 (qd, J=16.7, 7.8 Hz, 2H), 2.20-2.09 (m, 1H), 1.96-1.81 (m, 2H), 1.81-1.68 (m, 1H); ¹³C NMR (75 MHz, CDCl₃) δ (ppm) dia 1: 148.6 (C_{q}), 138.8 (C_{q}), 137.6 (C_{q}), 132.9 (C_{q}), 131.9 (CH), 130.2 (CH), 129.1 (2×CH), 128.3 (CH), 126.2 (2×CH), 125.8 (CH), 88.1 (CH), 79.4 (CH), 77.9 (CH), 72.4 (CH), 69.3 (CH), 61.7 (CH₂), 45.4 (CH), 33.3 (CH₂), 29.8 (CH₂), 20.9 (CH₂); dia 2: 148.7 (C_{q}), 138.8 (C_{q}), 137.7 (C_{q}), 133.3 (C_{q}), 131.9 (CH), 130.3 (CH), 129.1 (2×CH), 128.3 (CH), 126.2 (2×CH), 125.9 (CH), 88.6 (CH), 79.5 (CH), 77.9 (CH), 72.4 (CH), 69.2 (CH), 61.7 (CH₂), 45.4 (CH), 33.3 (CH₂), 29.8 (CH₂), 20.9 (CH₂); HRMS (ESI) (M+Na)⁺ m/z calculated for C₂₂H₂₆05SNa 425.1393, found 425.1399.

### 4-(4-methoxyphenyl)chroman[AC147]

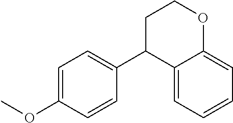 [SMILES: COc1ccc(C2CCOc3ccccc32)cc1]

White solid obtained according to general procedure 1 (229.8 mg, 95% yield); mp: 90.3-90.7° C.; TLC R_{f}=0.70 (Cyclohexane/EtOAc, 7:3, SiO₂); IR (film, cm⁻¹) 2952, 2877, 2834, 1610, 1581, 1511, 1487, 1452, 1304, 1269, 1249; ¹H NMR (300 MHz, CDCl₃) δ (ppm) 7.19-7.12 (m, 1H), 7.09 (d, J=8.6 Hz, 2H), 6.92-6.79 (m, 5H), 4.19 (dt, J=12.7, 5.8 Hz, 3H), 3.82 (s, 3H), 2.31 (ddd, J=13.4, 10.6, 5.6 Hz, 1H), 2.09 (ddd, J=10.9, 9.4, 4.9 Hz, 1H); ¹³C NMR (75 MHz, CDCl₃) δ (ppm) 158.3 (C_{q}), 155.2 (C_{q}), 137.8 (C_{q}), 130.7 (CH), 129.7 (2×CH), 127.9 (CH), 125.0 (C_{q}), 120.4 (CH), 116.8 (CH), 114.0 (2×CH), 64.0 (CH₂), 55.4 (CH), 40.3 (CH₃), 31.9 (CH₂); HRMS (ESI) (M+H)⁺ m/z calculated for C₁₆H₁₇O₂ 241.1223, found 241.1229.

### Cis-(2S,3R,4S,5R,6R)-2-((5-((4S)-4-acetamido-1,2,3,4-tetrahydronaphthalen-1-yl)-2-(quinolin-8-ylcarbamoyl)-3-(((2R,3S,4R,5S,6S)-3,4,5-triacetoxy-6-(acetoxymethyl)tetrahydro-2H-pyran-2-yl)thio) phenyl)thio)-6-(acetoxymethyl)tetrahydro-2H-pyran-3,4,5-triyl triacetate [AC249-cis/AC339-HPLC2]

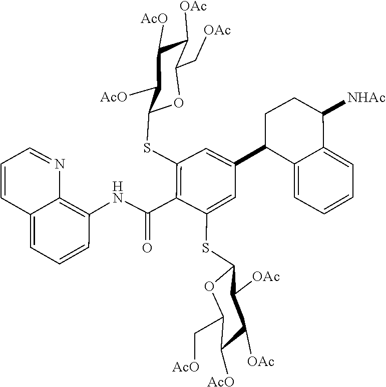

White solid obtained according to general procedure 4 (113.8 mg, 61% yield); mp: 153.8-155.4° C.; TLC R_{f}=0.40 (EtOAc, SiO₂); IR (film, cm⁻¹) 3334, 1755, 1649, 1524, 1367, 1212, 1036; ¹H NMR (300 MHz, CDCl₃) δ (ppm) 9.82 (d, J=5.5 Hz, 1H), 8.94-8.85 (m, 1H), 8.79 (t, J=3.5 Hz, 1H), 8.18 (d, J=8.2 Hz, 1H), 7.55 (d, J=4.7 Hz, 2H), 7.52-7.47 (m, 1H), 7.45 (d, J=6.4 Hz, 2H), 7.39 (d, J=7.4 Hz, 1H), 7.33-7.26 (m, 1H), 7.21 (dd, J=14.4, 7.3 Hz, 1H), 6.92 (dd, J=14.4, 7.4 Hz, 1H), 5.74 (d, J=8.5 Hz, 1H), 5.38-5.27 (m, 1H), 5.13-4.99 (m, 3H), 4.95 (d, J=10.0 Hz, 1H), 4.89 (dd, J=6.8, 2.4 Hz, 1H), 4.87-4.80 (m, 2H), 4.75 (t, J=8.1 Hz, 1H), 4.26-4.18 (m, 1H), 4.16-4.04 (m, 2H), 3.99 (dd, J=12.2, 1.7 Hz, 1H), 3.83 (dd, J=12.0, 0.9 Hz, 1H), 3.67-3.57 (m, 1H), 3.50 (d, J=9.4 Hz, 1H), 2.33-2.17 (m, 2H), 2.05 (s, 3H), 2.02 (s, 3H), 2.01 (s, 3H), 2.00 (s, 3H), 1.99 (s, 3H), 1.93 (s, 3H), 1.91 (s, 3H), 1.85-1.70 (m, 2H), 1.57 (s, 3H), 1.47 (s, 3H); ¹³C NMR (75 MHz, CDCl₃) δ (ppm) 170.6 (2×C_{q}), 170.1 (3×C_{q}), 169.5 (4×C_{q}), 165.0 (C_{q}, split 164.9), 149.1 (C_{q}, split 148.9), 148.7 (CH), 143.8 (C_{q}, split 143.6), 138.7 (C_{q}), 138.6 (C_{q}, split 138.5), 138.0 (C_{q}, split 137.8), 136.49 (CH, split 136.38), 136.2 (CH), 134.4 (C_{q}, split 134.3), 130.4 (C_{q}), 130.3 (CH, split 130.2), 130.0 (C_{q}), 128.59 (CH, split 128.4), 128.2 (C_{q}), 127.8 (CH), 127.5 (CH), 127.3 (CH), 122.4 (CH), 122.2 (CH, split 122.1), 117.1 (CH), 86.9 (CH), 86.3 (CH), 76.0 (CH), 75.7 (CH), 74.1 (CH), 73.9 (CH), 69.8 (CH), 69.6 (CH), 68.3 (CH), 68.0 (CH), 61.9 (CH₂), 61.54 (CH₂), 47.8 (CH), 45.2 (CH, split 45.1), 28.4 (CH₂), 28.1 (CH₂), 23.7 (CH₃), 20.8 (3×CH₃), 20.7 (3×CH₃), 20.3 (CH₃), 20.1 (CH₃); HRMS (APCI) (M+H)⁺ m/z calculated for C₅₆H₆₂N₃O₂₀S₂ 1160.3363, found 1160.3368.

### Cis-(2S,3R,4S,5R,6R)-2-((3-((4S)-4-acetamido-1,2,3,4-tetrahydronaphthalen-1-yl)phenyl)thio)-6-(acetoxymethyl)tetrahydro-2H-pyran-3,4,5-triyl triacetate [AC268-cis/AC268-HPLC3]

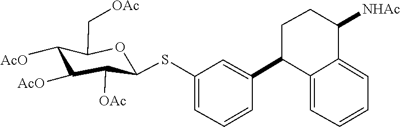

White solid obtained according to general procedure 2 (18.0 mg, 23% yield); Purified by HPLC using an Xbridge C18 type column (4.6×150 mm, 5 μm) and a H₂O/MeOH mixture (40:60) as solvent; mp: 232.9-234.3° C.; TLC R_{f}=0.46 (EtOAc, SiO₂); IR (film, cm⁻¹) 3371, 2927, 1756, 1650, 1540, 1368, 1227, 1036; ¹H NMR (300 MHz, CDCl₃) δ (ppm) 7.38-7.27 (m, 3H), 7.24-7.14 (m, 5H), 6.85-6.77 (m, 1H), 6.61 (d, J=8.1 Hz, 1H), 5.27-5.20 (m, 1H), 5.15 (t, J=9.3 Hz, 1H), 5.00 (t, J=9.7 Hz, 1H), 4.85 (t, J=9.6 Hz, 1H), 4.57 (d, J=10.1 Hz, 1H), 4.05 (t, J=5.9 Hz, 1H), 3.96 (dd, J=12.3, 2.3 Hz, 1H), 3.79 (dd, J=12.4, 3.6 Hz, 1H), 3.41 (dt, J=10.1, 3.0 Hz, 1H), 2.24-2.09 (m, 3H), 2.05 (s, 9H), 2.02 (s, 3H), 1.98 (s, 3H); ¹³C NMR (75 MHz, CDCl₃) δ (ppm) 170.9 (C_{q}), 170.3 (C_{q}), 169.7 (C_{q}), 169.6 (C_{q}), 169.4 (C_{q}), 148.0 (C_{q}), 139.9 (C_{q}), 137.6 (C_{q}), 132.7 (CH), 132.3 (C_{q}), 131.0 (CH), 130.1 (CH), 129.5 (CH), 129.3 (CH), 128.8 (CH), 127.5 (CH), 126.9 (CH), 85.9 (CH), 75.6 (CH), 74.0 (CH), 70.1 (CH), 68.0 (CH), 61.2 (CH₂), 47.7 (CH), 45.5 (CH), 29.5 (CH₂), 27.7 (CH₂), 23.4 (CH₃), 20.9 (2×CH₃), 20.7 (2×CH₃); HRMS (ESI) (M+Na)⁺ m/z calculated for C₃₂H₃₇NO₁₀SNa 650.2036, found 650.2042.

### (2R)-2-acetamido-3-((3-((1R)-4-acetamido-1,2,3,4-tetrahydronaphthalen-1-yl) phenyl)thio) propanoic acid [AC300]

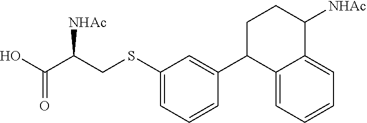 [SMILES: CC(=O)NC1CCC(c2cccc(SC[C@H](NC(C)=O)C(=O)O)c2)c2ccccc21]

White solid obtained according to general procedure 2 (62.2 mg, 95% yield), mixed diastereoisomers, but the d.e could not be determined on the ¹H proton NMR spectrum; TLC R_{f}=0.03 (DCM/MeOH, 9:1, SiO₂); IR (film, cm⁻¹) 3376, 2932, 2278, 1630, 1545, 1404; ¹H NMR (300 MHz, MeOD) δ (ppm) 7.29-6.93 (m, 8H), 6.77 (d, J=7.5 Hz, 1H), 5.14-5.01 (m, 1H), 4.44-4.32 (m, 1H), 4.02 (t, J=6.3 Hz, 1H), 3.52-3.37 (m, 1H), 3.29-3.25 (m, 1H), 3.18-3.08 (m, 1H), 2.11-2.00 (m, 1H), 2.00 (s, 3H), 1.99-1.93 (m, 1H), 1.88 (s, 3H); ¹³C NMR (75 MHz, MeOD) δ (ppm) 177.1 (C_{q}), 172.7 (C_{q}), 172.6 (C_{q}), 149.0 (C_{q}), 140.9 (C_{q}), 140.8 (C_{q}), 138.3 (C_{q}), 138.1 (C_{q}), 137.9 (C_{q}), 131.6 (CH), 131.1 (CH), 130.8 (CH), 129.9 (CH), 129.8 (CH), 129.1 (CH), 128.4 (CH), 127.9 (CH), 127.6 (CH), 56.0 (CH), 55.9 (CH), 48.9 (CH), 48.8 (CH), 46.6 (CH), 38.2 (CH₂), 37.4 (CH₂), 30.5 (CH₂), 28.5 (CH₂), 28.3 (CH₂), 22.7 (2×CH₃); HRMS (ESI) (M+Na)⁺ m/z calculated for C₂₃H₂₆N₂O₄SNa 449.1511, found 449.1511.

### Cis-(2S,3R,4S,5R,6R)-2-((4-((4S)-4-acetamido-1,2,3,4-tetrahydronaphthalen-1-yl)benzoyl)oxy)-6-(acetoxymethyl)tetrahydro-2H-pyran-3,4,5-triyl triacetate [AC435]

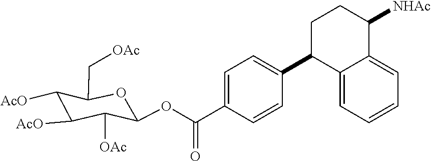 [SMILES: CC(=O)N[C@@H]1CC[C@H](c2ccc(C(=O)OC3OC(COC(C)=O)C(OC(C)=O)C(OC(C)=O)C3OC(C)=O)cc2)c2ccccc21]

Using the compound cis-4-(4-acetamido-1,2,3,4-tetrahydronaphthalen-1-yl)benzoic acid [VM055-cis] as starting material, the final compound is prepared according to general procedure 5 to obtain a white solid 68.5 mg, 55% yield); mp: 120.1-122.3° C.; TLC R_{f}=0.45 (EtOAC, SiO₂); IR (film, cm⁻¹) 3280, 2934, 1759, 1652, 1367, 1272, 1242, 1067, 1034; ¹H NMR (300 MHz, CDCl₃) δ (ppm) 7.95 (d, J=8.1 Hz, 2H), 7.35 (d, J=7.7 Hz, 1H), 7.28-7.19 (m, 1H), 7.13 (d, J=8.1 Hz, 3H), 6.77 (d, J=9.5 Hz, 1H), 5.93-5.87 (m, 1H), 5.75 (d, J=8.1 Hz, 1H), 5.37-5.27 (m, 2H), 5.22-5.13 (m, 1H), 4.30 (dd, J=12.7, 4.6 Hz, 1H), 4.21 (t, J=6.7 Hz, 1H), 4.12 (dd, J=8.8, 4.9 Hz, 1H), 3.96-3.87 (m, 1H), 2.30-2.10 (m, 2H), 2.06 (s, 6H), 2.04 (s, 3H), 2.03 (s, 3H), 1.99 (d, J=2.9 Hz, 3H), 1.97-1.83 (m, 2H), 1.79-1.63 (m, 1H); ¹³C NMR (75 MHz, CDCl₃) δ (ppm) 170.73 (C_{q}), 170.20 (C_{q}), 169.55 (2×C_{q}), 169.45 (C_{q}), 164.48 (C_{q}), 153.23 (C_{q}), 138.90 (C_{q}), 137.63 (C_{q}), 130.54 (2×CH), 130.25 (CH), 129.13 (2×CH), 128.24 (CH), 127.74 (CH), 127.27 (CH), 126.70 (C_{q}), 92.36 (CH), 72.85 (2×CH), 70.30 (CH), 68.08 (CH), 61.64 (CH₂), 47.88 (CH), 45.67 (CH), 30.28 (CH₂), 28.31 (CH₂), 23.69 (CH₃), 20.81 (CH₃), 20.71 (3×CH₃); HRMS (ESI) (M+Na)⁺ m/z calculated for C₃₃H₃₇NO₁₂Na 662.2238, found 662.2212.

### Cis-Ethyl 4-((4S)-4-acetamido-1,2,3,4-tetrahydronaphthlen-1-yl)benzoate [VM039-cis]

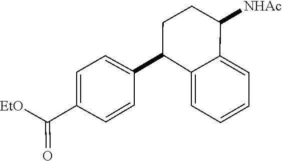 [SMILES: CCOC(=O)c1ccc([C@H]2CC[C@@H](NC(C)=O)c3ccccc32)cc1]

The compound is prepared according to general procedure 3, followed by a hydrogenation reaction using 10% by weight of palladium on charcoal (0.58 mmol, 0.15 eq) dissolved in methanol (40.0 mL). The medium is hydrogenated for 19 hours under atmospheric hydrogen pressure (hydrogenation apparatus made to specifications in the laboratory). When the reaction is complete, the reaction crude is filtered on a celite block and the solvent is evaporated under reduced pressure in the rotary evaporator. Purification by HPLC using an Xbridge C18 column (4.6×150 mm, 5 μm) and a mixture of H₂O+0.1% AF/ACN (gradient from 55% to 45% ACN in 15 minutes) as solvent, resulted in a white solid (329.0 mg, 25% yield); mp: 175.0° C.; TLC R_{f}=0.57 (Toluene/Acetone, 7:3, SiO₂); IR (film, cm⁻¹) 3280, 3060, 2935, 2859, 1716, 1640, 1539, 1369, 1275, 1104; ¹H NMR (400 MHz, CDCl₃) δ (ppm) 7.97 (d, J=10.8 Hz, 2H), 7.37 (d, J=10.8 Hz, 1H), 7.24 (t, J=10.0 Hz, 1H), 7.13 (d, J=11.2 Hz, 3H), 6.82 (d, J=10.0 Hz, 1H), 5.78 (d, J=11.6 Hz, 1H), 5.39-5.31 (m, 1H), 4.37 (q, J=9.6 Hz, 2H), 4.24-4.20 (m, 1H), 2.30-2.21 (m, 2H), 2.08 (s, 3H), 2.01-1.87 (m, 1H), 1.85-1.70 (m, 1H), 1.39 (t, J=9.6 Hz, 3H); ¹³C NMR (100 MHz, CDCl₃) δ (ppm) 169.5 (C_{q}), 166.5 (C_{q}), 151.7 (C_{q}), 139.1 (C_{q}), 137.5 (C_{q}), 130.2 (C_{q}), 129.7 (2×CH), 128.7 (2×CH), 128.0 (CH), 127.5 (CH), 127.0 (CH), 127.0 (CH), 60.9 (CH₂), 47.8 (CH), 45.5 (CH), 30.3 (CH₂), 28.3 (CH₂), 23.5 (CH₃), 14.3 (CH₃); HRMS (ESI) (M+H)⁺ m/z calculated for C₂₁H₂₄NO₃ 338.1751, found 338.1756.

### Cis-1,2,3,4-tetrahydro-[1,2′-binaphthalen]-4-amine hydrochloride [VM045-cis]

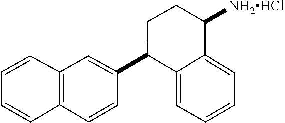

Prepared according to general procedure 1, the compound (0.634 mmol, 1.0 eq) is then dissolved in anhydrous tetrahydrofuran (THF) (2 mL, 0.317 M). The reaction medium is degassed with argon and then cooled to 0° C. In a second step, pyridine (0.757 mmol, 1.2 eq) and oxalyl chloride (0.694 mmol, 1.1 eq) are added dropwise to the reaction medium (appearance of a yellow suspension). The mixture is stirred at 0° C. for 15 minutes, then propylene glycol (92 μL, 1.26 mmol, 2 eq) is added. Propan-1-ol (5 320 mmol, 8.0 eq) and HCl (4 M in dioxane) (2 400 mmol, 3.8 eq) are added in turn. Finally, the reaction medium is stirred at room temperature overnight. Once the reaction is complete, the solvents are removed under reduced pressure and the crude is then triturated with cold methyl tert-butyl ether (MTBE). The resulting solid is then filtered and rinsed with cold MTBE. Thus, the white solid obtained corresponds to the expected final product (145.0 mg, 74% yield); mp: 276° C. (decomposition); TLC R_{f}=0.05 (DCM/MeOH, 95:5, SiO₂); IR (film, cm⁻¹) 2900, 2613, 1610, 1574, 1508, 1457, 1127; ¹H NMR (400 MHz, MeOD) δ (ppm) 8.65 (bs, ω_{1/2}=31 Hz, 2H), 7.94-7.77 (m, 2H), 7.73 (d, J=10.4 Hz, 1H), 7.56 (s, 1H), 7.54-7.42 (m, 2H), 7.33 (t, J=9.6 Hz, 1H), 7.28-7.18 (m, 2H), 6.87 (d, J=10.4 Hz, 1H), 4.64 (bs, ω_{1/2}=15 Hz, 1H), 4.37 (bs, ω_{1/2}=15 Hz, 1H), 2.42-2.14 (m, 2H), 2.04-1.81 (m, 2H), pas de protons du NH₂ observés; ¹³C NMR (15 MHz, MeOD) δ (ppm) 146.3 (C_{q}), 142.5 (C_{q}), 136.2 (C_{q}), 135.6 (C_{q}), 134.4 (C_{q}), 133.0 (CH), 131.0 (CH), 130.7 (CH), 130.6 (CH), 130.2 (2×CH), 129.6 (2×CH), 129.3 (CH), 128.9 (CH), 128.3 (CH), 50.9 (CH), 47.0 (CH), 30.8 (CH₂), 28.3 (CH₂); HRMS (ESI) (M+H)⁺ m/z calculated for C₂₀H₂₁ClN 274.1590, found 274.1601.

### Cis-4-(4-acetamido-1,2,3,4-tetrahydronaphthalen-1-yl)benzoic acid [VM055-cis]

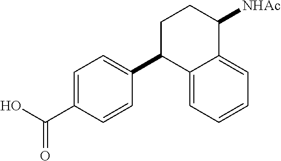 [SMILES: CC(=O)N[C@@H]1CC[C@H](c2ccc(C(=O)O)cc2)c2ccccc21]

Prepared according to general procedure 1, the compound then undergoes a hydrolysis reaction using sodium hydroxide (0.59 mmol, 2.0 eq) dissolved in ethanol (1 mL). The reaction medium is then stirred at 50° C. for 15 hours, diluted with water (20 mL) and acidified to pH=1 with HCl (37% concentration). The expected product precipitates during the HCl addition phase. It is then extracted twice with ethyl acetate (EtOAc). The combined organic phases are washed with water, followed by a saturated aqueous solution of NaCl, then dried over MgSO₄, filtered and evaporated, using a rotary evaporator, under reduced pressure. The expected product is recovered as a beige solid (80.2 mg, 87% yield); mp: 282° C. (decomposition); TLC R_{f}=0.01 (DCM/MeOH, 95:5, SiO₂); IR (film, cm⁻¹) 3324, 2926, 1698, 1610, 1542, 1451, 1376, 1241, 1179; ¹H NMR (400 MHz, MeOD) δ (ppm) 7.96 (d, J=10.8 Hz, 2H), 7.31 (d, J=10.4 Hz, 1H), 7.25-7.18 (m, 3H), 7.12 (t, J=9.6 Hz, 1H), 6.80 (d, J=10.4 Hz, 1H), 5.24 (t, J=8.4 Hz, 1H), 4.29 (t, J=8.8 Hz, 1H), 2.33-2.27 (m, 1H), 2.19-2.08 (m, 1H), 2.05 (s, 3H), 1.98-1.88 (m, 1H), 1.82-1.76 (m, 1H), pas de protons de CO₂H et de NH observés sur le spectre NMR ¹H; ¹³C NMR (100 MHz, MeOD) δ (ppm) 172.7 (C_{q}), 169.8 (C_{q}), 153.7 (C_{q}), 140.4 (C_{q}), 138.8 (C_{q}), 131.1 (C_{q}), 130.9 (2×CH), 129.9 (2×CH), 129.1 (CH), 128.4 (CH), 127.8 (CH), 128.9 (C_{q}), 46.7 (2×CH), 31.5 (CH₂), 29.2 (CH₂), 22.7 (CH₃); HRMS (ESI) (M+Na)⁺ m/z calculated for C₁₉H₁₉NO₃Na 332.1257, found 332.1256.

### Cis-(2R,3R,4S,5R,6R)-2-(4-acetamido-1,2,3,4-tetrahydronaphthalen-1-yl)benzamido)-6-(acetoxy-methyl)tetrahydro-2H-pyran-3,4,5-triyl triacetate [VM060-cis]

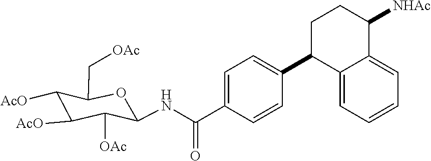 [SMILES: CC(=O)N[C@@H]1CC[C@H](c2ccc(C(=O)NC3OC(COC(C)=O)C(OC(C)=O)C(OC(C)=O)C3OC(C)=O)cc2)c2ccccc21]

A solution of the corresponding cis-benzoic acid (009 mmol, 1.0 eq) (obtained by general procedure 1, followed by a hydrolysis reaction) is prepared in anhydrous N,N-dimethylformamide (DMF) (1 mL). HOBt (0.118 mmol, 1.3 eq) and EDC.HCl (0.120 mmol, 1.3 eq) are then added to this solution. The whole is stirred for 30 minutes at room temperature. In a second step, the compound (2R,3R,4S,5R,6R)-2-(acetoxymethyl)-6-aminotetrahydro-2H-pyran-3,4,5-triyl triacetate (0.118 mmol, 1.3 eq) is added in a single step to the reaction medium and the whole is stirred at room temperature for 15 hours. When the reaction is complete, the reaction is diluted with a saturated aqueous solution of NH₄Cl and extracted three times with ethyl acetate (EtOAc). The combined organic phases are washed with water, followed by a saturated aqueous solution of NaCl, then dried over MgSO₄, filtered and evaporated, using a rotary evaporator, under reduced pressure. The expected product is recovered as a white solid (36 mg, 62% yield); mp: 209° C.; TLC R_{f}=0.45 (DCM/MeOH, 94:6, SiO₂); IR (film, cm⁻¹) 3304, 2929, 1756, 1650, 1536, 1366, 1210, 1033; ¹H NMR (400 MHz, MeOD) δ (ppm) 7.77 (d, J=10.4 Hz, 2H), 7.67 (d, J=10.8 Hz, 2H), 7.35 (d, J=10.0 Hz, 1H), 7.22 (t, J=10.0 Hz, 1H), 7.11 (d, J=10.4 Hz, 3H), 6.78 (d, J=10.0 Hz, 1H), 6.08 (t, J=8.0 Hz, 1H), 5.99-5.88 (m, 1H), 5.56-5.23 (m, 3H), 5.16-4.99 (m, 1H), 4.35-4.25 (m, 1H), 4.25-4.14 (m, 1H), 4.10 (d, J=16.0 Hz, 1H), 3.89 (d, J=13.6 Hz, 1H), 2.30-2.10 (m, 2H), 2.16 (s, 3H), 2.06 (s, 6H), 2.04 (s, 6H), 1.98-1.80 (m, 2H), les proton de NHAc et de NHCO ne sont pas observés sur le spectre NMR ¹H; ¹³C NMR (100 MHz, MeOD) δ (ppm) 171.4 (C_{q}), 170.6 (C_{q}), 169.8 (C_{q}), 169.6 (2×C_{q}), 167.0 (C_{q}), 151.3 (C_{q}), 138.9 (C_{q}), 137.5 (C_{q}), 130.9 (C_{q}), 130.1 (CH), 129.1 (2×CH), 128.1 (CH), 127.6 (CH), 127.5 (2×CH), 127.0 (CH), 78.9 (CH), 73.6 (CH), 72.6 (CH), 70.8 (CH), 68.3 (CH), 61.7 (CH₂), 47.7 (CH), 45.3 (CH), 30.2 (CH₂), 28.2 (CH₂), 23.5 (CH₃), 20.7 (CH₃), 20.6 (3×CH₃); HRMS (ESI) (M+H)⁺ m/z calculated for C₃₃H₃₉N₂O₁₁ 639.2548, found 639.2535.

### (1S,4S)—N,N-Dimethyl-1,2,3,4-tetrahydro-[1,2′-binaphthalen]-4-amine [VM-099]

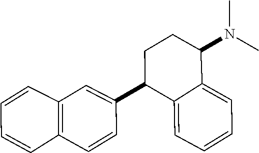 [SMILES: CN(C)[C@@H]1CC[C@H](c2ccc3ccccc3c2)c2ccccc21]

¹H NMR (400 MHz, CDCl₃) δ 7.78 (d, J=11.2 Hz, 3H), 7.66 (d, J=9.2 Hz, 1H), 7.61 (s, 1H), 7.50-7.40 (m, 2H), 7.27-7.16 (m, 2H), 7.09 (t, J=9.2 Hz, 1H), 6.79 (d, J=10 Hz, 1H), 4.30 (m, 2H), 2.47 (s, 6H), 2.37-2.29 (m, 2H), 2.34-2.17 (m, 2H), 1.98 (t, J=12.8 Hz, 2H); ¹³C NMR (100 MHz, CDCl₃) δ 145.4 [C], 142.8 [C], 135.0 [C], 133.8 [C], 131.1 [CH], 129.2 [CH], 129.0 [CH], 128.6 [2×CH], 128.4 [CH], 128.2 [CH], 127.8 [CH], 127.4 [CH], 127.1 [CH], 126.5 [CH], 64.5 [CH], 47.5 [CH], 40.6 [2×CH₃], 32.5 [CH₂], 20.8 [CH₂], a quaternary carbon is not visible; HRMS (ES+) calculated for C₂₂H₂₄N [M+H]⁺ 302.1909, found 302.1904.

2. Biological Study

2.1 Interaction with TCTP

The interaction of the synthesized molecules with TCTP was evaluated by the surface plasmon resonance (SPR) technique using the Biacore T200 apparatus.

A beam of polarized monochromatic light illuminates a glass interface between two media with different refractive indices, since the angle of incidence is greater than the limiting angle, all the light is reflected, a phenomenon known as total internal reflection. There is then no refraction but an electromagnetic component of the light, the evanescent wave propagates over a distance equivalent to its own wavelength, perpendicular to the interface. At the level of the gold leaf located at the interface between the two media, a resonance between the gold plasmons and the evanescent wave is observed, which results in a loss of light energy in the beam reflected at a precise angle called the resonance angle. This angle is sensitive to the refractive index of the medium wherein the evanescent wave propagates.

A continuous measurement of the variations in the angle of refraction is carried out using a micro-refractometer producing a sensorgram that allows the fixation of the molecules injected into the microfluidic cell to be monitored in real time. The resonance signal is expressed in resonance units (RU) and reports what is present at a given time in the microfluidic cell.

Thus, TCTP is bonded to a chip consisting of a layer of dextran on a gold foil, which in turn is placed on a glass plate. This layer is sandwiched between a microfluidic cell and a prism.

The synthesized compounds are passed through the microfluidic cell and their interaction is evaluated through the received SPR signal (Table 1).

### 2.2. Cytotoxic Activity

The cytotoxic activity of the prepared compounds was evaluated on the human cancer line HCT116 (colorectal carcinoma). The selected line was incubated at 37° C. in the presence of one of the prepared compounds added to the culture medium at various concentrations. The inhibitory concentration inducing 50% cell death (IC₅₀) was determined after 72 hours incubation for each compound (Table 1).

TABLE 1

| | | Kd SPR | IC₅₀ |
|---|---|---|---|
| Number | Structure | (μM) | (μM) |
| AC085 | 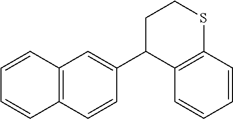 [SMILES: c1ccc2c(c1)SCCC2c1ccc2ccccc2c1] | 30 | 25 ± 1 |
| AC069 | 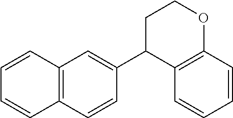 [SMILES: c1ccc2c(c1)OCCC2c1ccc2ccccc2c1] | 24 ± 16 | 1 μM: 0%. 10 μM: 33% (viability) |
| AC014 | 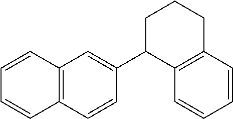 [SMILES: c1ccc2c(c1)CCCC2c1ccc2ccccc2c1] | 7 ± 3 | 8 ± 1 |
| AC082 | 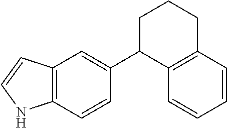 [SMILES: c1ccc2c(c1)CCCC2c1ccc2[nH]ccc2c1] | 123 ± 33 | 1 μM: 9 ± 1%. 10 μM: 33% (viability) |
| AC056 | 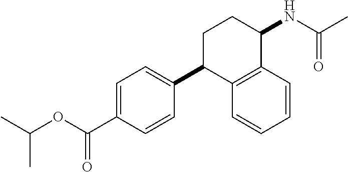 | 28.5 ± 10 | 17 ± 3 |
| AC088 | 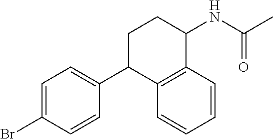 [SMILES: CC(=O)NC1CCC(c2ccc(Br)cc2)c2ccccc21] | 135 ± 37 | 10 μM: 33% (viability) |
| AC087 | 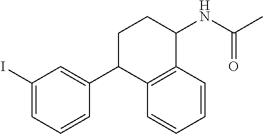 [SMILES: CC(=O)NC1CCC(c2cccc(I)c2)c2ccccc21] | 16 ± 11 | 10 μM: 33% (viability) |
| AC034 | 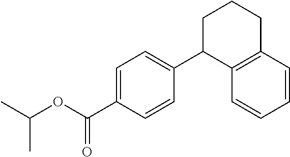 [SMILES: CC(C)OC(=O)c1ccc(C2CCCc3ccccc32)cc1] | 29 ± 7 | 25 ± 9 |
| AC041 | 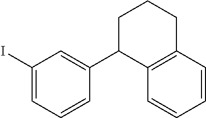 [SMILES: Ic1cccc(C2CCCc3ccccc32)c1] | 59 ± 30 | 9 ± 1.2 |
| AC096 | 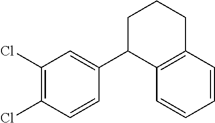 [SMILES: Clc1ccc(C2CCCc3ccccc32)cc1Cl] | 14 ± 7 | 1 μM: 29 ± 3%. |
| AC095 | 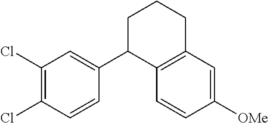 [SMILES: COc1ccc2c(c1)CCCC2c1ccc(Cl)c(Cl)c1] | 150 | 10 μM: 40% (viability) |
| AC101 | 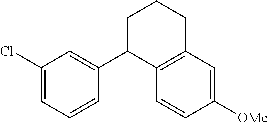 [SMILES: COc1ccc2c(c1)CCCC2c1cccc(Cl)c1] | 145 ± 34 | 7 ± 0.4 |
| AC249-cis | 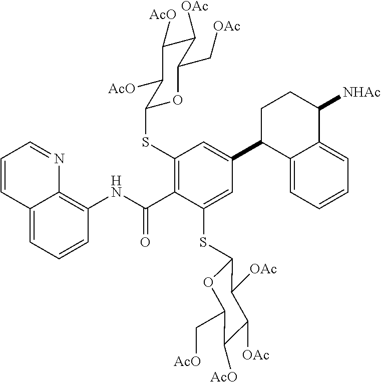 | 8 ± 2 μM | n.d. |
| AC268-cis | 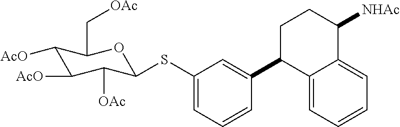 | 170 ± 31 μM | n.d. |
| VM060-cis | 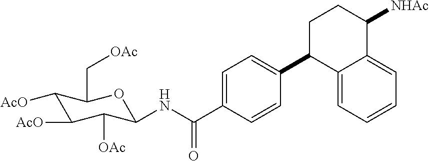 | 26 ± 8 μM | 10 μM: 15% (viability) |
| VM055-cis | 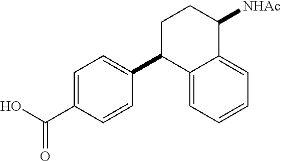 | 176 μM | n.d. |

n.d. not determined

Compounds AC014, AC096, AC087, AC069, AC085, AC056, AC034 and AC041 showed excellent affinity for TCTP and also have an antiproliferative activity profile identical to that of sertraline.

2.3 Overexpression of p53 Protein

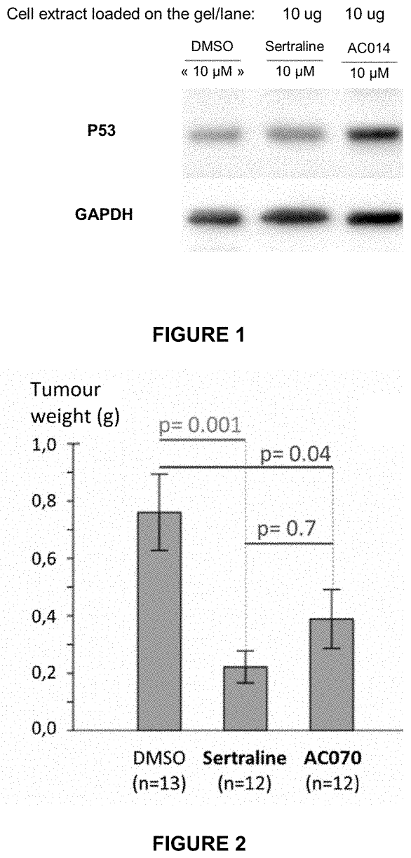

The compound AC014 is capable of inducing overexpression of p53 (FIG. 1). Western blot analyses show that compound AC014 induces at 10 μM, the expression of the p53 protein. This induction is more pronounced compared to that induced by sertraline.

The Western blot was carried out according to the protocol described in the following article: Amson R, Pece S, Lespagnol A, Vyas R, Mazzarol G, Tosoni D, Colaluca I, Viale G, Rodrigues-Ferreira S, Wynendaele J, Chaloin O, Hoebeke J, Marine J C, Di Fiore P P, Telerman A. Nat Med. 2011 Dec. 11; 18(1):91-9.

The following antibodies were used in the Western blot assay: anti-TCTP antibodies were generated against the whole human-derived TCTP. These polyclonal antibodies were purified by affinity column affinity coupled to TCTP (Agro-Bio). Anti-P53 1C12 mouse antibody (Cell Signaling Technology) was used at a dilution of 1/1000.

Secondary antibodies (antirabbit and antimouse) conjugated to HRP were used at a dilution of 1/5000 to visualize the signals by Western blots.

2.4 Inhibition of Cell Growth

The viability and proliferation of human HCT116 cells is measured using the “CellTiter Glo®” assay (Promega), which is a luminescent measure of the number of living cells. Sertraline and AC070 were tested at 10 concentrations and results are expressed as GI₅₀, TGI, LC₅₀ and IC₅₀ relative to control cells (DMSO).

The CellTiter-Glo® assay is a homogeneous method for determining the number of viable cells in culture based on a quantification of the ATP present, an indicator of metabolically active cells. The homogeneous test procedure involves the addition of a single reagent (the CellTiter-Glo® reagent) directly to the cultured cells in serum-enriched medium.

The results of the test are reported in the following Tables 2 and 3 (Table 2: sertraline and Table 3: AC070):

TABLE 2

| | | | | | Standard |
|---|---|---|---|---|---|
| GI₅₀ Median | 1 | 2 | 3 | Mean | deviation |
| 1.404E−07 | 1.251E−07 | 1.413E−07 | 1.590E−07 | 1.418E−07 | 1.698E−08 |
| | | | | | Standard |
| TGI₅₀ Median | 1 | 2 | 3 | Mean | deviation |
| 2.272E−07 | 2.031E−07 | 2.285E−07 | 2.533E−07 | 2.283E−07 | 2.509E−08 |
| | | | | | Standard |
| LC₅₀ Median | 1 | 2 | 3 | Mean | deviation |
| 3.555E−07 | 3.188E−07 | 3.559E−07 | 3.922E−07 | 3.556E−07 | 3.672E−08 |

TABLE 3

| | | | | | Standard |
|---|---|---|---|---|---|
| GI₅₀ Median | 1 | 2 | 3 | Mean | deviation |
| 8.886E−07 | 9.091E−07 | 8.810E−07 | 8.783E−07 | 8.895E−07 | 1.704E−08 |
| | | | | | Standard |
| TGI₅₀ Median | 1 | 2 | 3 | Mean | deviation |
| 1.442E−06 | 1.480E−06 | 1.439E−06 | 1.406E−06 | 1.442E−06 | 3.694E−08 |
| | | | | | Standard |
| LC₅₀ Median | 1 | 2 | 3 | Mean | deviation |
| 2.282E−06 | 2.344E−06 | 2.298E−06 | 2.196E−06 | 2.279E−06 | 7.569E−08 |

Sertraline has a cell growth inhibition constant GI₅₀=141 nM and a lethal concentration LC₅₀=355 nM while AC070 has a GI₅₀=889 nM and a LC₅₀=2.2 μM. AC070 is therefore slightly less active than sertraline on cell growth but is less cytotoxic than sertraline.

2.5. In Vivo Mouse Model Test on Each C57BL/6 Mouse

The compound AC070 and sertraline were tested in vivo in C57BL/6 mice. In a first step, induced tumor reversion-1 (ITR-1) tumor cells were collected from sarcomas developed by p53/− knock-out (ko) mice. Then, one million of these cells were injected (SC) into each C57BL/6 mouse to develop tumors. Once this step was completed, the compounds to be analyzed (sertraline and compound AC070) were injected into the C57BL/6 mice (30 mg/kg 1×/d by IP route). This study was conducted as a double-blind study. After 12 days, the mice were sacrificed and their tumors were weighed and analyzed. The results of these analyses are shown in diagrammatic form (FIG. 2). The first column of the diagram corresponds to an injection of dimethylsulfoxide (DMSO) without the presence of any active agent and serves as a reference for the study. The p-value or “significance” was calculated by software using the Tukey method, and is significant if it is less than 0.05, which corresponds to a margin of error of 5%.

1-13. (canceled)

14. A method for treating a proliferative or infectious disease, an allergy, an inflammation and/or an asthma comprising administering to a patient in need thereof an effective amount of a compound of the following formula (I): 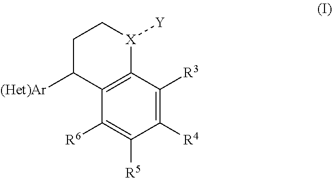
Wherein
X represents an oxygen atom, a sulfur atom, a nitrogen atom or a CH radical,
The bond X—Y and Y are absent if X represents an oxygen or sulfur atom, the bond X—Y and Y are present if X represents a nitrogen atom or a CH radical,
When present, Y represents
a group R if X represents a nitrogen atom,
wherein R represents a hydrogen atom, a C₁ to C₆ alkyl group, an aryl group, a heteroaryl group, a (C₂-C₆)alkenyl group, a (C₂-C₆)alkynyl group or an acyl group,
a hydrogen atom or a group —NR¹R² if X represents a CH radical,
wherein R¹ and R² represent, independently of one another, a hydrogen atom, a C₁ to C₆ alkyl group, an aryl group, a heteroaryl group, a (C₂-C₆)alkenyl group, a (C₂-C₆)alkynyl group or an acyl group, or R¹ and R², together with the nitrogen atom carrying them, form a 5- or 6-membered heterocyclic ring,
(Het)Ar is an aromatic ring selected from the group consisting of aryl and heteroaryl groups,
said aromatic ring may be substituted by one or more groups selected from a halogen atom, a —COOR⁷ group, a —CONR⁸R⁹ group, a C₁ to C₆ alkyl group, a —SR¹⁰ group, a CF₃ group, a formyl group, an OR¹¹ group, a (C₂-C₆)alkenyl group,
with R⁷ representing a hydrogen atom, a C₁ to C₆ alkyl group, an aryl group, a heteroaryl group or a sugar residue,
with R⁸ and R⁹ representing, independently of one another, a hydrogen atom, a C₁ to C₆ alkyl group, an aryl group, a heteroaryl group, a (C₂-C₆)alkenyl group, a (C₂-C₆)alkynyl group, a sugar residue, an amino acid residue, a peptide residue or R⁸ and R⁹, together with the nitrogen atom carrying them, form a 5- or 6-membered heterocyclic ring,
with R¹⁰ representing a hydrogen atom, a C₁ to C₆ alkyl group, an aryl group, a heteroaryl group, a sugar residue, a peptide residue comprising at least one cysteine or —SR¹⁰ represents a cysteine residue,
with R¹¹ representing a hydrogen atom, a C₁ to C₆ alkyl group, an aryl group or a benzyl group,
R³, R⁴, R⁵, R⁶ represent, independently of one another, a hydrogen atom, a halogen atom, an —NR¹²R¹³ group, a —SR¹⁴ group, an —OR¹⁴ group or a —CF₃ group,
where R¹² and R¹³ represent independently of one another a hydrogen atom, a C₁ to C₆ alkyl group, an aryl group, a heteroaryl group, a (C₂-C₆)alkenyl group, a (C₂-C₆)alkynyl group or an acyl group, or R¹² and R¹³, together with the nitrogen atom carrying them, form a 5- or 6-membered heterocyclic ring,
with R¹⁴ representing a hydrogen atom, a C₁-C₆ alkyl group, an aryl group, a heteroaryl group, a sugar residue, an amino acid residue or a peptide residue,
When Y represents a group —NR¹R², the groups —NR¹R² and (Het)Ar are in the cis-conformation,
the alcohol functions of the sugar residue and the amine functions of the amino acid residue, of the cysteine residue or of the peptide residue being in their free or protected form,
or a pharmaceutically acceptable salt thereof,
with the exception of the compound 4-(3,4-dichloro-phenyl)-1,2,3,4-tetrahydronaphthalene-1-ylamine or a pharmaceutically acceptable salt thereof.

15. The method according to claim 14, wherein the compound of formula (I) is a compound of the following formula (II): 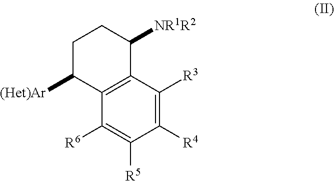
wherein R¹, R², R³, R⁴, R⁵, R⁶ and Het(Ar) are as defined in claim 14.

16. The method according to claim 15, wherein R¹ represents an acyl.

17. The method according to claim 14, wherein the compound of formula (I) is a compound of the following formula (III): 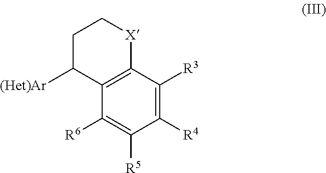
Wherein
X′ represents CH₂, O, S or N—R,
R, R³, R⁴, R⁵, R⁶ and Het(Ar) are as defined in claim 14.

18. The method according to claim 14, wherein (Het)Ar is an aromatic ring selected from the group consisting of phenyl or naphthyl groups,
wherein said aromatic ring is optionally substituted with a —COOR⁷ group, a —CONR⁸R⁹ group, a —SR¹⁰ group, a CF₃ group, or at most one halogen atom,
R⁷, R⁸, R⁹ and R¹⁰ being as defined in claim 14.

19. The method according to claim 14, wherein the compound of formula (I) or the pharmaceutically acceptable salt thereof is selected from: 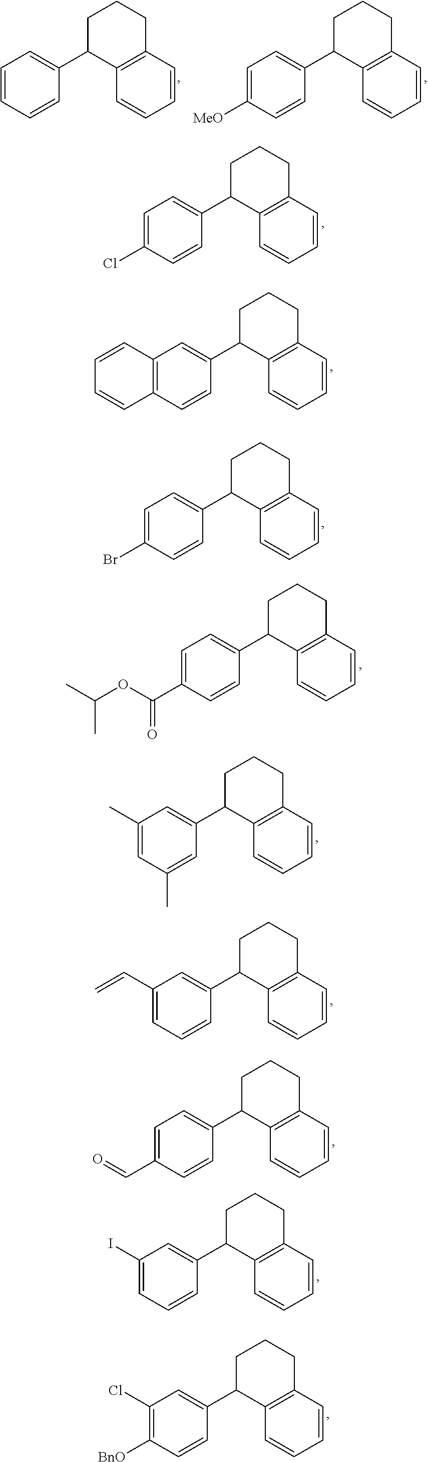 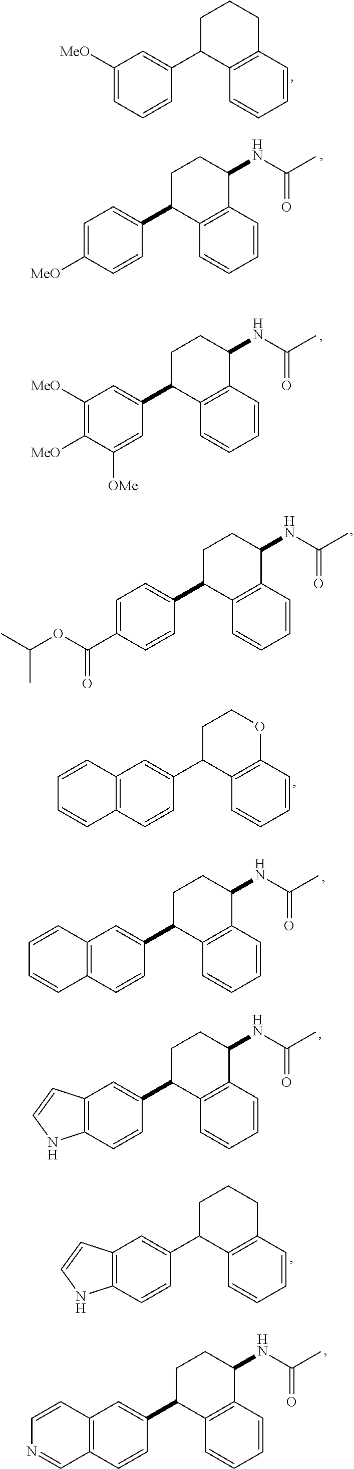 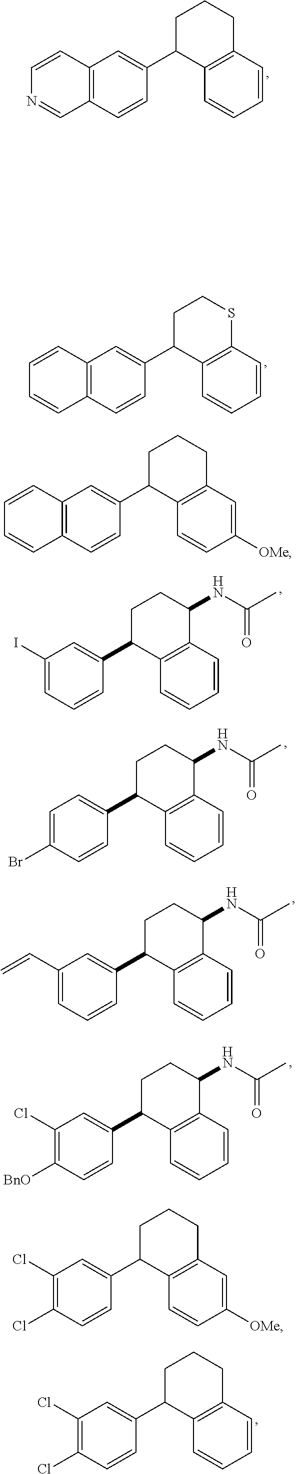 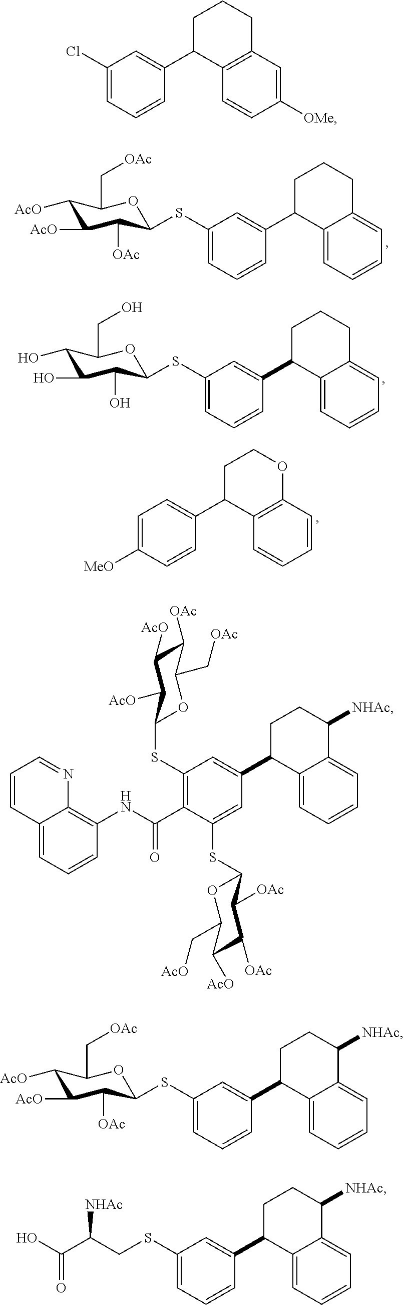 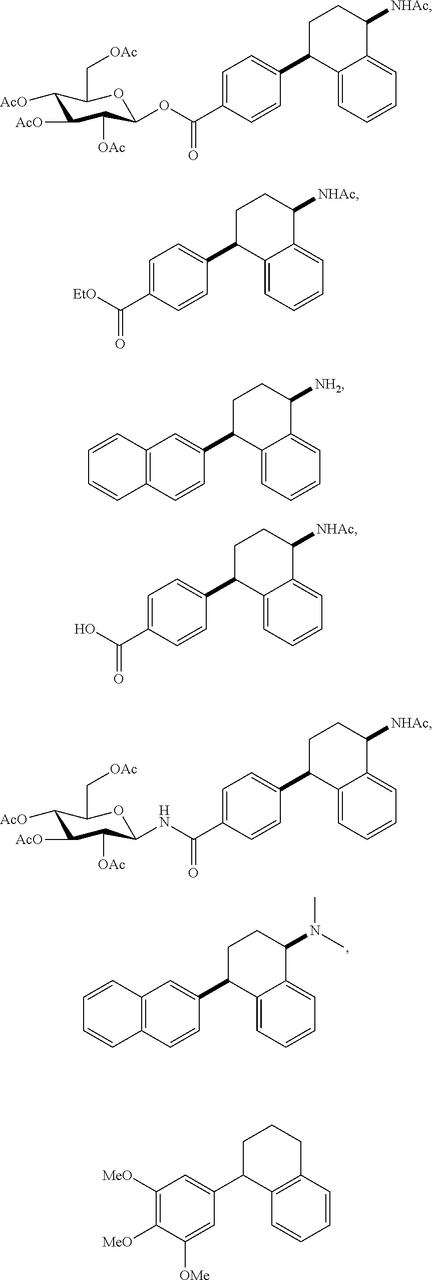
and their pharmaceutically acceptable salts.

20. The method according to claim 14, wherein the proliferative disease is a cancer.

21. The method according to claim 20, wherein the treatment of cancer is performed by tumor reversion.

22. The method according to claim 14, wherein the proliferative disease is an acute myeloid leukemia, a breast cancer or a brain cancer.

23. The method according to claim 14, wherein the compound of formula (I) or the pharmaceutically acceptable salt thereof is administered in association with another active agent.

24. The method according to claim 23, wherein the other active agent is an anticancer agent.

25. The method according to claim 24, wherein the anticancer agent is cytarabine.

26. The method according to claim 14, wherein the infectious disease is a parasitic infectious disease.

27. The method according to claim 26, wherein the infectious disease is malaria.

28. A method for treating a proliferative or infectious disease, an allergy, an inflammation and/or an asthma comprising administering to a patient in need thereof an effective amount of a pharmaceutical composition comprising a compound of formula (I) as defined in claim 14 or a pharmaceutically acceptable salt thereof and a pharmaceutically acceptable excipient.

29. A compound selected from: 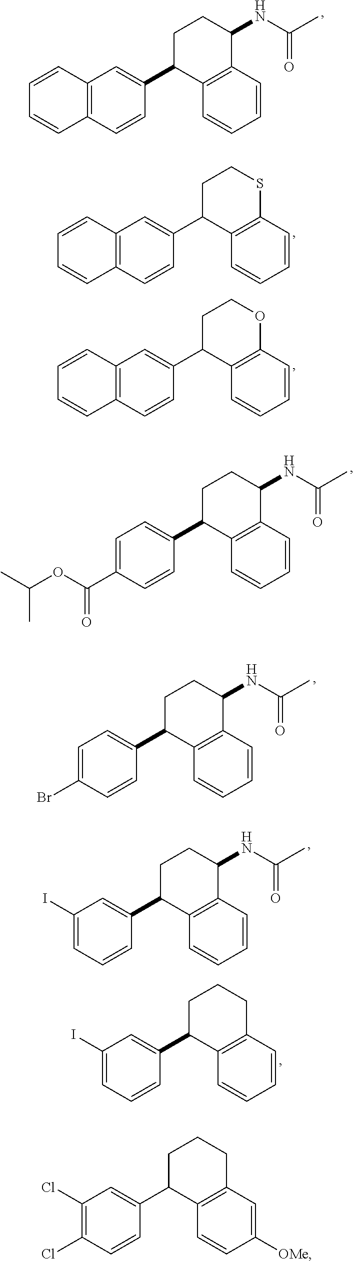 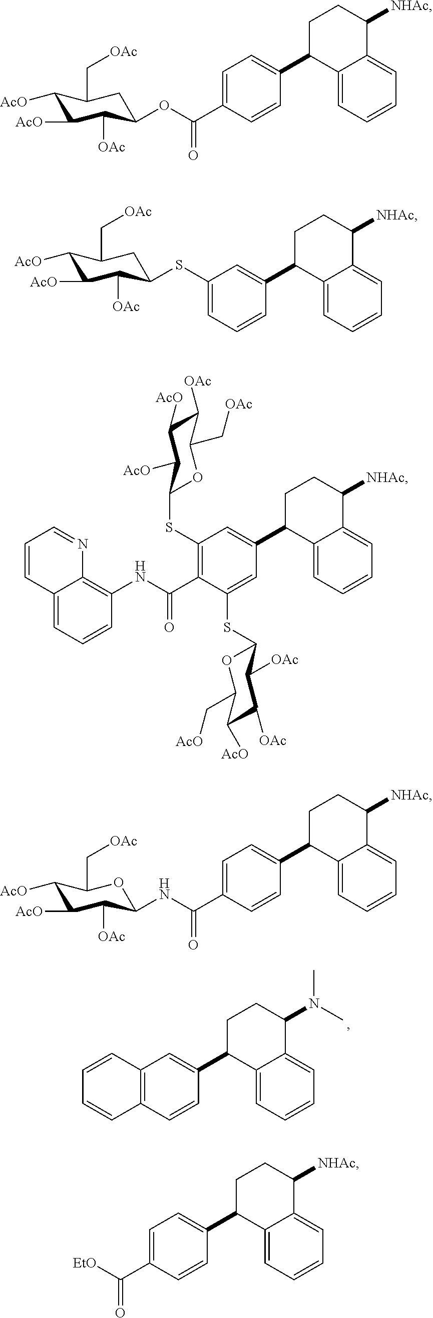 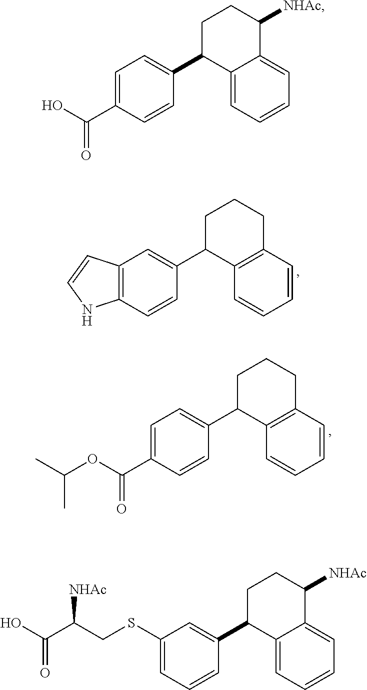
and their pharmaceutically acceptable salts.

30. A method for inhibiting translationally controlled tumor protein TCTP comprising administering to a patient in need thereof an effective amount of a compound of the following formula (I): 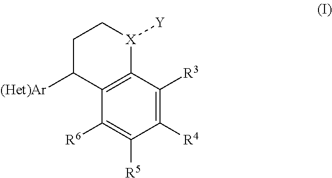
Wherein
X represents an oxygen atom, a sulfur atom, a nitrogen atom or a CH radical,
The bond X—Y and Y are absent if X represents an oxygen or sulfur atom, the bond X—Y and Y are present if X represents a nitrogen atom or a CH radical,
When present, Y represents
a group R if X represents a nitrogen atom,
wherein R represents a hydrogen atom, a C₁ to C₆ alkyl group, an aryl group, a heteroaryl group, a (C₂-C₆)alkenyl group, a (C₂-C₆)alkynyl group or an acyl group,
a hydrogen atom or a group —NR¹R² if X represents a CH radical,
wherein R¹ and R² represent, independently of one another, a hydrogen atom, a C₁ to C₆ alkyl group, an aryl group, a heteroaryl group, a (C₂-C₆)alkenyl group, a (C₂-C₆)alkynyl group or an acyl group, or R¹ and R², together with the nitrogen atom carrying them, form a 5- or 6-membered heterocyclic ring,
(Het)Ar is an aromatic ring selected from the group consisting of aryl and heteroaryl groups,
said aromatic ring may be substituted by one or more groups selected from a halogen atom, a —COOR⁷ group, a —CONR⁸R⁹ group, a C₁ to C₆ alkyl group, a —SR¹⁰ group, a CF₃ group, a formyl group, an OR¹¹ group, a (C₂-C₆)alkenyl group,
with R⁷ representing a hydrogen atom, a C₁ to C₆ alkyl group, an aryl group, a heteroaryl group or a sugar residue,
with R⁸ and R⁹ representing, independently of one another, a hydrogen atom, a C₁ to C₆ alkyl group, an aryl group, a heteroaryl group, a (C₂-C₆)alkenyl group, a (C₂-C₆)alkynyl group, a sugar residue, an amino acid residue, a peptide residue or R⁸ and R⁹, together with the nitrogen atom carrying them, form a 5- or 6-membered heterocyclic ring,
with R¹⁰ representing a hydrogen atom, a C₁ to C₆ alkyl group, an aryl group, a heteroaryl group, a sugar residue, a peptide residue comprising at least one cysteine or —SR¹⁰ represents a cysteine residue,
with R¹¹ representing a hydrogen atom, a C₁ to C₆ alkyl group, an aryl group or a benzyl group,
R³, R⁴, R⁵, R⁶ represent, independently of one another, a hydrogen atom, a halogen atom, an —NR¹²R¹³ group, a —SR¹⁴ group, an —OR¹⁴ group or a —CF₃ group,
where R¹² and R¹³ represent independently of one another a hydrogen atom, a C₁ to C₆ alkyl group, an aryl group, a heteroaryl group, a (C₂-C₆)alkenyl group, a (C₂-C₆)alkynyl group or an acyl group, or R¹² and R¹³, together with the nitrogen atom carrying them, form a 5- or 6-membered heterocyclic ring,
with R¹⁴ representing a hydrogen atom, a C₁-C₆ alkyl group, an aryl group, a heteroaryl group, a sugar residue, an amino acid residue or a peptide residue,
When Y represents a group —NR¹R², the groups —NR¹R² and (Het)Ar are in the cis-conformation,
the alcohol functions of the sugar residue and the amine functions of the amino acid residue, of the cysteine residue or of the peptide residue being in their free or protected form,
or a pharmaceutically acceptable salt thereof,
with the exception of the compound 4-(3,4-dichloro-phenyl)-1,2,3,4-tetrahydronaphthalene-1-ylamine or a pharmaceutically acceptable salt thereof.

31. The method according to claim 30, wherein the compound of formula (I) is a compound of the following formula (II): 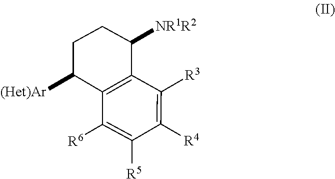
wherein R¹, R², R³, R⁴, R⁵, R⁶ and Het(Ar) are as defined in claim 30.

32. The method according to claim 31, wherein R¹ represents an acyl.

33. The method according to claim 30, wherein (Het)Ar is an aromatic ring selected from the group consisting of phenyl or naphthyl groups,
wherein said aromatic ring is optionally substituted with a —COOR⁷ group, a —CONR⁸R⁹ group, a —SR¹⁰ group, a CF₃ group, or at most one halogen atom,
R⁷, R⁸, R⁹ and R¹⁰ being as defined in claim 30.
